(19) United States
(12) Patent Application Publication
(10) Pub. No.: US 2021/0228635 A1
(43) Pub. Date: Jul. 29, 2021

(54) THERAPEUTIC DOSAGE REGIMENS COMPRISING ADHERENT STROMAL CELLS

(71) Applicant: PLURISTEM LTD., Haifa (IL)

(72) Inventors: Zami ABERMAN, Tel-Mond (IL); Yaacob YANAY, Shimshit (IL); Rachel OFIR, Adi (IL); Marc TRITEL, Bet Shemesh (IL)

(73) Assignee: PLURISTEM LTD., Haifa (IL)

(21) Appl. No.: 15/734,605

(22) PCT Filed: Jun. 10, 2019
(86) PCT No.: PCT/IB2019/054828

§ 371 Date: Dec. 3, 2020

Related U.S. Application Data
(60) Provisional application No. 62/689,882, filed on Jun. 26, 2018.
(60) Provisional application No. 62/714,786, filed on Aug. 6, 2018.
(60) Provisional application No. 62/757,221, filed on Nov. 8, 2018.
(60) Provisional application No. 62/683,300, filed on Jun. 11, 2018.

(51) Int. Cl. A61K 35/28; C12N 5/073; A61K 35/50

(52) U.S. Cl. CPC A61K 35/28; C12N 2513/00; A61K 35/50; C12N 5/0605

(57) ABSTRACT

Disclosed herein are pharmaceutical compositions and therapeutic dosage regimens comprising or utilizing adherent stromal cells. The adherent stromal cells may be derived e.g. from placental tissue, from adipose tissue, or from bone marrow. The pharmaceutical compositions may be indicated for treating various disorders, e.g. ischemic disorders, hematopoietic disorders, and neurodegenerative disorders, inflammatory disorders, and neoplasms. The pharmaceutical compositions may further include pharmacologically acceptable excipients.

### FIELD

Disclosed herein are therapeutic dosage regimens comprising adherent stromal cells.

### BACKGROUND

The HLA system or complex is a gene complex encoding the major histocompatibility complex (MHC) proteins in humans. These cell-surface proteins are involved in regulation of the immune system in humans. The HLA gene complex resides on a 3-Mbp stretch within chromosome 6p21. HLA genes are polymorphic. HLAs encoding MHC class I proteins (“class I HLA's”) present peptides from inside the cell, while class II HLA's present external peptides.

There are 3 major MHC class I genes, HLA-A, HLA-B, and HLA-C; and 3 minor class I genes, HLA-E, HLA-F and HLA-G. The protein β2-microglobulin binds with major and minor gene subunits to produce a heterodimer.

There are 3 major (DP, DQ and DR) and 2 minor (DM and DO) MHC class II proteins encoded by the HLA. The class II MHC proteins combine to form heterodimeric (αβ) protein receptors that are typically expressed on the surface of antigen-presenting cells.

Adherent stromal cells (ASC) are known for use in cell therapy. Methods for improved cell therapy are urgently needed in the art.

### SUMMARY

In some embodiments, there is provided a therapeutic method, said method comprising the steps of: (a) administering a first pharmaceutical composition, comprising allogeneic adherent stromal cells (ASC) from a first donor; and subsequently (b) administering a second pharmaceutical composition comprising allogeneic ASC from a second donor, wherein the second donor differs from the first donor in at least one allele group of human leukocyte antigen (HLA)-A or human leukocyte antigen (HLA)-B.

In certain embodiments, the ASC are derived from a placenta. In other embodiments, the ASC are derived from adipose tissue, or BM. In other embodiments, the ASC are derived from a different source tissue.

In still other embodiments, there is a provided a method of treating an ischemic disorder, a hematopoietic disorder, a neurodegenerative disorder, an inflammatory disorder, or a neoplasm, comprising administering the described pharmaceutical compositions, e.g. according to the described regimens.

In another embodiment, there is provided use of the described ASC populations in the preparation of a medicament. In various embodiments, the ASC are derived from a placenta or from adipose tissue, or BM.

In other embodiments, there is provided a therapeutic method, said method comprising administering a pharmaceutical composition, comprising ASC, to a subject, wherein the ASC has been determined to possess an HLA type, and said subject has been tested for immunity against said HLA type. In certain embodiments, the subject has been determined to lack significant immunity against said HLA type.

In still other embodiments, there is provided a therapeutic method, said method comprising the steps of: (a) testing a subject for immunity against a panel of HLA types; (b) selecting an ASC population from a group of populations (in some embodiments, from different donors), wherein the populations exhibit common characteristics but differ in their HLA types, and the subject lacks significant immunity against the HLA type of the selected population; and (c) administering a pharmaceutical composition, comprising the selected ASC population, to the subject. Thus, in some embodiments, the selected population is chosen based (at least in part) on an expected lack of significant immune reactivity of the subject for the population.

When comparing the characteristics of 2 or more populations, those skilled in the art will appreciate that, preferably, the characteristics are compared by a side-by-side assay. Alternatively, the different populations can be compared in separate experiments, using side-by-side assays with the same reference standard, to which the results are normalized.

In yet another embodiment, there is provided a therapeutic method, said method comprising the steps of: (a) administering a first pharmaceutical composition, comprising allogeneic ASC from a first donor, to a subject; (b) testing the subject for immunity against a panel of HLA types; subsequently (c) selecting a second ASC population from a group of populations, wherein the populations exhibit characteristics common to the ASC from the first donor, but differ in their HLA types; and (d) administering a second pharmaceutical composition, comprising the second ASC population, to the subject. Thus, in some embodiments, the subject lacks significant immunity against the HLA type of the second ASC population, and/or the second population is chosen based (at least in part) on an expected lack of significant immune reactivity of the subject for the population.

In certain embodiments, the ASC are derived from a placenta. In other embodiments, the ASC are derived from adipose tissue, or BM. In other embodiments, the ASC are derived from a different source tissue.

In still other embodiments, there is a provided a method of treating an ischemic disorder, a hematopoietic disorder, a neurodegenerative disorder, an inflammatory disorder, an orthopedic condition, or a neoplasm, comprising administering pharmaceutical compositions as per the described procedures or regimens.

In certain embodiments, the ASC described herein have been cultured on a 2-dimensional (2D) substrate, a 3-dimensional (3D) substrate, or a combination thereof. Non-limiting examples of 2D and 3D culture conditions are provided in the Detailed Description and in the Examples.

Reference herein to “growth” of a population of cells is intended to be synonymous with expansion of a cell population. In certain embodiments, ASC (which may be, in certain embodiments, placental ASC), are expanded without substantial differentiation. In various embodiments, the described expansion is on a 2D substrate, on a 3D substrate, or a 2D substrate, followed by a 3D substrate.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting. Unless otherwise indicated, Uniprot and GenBank Nos. mentioned herein were accessed on Jun. 2, 2019.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIG. 2 depicts the study visit flow chart for the study described in Example 5.

FIG. 3 is a chart showing characteristics of placental ASC expanded in 2D, then 3D culturing, then removed from the carriers. CV % indicates the coefficient of variance, obtained by dividing the standard deviation by the average, and multiplying ×100.

FIGS. 4A-C are charts showing stimulation of endothelial cell proliferation and VEGF secretion by ASC (A-B), and IL-10 secretion by monocytes co-incubated with ASC (C) for 3 representative batches of placental ASC that were expanded in 2D, then 3D culturing, then removed from the carriers. For A and C, the vertical axis is percentage activity of the reference batch, while for B, the vertical axis shows picograms per milliliter (pg./ml) of VEGF.

FIGS. 5A-C are charts showing percent viability (A), percent recovery (B) and percent of cell adhesion (C) of the 3 representative batches examined in the previous figure.

FIG. 6 contains charts depicting secretion of IL-6, HGF, Gro-alpha (GROa), IL-8, SDF-1 alpha, IGFBP-1, Osteoprotegerin, and Angiogenin (A); Angiopoietin-1, IGFBP-3, MIF, FLRG, Osteopontin, and Galectin-1 (B); and Serpin E1, MMP-1, TIMP1, secreted Beta2 microglobulin, and MMP-2 (C) by Luminex® assay. Vertical axis: protein levels in the CM in pg./ml.

FIG. 7 contains charts depicting secretion of HGF, Angiogenin, and Angiopoietin-1 (A); Decorin (not tested for 27) and Osteopontin (not tested for 09), and (B); Galectin-1 and MMP-2 (C) by ELISA; and M-CSF, PDGF-BB, and FGF-7 (D) by RayBiotech array. Vertical axis: protein levels in in the CM in pg./ml.

FIG. 8A-D are charts depicting percent proliferation of PBMC in the presence of ASC. For each ICS/source placenta, 4 bioreactor runs were tested in parallel and tested with both PBMC donors. Values were normalized to PHA-stimulated control without ASC. Means and standard errors were obtained from the 8 samples. Vertical axis shows normalized numbers of cells that underwent at least 1 (A), at least 2 (B), at least 3 (C), or at least 4 (D) divisions.

FIG. 9 contains charts depicting the mean (A) and adjusted mean (B) log MWD change of subjects in the FAS2Rx receiving placebo (dashed line) or 2 injections of 300 million ASC from 2 different placentas (dotted line) or the same placenta (solid line). Bars depict the standard error.

FIG. 10 is a graph showing reduction from baseline CRP levels in subjects who received ASC from 2 different placentas (white circles) or 2 doses from the same placenta (diamonds), or placebo (PBO-PBO) (black circles), in the mFAS 300-300 population. Vertical axis: adjusted means +/−SE of change in blood CRP (nmol/L). Horizontal axis: study week.

FIG. 11 is a graph showing MWD in patients exhibiting (blank circles) or not exhibiting (filled circles) anti-HLA antibodies at visit 5. Horizontal axis: Time (weeks). Vertical axis: In (natural log) MWD change.

FIG. 12A is a perspective view of a carrier (or “3D body”), according to an exemplary embodiment. B and C are perspective and cross-sectional views of embodiments of a carrier.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise allogeneic adherent stromal cells (ASC). In some embodiments, the ASC are derived from placenta; while in other embodiments, the ASC are derived from adipose tissue. In still other embodiments, the ASC are derived from bone marrow (BM). In still other embodiments, the ASC are derived from another tissue. In certain embodiments, both the subject and the ASC donor are human.

In some embodiments, there is provided a therapeutic method, said method comprising the steps of: (a) administering a first pharmaceutical composition, comprising allogeneic ASC from a first donor; and subsequently (b) administering a second pharmaceutical composition comprising allogeneic ASC from a second donor, wherein the second donor differs from the first donor in at least one allele group of HLA-A or HLA-B; and wherein the administrations are separated in time from each other by at least 7 days. Since each donor has 2 allele groups for each of HLA-A and HLA-B, the donors thus differ in at least one of the 4 allele groups.

Also provided herein are allogeneic ASCs for use in a therapeutic method, said method comprising the steps of: (a) administering a first pharmaceutical composition, comprising allogeneic ASC from a first donor; and subsequently (b) administering a second pharmaceutical composition comprising allogeneic ASC from a second donor, wherein the second donor differs from the first donor in at least one allele group of HLA-A or HLA-B; and wherein the administrations are separated in time from each other by at least 7 days.

Allogeneic, as used herein (except where indicated otherwise), refers to a biological material (e.g. ASC) not derived from, and not syngeneic with, the subject being treated. Typically, allogeneic ASC are neither syngeneic nor haploidentical with the subject.

In certain embodiments, the described allogeneic ASC from the first donor and the second donor (also referred to herein as “first ASC population” and “second ASC population”, respectively) are derived from the same tissue, which may be, in some embodiments, placenta. In other embodiments, the tissue is adipose, or is bone marrow. In still other embodiments, the tissue is another source of ASC.

Alternatively or in addition, the first ASC population and second ASC population exhibit one or more common characteristics. In some embodiments, the common characteristics relate to the cells' therapeutic potential. Certain embodiments of such common characteristics are described herein. In other embodiments, at least one common characteristic is selected from population doubling time (PDL; this parameter may be derived from population doubling level) and glucose consumption rate (GCR). In other embodiments, the common characteristics include both PDG and GCR. In certain embodiments, the PDL and/or GCR are measured in bioreactor culture in 3D fibrous carriers, e.g. as described herein in Example 4, following cell expansion as described in Example 1. In certain embodiments, the 2 populations are within 2 fold of each other for GCR on day 5 of bioreactor culture. In other embodiments, the GCR is measured on day 3, day 4, or day 6. Alternatively or in addition, the 2 populations are within 1.5 fold, within 3 fold, or within 5 fold of each other for the specified parameter(s).

In some embodiments, there is provided a method of delivering a therapeutic moiety to a subject in need thereof, comprising the steps of: a) administering to a subject a first pharmaceutical composition, comprising allogeneic ASC from a first donor; and b) administering to the subject, at least 7 days after step a), a second pharmaceutical composition comprising allogeneic ASC from a second donor, wherein the allogeneic ASC from the 2 donors comprise, or in more specific embodiments secrete, the therapeutic moiety, and wherein the second donor differs from the first donor in at least one allele group of HLA-A or HLA-B, thereby delivering a therapeutic moiety. In certain embodiments, the therapeutic moiety is a secreted therapeutic moiety.

In other embodiments, there is provided a therapeutic method, said method comprising administering to a subject a pharmaceutical composition, comprising ASC, wherein the ASC have been selected from a group of populations that exhibit common characteristics but differ in their HLA types, and said subject has been tested for immunity against an HLA type of the selected ASC population. In certain embodiments, the subject has been determined to lack significant immunity against the HLA type of the selected ASC population. HLA type, in preferred embodiments, may refer to an HLA-A type. In other embodiments, HLA type refers to both HLA-A and HLA-B. In still other embodiments, HLA type refers to HLA-A, HLA-B, and HLA-C. In yet other embodiments, HLA type refers to HLA-A, HLA-B, and HLA-DR.

In other embodiments, there is provided a method of delivering a therapeutic moiety to a subject in need thereof, said method comprising administering to a subject a pharmaceutical composition, comprising an ASC population, wherein the ASC population has been selected from a group of populations that exhibit common characteristics but differ in their HLA types, wherein the cells in the ASC population comprise, or in more specific embodiments secrete, the therapeutic moiety, and said subject has been tested for immunity against said HLA type. In certain embodiments, the subject has been determined to lack significant immunity against said HLA type. HLA type, in preferred embodiments, may refer to an HLA-A type. In other embodiments, HLA type refers to both HLA-A and HLA-B. In still other embodiments, HLA type refers to HLA-A, HLA-B, and HLA-C. In yet other embodiments, HLA type refers to HLA-A, HLA-B, and HLA-DR.

In other embodiments, there is provided a therapeutic method, said method comprising administering to a subject a pharmaceutical composition, comprising ASC, wherein the ASC have been selected from a group of populations that exhibit common characteristics but differ in their HLA types. In this method, prior to administration of the pharmaceutical composition, the subject has been administered allospecific desensitization against said HLA type. Methods for allospecific desensitization are known in the art, a non-limiting example of which is reduction of antibody titer levels of the recipient. Non-limiting examples of such methods are described, for example in Alelign T et al. HLA type, in preferred embodiments, may refer to an HLA-A type. In other embodiments, HLA type refers to both HLA-A and HLA-B. In still other embodiments, HLA type refers to HLA-A, HLA-B, and HLA-C. In yet other embodiments, HLA type refers to HLA-A, HLA-B, and HLA-DR.

In still other embodiments, there is provided a therapeutic method, said method comprising the steps of: (a) testing a subject for immunity against a panel of HLA types; (b) selecting an ASC population from a group of populations from different donors, wherein the populations exhibit common characteristics but differ in at least one HLA allele, and the subject lacks significant immunity against the HLA type of the selected population; and (c) administering a pharmaceutical composition, comprising the selected ASC population, to the subject. Thus, in some embodiments, the selected population is chosen based (at least in part) on an expected lack of significant immune reactivity of the subject for the population. The described HLA allele may refer, in preferred embodiments, to an HLA-A allele. In other embodiments, the populations differ in both HLA-A alleles; or, in other embodiments, at least one allele of both HLA-A and HLA-B; or, in other embodiments, at least one allele each of HLA-A, HLA-B, and HLA-C; or, in still other embodiments, at least one allele each of HLA-A, HLA-B, and HLA-DR.

In still other embodiments, there is provided a method of delivering a therapeutic moiety to a subject in need thereof, said method comprising the steps of: (a) testing a subject for immunity against a panel of HLA types; (b) selecting an ASC population from a group of populations from different donors, wherein the populations exhibit common characteristics but differ in their HLA types, the cells of the ASC population comprise, or in other embodiments secrete, the therapeutic moiety, and the subject lacks significant immunity against the HLA type of the selected population; and (c) administering a pharmaceutical composition, comprising the selected ASC population, to the subject. Thus, in some embodiments, the selected population is chosen based (at least in part) on an expected lack of significant immune reactivity of the subject for the population.

In yet another embodiment, there is provided a therapeutic method, said method comprising the steps of: (a) administering a first pharmaceutical composition, comprising allogeneic ASC from a first donor, to a subject; (b) testing the subject for immunity against a panel of HLA types; (c) selecting a second ASC population from a group of populations, wherein the populations in the panel exhibit characteristics common to the ASC from the first donor, but differ in their HLA types, and the subject lacks significant immunity against the HLA type of the second ASC population; and (d) administering a second pharmaceutical composition, comprising the second ASC population, to the subject. Thus, in some embodiments, the second population is chosen based (at least in part) on an expected lack of significant immune reactivity of the subject for the population. In certain embodiments, the subject is tested for allospecific immunity after the first pharmaceutical composition is administered; while in other embodiments, the subject may be tested for allospecific immunity before the first pharmaceutical composition is administered. HLA type, in preferred embodiments, may refer to an HLA-A type. In yet other embodiments, the first and second ASC populations differ in their HLA types. In other embodiments, HLA type refers to both HLA-A and HLA-B. In still other embodiments, HLA type refers to HLA-A, HLA-B, and HLA-C. In yet other embodiments, HLA type refers to HLA-A, HLA-B, and HLA-DR.

In yet another embodiment, there is provided a method of delivering a therapeutic moiety to a subject in need thereof, said method comprising the steps of: (a) administering a first pharmaceutical composition, comprising allogeneic ASC from a first donor, to a subject; (b) testing the subject for immunity against a panel of HLA types; (c) selecting a second ASC population from a group of populations, wherein the populations each secrete therapeutic levels of the therapeutic moiety but differ in their HLA types, and the subject lacks significant immunity against the HLA type of the second ASC population; and (d) administering a second pharmaceutical composition, comprising the second ASC population, to the subject. Thus, in some embodiments, the second population is chosen based (at least in part) on an expected lack of significant immune reactivity of the subject for the population. In certain embodiments, the subject is tested for allospecific immunity after the first pharmaceutical composition is administered; while in other embodiments, the subject may be tested for allospecific immunity before the first pharmaceutical composition is administered.

Significant immunity to an HLA type (allospecific immunity), as used herein, refers to a level of immunity that is expected to result in acute rejection of a tissue having the specified HLA type (Alelign T et al). Those skilled in the art will appreciate that specificity of a subject's HLA antibodies can be determined using Luminex-based assays, which may utilize, for example, fluorescent microbeads conjugated to single recombinant HLA class I and class II molecules. Such kits are commercially available, and include, for example, the One Lambda kit (ThermoFisher) and the LIFECODES LSA Single Antigen kit (Immucor).

In other embodiments, HLA antibodies present in the serum of the subject are assayed for complement-fixing ability, e.g. binding of C1q to the antibodies. Lack of complement-fixing ability above threshold levels in standard assays (Valenzuela and Reed; Chin et al) indicates immune tolerance.

In yet other embodiments, HLAMatchmaker (Silva et al.) is used to evaluate compatibility of the subject with the described HLA populations.

In certain embodiments, the described therapeutic moiety is a secreted protein. In other embodiments, the therapeutic moiety is exosomes. In still other embodiments, the therapeutic moiety is VEGF (vascular endothelial growth factor A; Uniprot Accession No. P15692). In certain embodiments, the levels of VEGF secreted by the 2 populations are within 2-fold of one another. In other embodiments, the secretion levels of the 2 populations are within 1.5 fold, 3 fold, or 5 fold of one another. Those skilled in the art will appreciate, in light of this disclosure, that secretion levels of VEGF (or other cytokines) can be measured by methods known in the art. One possible method is seeding 1×10⁶ ASC for 20 hours in 2 mL DMEM medium; replacing the medium with EBM-2 medium, and incubating the cells under hypoxic conditions (1% O₂) for an additional 24 hours; and collecting the conditioned media (CM). VEGF levels in the CM are then measured by ELISA. This is referred to herein as the “standard ELISA protocol” or “standard protocol”.

Alternatively or in addition, the first and second ASC populations both secrete between 300-700 picograms per milliliter (pg./ml) (as exemplified herein) of VEGF per 10⁶ cells seeded, using the standard protocol. In other embodiments, the 2 populations both secrete at least 200, at least 300, at least 400, between 250-1,000, between 250-800, between 250-700, between 300-1,000, between 300-800, between 300-600, between 400-1000, between 400-800, between 400-700, or between 400-600 pg./ml of VEGF per 10⁶ cells seeded, using the standard protocol. Those skilled in the art will appreciate that, since the standard protocol is performed in 2 ml medium, the number of picograms per 10⁶ cells seeded is twice the number of pg./ml, per 10⁶ cells seeded. Thus, the value of 300-700 pg./ml per 10⁶ cells seeded translates to 600-1400 pg. per 10⁶ cells. Similarly, the other aforementioned values are equivalent to least 400, at least 600, at least 800, between 500-2,000, between 500-1600, between 500-1400, between 600-2,000, between 600-1600, between 600-1200, between 800-2000, between 800-1600, between 800-1400, and between 800-1200 pg. (respectively) of VEGF per 10⁶ cells seeded.

In yet other embodiments, the therapeutic moiety is PDGF. In certain embodiments, the levels of PDGF secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of PDGF can be measured by methods known in the art, e.g. the standard ELISA protocol.

Alternatively or in addition, the first and second ASC populations both secrete between 400-1200 pg. of PDGF-BB per 0.5×10⁶ cells seeded, using the standard protocol. In other embodiments, the 2 populations both secrete at least 200, at least 300, at least 400, between 400-2,000, between 400-1500, between 500-2,000, between 500-1500, or between 500-1200 pg. of PDGF-BB per 0.5×10⁶ cells seeded, using the standard protocol.

In still other embodiments, the therapeutic moiety is selected from Angiogenin (Uniprot accession no. P03950), Angiopoietin 1 (Uniprot accession no. Q15389), MCP-1, IL-8, Serpin E1, and GCP2/CXCL6 (Uniprot accession no. P80162).

In yet other embodiments, the therapeutic moiety is selected from IL-17, MCP-1, IL-2, CCL4/MIP-1b (Accession No. P13236), IL-4, TGF-b, TNF-alpha, IL-19, IL-20, IL-23, ADAM10-processed FasL form (sFAS; a cleavage product of TNFL6 [Accession No. P48023]), Cox-2, CXCL12, CSF1, MMP-2, MMP-9, IL-32 (Accession No. P24001).

In yet other embodiments, the therapeutic moiety is IL-6 (UniProt No. P05231). In certain embodiments, the levels of IL-6 secreted by the 2 populations are within 2-fold of one another. In other embodiments, the secretion levels of the 2 populations are within 1.5 fold, 3 fold, or 5 fold of one another. Those skilled in the art will appreciate, in light of this disclosure, that secretion levels of IL-6 can be measured by methods known in the art, e.g. the standard ELISA protocol.

Alternatively or in addition, the first and second ASC populations both secrete between 20-160 pg. (=10-80 pg./ml, as exemplified herein) of IL-6 per 10⁶ cells seeded, using the standard protocol. In other embodiments, the 2 populations both secrete at least 12, at least 16, at least 20, at least 24, at least 30, at least 40, between 12-200, between 16-200, between 20-200, between 24-200, between 30-200, between 12-400, between 16-400, between 20-400, between 24-400, or between 30-400 pg. of IL-6 per 10⁶ cells seeded, using the aforementioned standard protocol.

In still other embodiments, the first and second ASC populations both secrete between 100,000-400,000 pg. of IL-6 (=50,000-200,000 pg./ml; as exemplified in WO 2018/198012, which is incorporated herein by reference) per 2×10⁵ cells seeded, using the induced protocol (described herein). In yet other embodiments, both populations secrete at least 60,000, at least 80,000, at least 100,000, at least 120,000, at least 140,000, at least 160,000, at least 180,000, at least 200,000; or within one of the ranges 100,000-300,000, 120,000-300,000, 140,000-300,000, 160,000-300,000, 100,000-260,000, 120,000-260,000, 140,000-260,000, 160,000-260,000, 100,000-220,000, 120,000-220,000, 140,000-220,000, or 160,000-220,000 pg. of IL-6 per 2×10⁵ cells seeded, using the induced protocol.

In other embodiments, the therapeutic moiety is any other factor mentioned herein.

Additionally or alternatively, each ASC population secretes or expresses (as appropriate in each case) IL-6, IL-8, eukaryotic translation elongation factor 2 (EEEF2), reticulocalbin 3, EF-hand calcium binding domain (RCN₂), and/or calponin 1 basic smooth muscle (CNN1), when tested using the aforementioned standard protocol. In more specific embodiments, the 2 populations (or in other embodiments 3 populations, or more than 3 populations) of cells secrete at least one, in other embodiments at least 2, in other embodiments at least 3, in other embodiments at least 4, in other embodiments all 5 of the aforementioned proteins, at levels within 2-fold of one another. In other embodiments, the secretion levels of the 2 populations are within 1.5 fold, 3 fold, or 5 fold of one another

Reference herein to “secrete”/“secreting”/“secretion” relates to a detectable secretion of the indicated factor, above background levels in standard assays. For example, 0.5×10⁶ fetal or maternal ASC can be suspended in 4 ml medium (DMEM+10% fetal bovine serum (FBS)+2 mM L-Glutamine), added to each well of a 6 well-plate, and cultured for 24 hrs in a humidified incubator (5% CO₂, at 37° C.). After 24 h, DMEM is removed, and cells are cultured for an additional 24 hrs in 1 ml RPMI 1640 medium +2 mM L-Glutamine+0.5% HSA. The CM is collected from the plate, and cell debris is removed by centrifugation.

Also provided herein are allogeneic ASCs for use in a method of delivering a therapeutic moiety to a subject in need thereof, said method comprising the steps of: (a) administering a first pharmaceutical composition, comprising a first allogeneic ASC population; and subsequently (b) administering a second pharmaceutical composition comprising a second allogeneic ASC population from a second donor, who differs from the first donor in at least 1 allele group of HLA-A or HLA-B; wherein the administrations are separated in time from each other by at least 7 days.

Also provided herein is a method of stimulating angiogenesis in a subject in need thereof, said method comprising the steps of: (a) administering a first pharmaceutical composition, comprising a first ASC population; and subsequently (b) administering a second pharmaceutical composition comprising a second ASC population, wherein the second donor differs from the first donor in at least one allele group of HLA-A or HLA-B; wherein the administrations are separated in time from each other by at least 7 days.

Also provided, in still other embodiments, is a method of treating an ischemic disorder, comprising the steps of: a) administering to a subject a first pharmaceutical composition, comprising a first ASC population; and b) administering to the subject, at least 7 days after step a), a second pharmaceutical composition comprising a second ASC population, wherein the second donor differs from the first donor in at least one allele group of HLA-A or HLA-B, thereby treating an ischemic disorder. In certain embodiments, the ischemic disorder is a peripheral artery disease (PAD). Alternatively or in addition, the ASC secrete a factor(s) that stimulates angiogenesis, which may be, in some embodiments, any factor mentioned herein. In certain embodiments, the ischemic disorder is critical limb ischemia (CLI). In other embodiments, the ischemic disorder is intermittent claudication (IC). In still other embodiments, the ischemic disorder is selected from ischemia of the central nervous system (CNS) (e.g. ischemic stroke), ischemic heart disease and ischemic renal disease. Other relevant embodiments are described in WO 2009/037690, which is incorporated herein by reference.

Also provided herein are ASCs for use in a method for treating an ischemic disorder (which is, in some embodiments, PAD), said method comprising the steps of (a) administering a first pharmaceutical composition, comprising a first ASC population; and subsequently (b) administering a second pharmaceutical composition comprising a second ASC population, wherein the second donor differs from the first donor in at least one allele group of HLA-A or HLA-B; wherein the administrations are separated in time from each other by at least 7 days. Also provided is use of the described first and second pharmaceutical compositions in the manufacture of a medicament for treating an ischemic disorder. In other embodiments, there is provided an article of manufacture, comprising a packaging material and the described first and second pharmaceutical compositions identified for treating an ischemic disorder, the pharmaceutical compositions being contained within the packaging material. In some embodiments, the indication is specified in a leaflet that is included within the article of manufacture.

In certain embodiments, the described first ASC population and second ASC population are derived from the same tissue, which may be, in some embodiments, placenta. In other embodiments, the tissue is adipose, or is bone marrow. In still other embodiments, the tissue is another source of ASC.

Alternatively or in addition, the first ASC population and second ASC population both stimulate endothelial cell proliferation (ECP). In more specific embodiments, the ex-vivo ECP-stimulating activities of the 2 populations are within 2-fold of one another. In other embodiments, the activities of the 2 populations are within 1.5 fold, 3 fold, or 5 fold of one another. Those skilled in the art will appreciate that ECP activity can be assayed ex vivo by seeding 1×10⁶ ASC in 2 mL DMEM medium, in wells of a 6-well plate for 20 hours, then replacing the medium with EBM-2 medium (available from Sigma-Aldrich) and incubating the cells under hypoxic conditions (1% O₂) for an additional 24 hours. Afterwards, the conditioned media (ASC-CM) is collected. 750 Human Umbilical Vein Endothelial Cells (HUVECs) cells are seeded per well of 96-well plate were seeded and incubated for 24 hours in EBM-2 medium and then incubated with the ASC-CM, for 4 days under normoxic conditions (21% O₂) at 37° C., and proliferation is assayed.

In other embodiments, the described first ASC population and second ASC populations both secrete the same therapeutic moiety, which is, in some embodiments, a secreted protein. In still other embodiments, the therapeutic moiety is VEGF. In certain embodiments, the levels of VEGF secreted by the 2 populations are within 2-fold of one another. In other embodiments, the secretion levels of the 2 populations are within 1.5 fold, 3 fold, or 5 fold of one another. In other embodiments, the therapeutic moiety is VEGF. In certain embodiments, the levels of VEGF secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of VEGF can be measured by methods known in the art, e.g. the described standard ELISA protocol.

Alternatively or in addition, the first and second ASC populations both secrete between 600-2000 pg. (=300-1000 pg./ml, as exemplified herein) of VEGF per 10⁶ cells seeded, using the standard protocol described herein. In other embodiments, the 2 populations both secrete at least 400, at least 600, at least 800, between 600-1600, between 600-1400, between 600-1200, between 800-2000, between 800-1600, between 800-1400, or between 800-1200 pg. of VEGF per 10⁶ cells seeded, using the standard protocol.

In still other embodiments, the first and second ASC populations both secrete between 2000-5000 pg. (=1,000-2,500 pg./ml; as exemplified in WO 2018/198012, which is incorporated herein by reference) of VEGF per 2×10⁵ cells seeded, using the induced protocol described herein. In yet other embodiments, both populations secrete at least 1000, at least 1600, at least 2,000, at least 3,000, between 2,000-6,000, between 2400-6000, between 2400-5000, between 3000-6000, between 3000-5000, between 3000-4600, between 3000-4400, between 3000-4200, or between 3000-4000 pg. of VEGF per 2×10⁵ cells seeded, using the induced protocol.

In yet other embodiments, the therapeutic moiety is Angiogenin. In certain embodiments, the levels of Angiogenin secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of Angiogenin can be measured by methods known in the art, e.g. the described standard ELISA protocol.

Alternatively or in addition, the first and second ASC populations both secrete between 400-800 pg. (=200-400 pg./ml, as exemplified herein) of Angiogenin per 10⁶ cells seeded, using the standard protocol. In other embodiments, the 2 populations both secrete at least 100, at least 200, at least 300, at least 400, at least 500; or within one of the ranges 200-1000, 300-1000, 400-1000, 200-800, 300-800, 500-800, or 500-700 pg. of Angiogenin per 10⁶ cells seeded, using the standard protocol.

In yet other embodiments, the therapeutic moiety is Serpin E1. In certain embodiments, the levels of Serpin E1 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of Serpin E1 can be measured by methods known in the art, e.g. the described standard ELISA protocol. Alternatively or in addition, the 2 ASC populations both secrete between 30,000-60,000 pg. (=15,000-30,000 pg./ml, as exemplified herein) of Serpin E1 per 10⁶ cells seeded, using the standard protocol. In other embodiments, the 2 populations both secrete at least 10,000, at least 12,000, at least 16,000, at least 20,000, at least 24,000, at least 30,000, or within one of the ranges 20,000-60,000, 20,000-50,000, 20,000-40,000, 24,000-60,000, 24,000-50,000, 24,000-40,000, 30,000-60,000, 30,000-40,000, 30,000-50,000, 30,000-48,000, 30,000-46,000, 32,000-48,000, 32,000-46,000, 30,000-100,000, or 30,000-80,000 pg. of Serpin E1 per 10⁶ cells seeded, using the standard protocol.

In yet other embodiments, the therapeutic moiety is MMP-1. In certain embodiments, the levels of MMP-1 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of MMP-1 can be measured by methods known in the art, e.g. the described standard ELISA protocol.

Alternatively or in addition, the first and second ASC populations both secrete between 8000-400,000 pg. (=4000-200,000 pg./ml, as exemplified herein) of MMP-1 per 10⁶ cells seeded, using the standard protocol. In other embodiments, the 2 populations both secrete between 8000-300,000 pg. of MMP-1 per 10⁶ cells seeded. In still other embodiments, the 2 populations both secrete between 8000-200,000 pg. of MMP-1 per 10⁶ cells seeded. In other embodiments, the 2 populations both secrete between 8000-160,000 pg. of MMP-1 per 10⁶ cells seeded. In other embodiments, the 2 populations both secrete at least 6000, at least 8000, at least 10,000, at least 12,000; or within one of the ranges 10,000-200,000, 10,000-160,000, 10,000-140,000, 12,000-200,000, 12,000-160,000, 12,000-140,000, or 12,000-120,000 pg. of MMP-1 per 10⁶ cells seeded, using the standard protocol.

In still other embodiments, the first and second ASC populations both secrete Flt-3 ligand (Fms-related tyrosine kinase 3 ligand; Uniprot Accession No. P49772), stem cell factor (SCF; Accession No. P21583), IL-6, or combinations thereof, each of which represents a separate embodiment. In certain embodiments, the 2 or more populations of ASC secrete levels of Flt-3 ligand, SCF, and IL-6—or in other embodiments 2 or more of these cytokines, or in other embodiments all 3 cytokines—that are within 2-fold of one another, using the standard protocol. In other embodiments, the secreted levels are within 1.5 fold, 3 fold, or 5 fold of one another.

In other embodiments, the first and second ASC populations both secrete 2 or more, in other embodiments 3 or more, in other embodiments 4 or more, in other embodiments 5 or more, in other embodiments 6 or more, in other embodiments 7 or more, or in other embodiments all of the factors VEGF, Angiogenin, PDGF, Angiopoietin 1, MCP-1, IL-8, Serpin E1, and GCP2/CXCL6. In other embodiments, the ASC secrete VEGF, Angiogenin, Angiopoietin 1, MCP-1, IL-8, and Serpin E1, which were found to be secreted by maternal cells. In still other embodiments, the ASC secrete VEGF, Angiogenin, Angiopoietin 1, MCP-1, IL-8, Serpin E1, and GCP2/CXCL6, which were found to be secreted by fetal cells. In certain embodiments, the cytokine levels secreted by the 2 or more ASC populations are within 2-fold of one another, using the standard protocol. In other embodiments, the secreted levels are within 1.5 fold, 3 fold, or 5 fold of one another.

In still other embodiments, the first and second ASC populations both secrete 2 or more, in other embodiments 3 or more, in other embodiments 4 or more, in other embodiments 5 or more, in other embodiments 6 or more, or in other embodiments 7 or more factors selected from MCP-1 (CCL2), Osteoprotegerin, MIF (Macrophage migration inhibitory factor; Uniprot Accession No. P14174), GDF-15, SDF-1 alpha, GROa (Growth-regulated alpha protein/CXCL1; No. P09341), Beta2-Microglobulin (beta2M; this protein, although it forms complexes with the heavy chain of MHC class I, can also be secreted [Nomura T et al]), IL-6, IL-8 (No. P10145), ENA78/CXCL5, eotaxin/CCL11 (Acc. No. P51671), and MCP-3 (CCL7). In certain embodiments, the ASC secrete MCP-1, Osteoprotegerin, MIF, GDF-15, SDF-1 alpha, GROa, beta2M, IL-6, IL-8, and MCP-3, which were found to be secreted by maternal cells. In other embodiments, the ASC secrete MCP-1, Osteoprotegerin, MIF, GDF-15, SDF-1 alpha, beta2M, IL-6, IL-8, ENA78, eotaxin, and MCP-3, which were found to be secreted by fetal cells. In certain embodiments, the cytokine levels secreted by the 2 or more ASC populations are within 2-fold of one another, using the standard protocol. In other embodiments, the secreted levels are within 1.5 fold, 3 fold, or 5 fold of one another.

In some embodiments, there is provided a method of treating a neurodegenerative disorder, comprising the steps of: a) administering to a subject a first pharmaceutical composition, comprising a first ASC population; and b) administering to the subject, at least 7 days after step a), a second pharmaceutical composition comprising a second ASC population, wherein the second donor differs from the first donor in at least one allele group of HLA-A or HLA-B, thereby treating a neurodegenerative disorder. In some embodiments, the neurodegenerative disorder is selected from Alzheimer's disease, Parkinson's disease, ALS, and Huntington's disease.

In certain embodiments, the described first ASC population and second ASC population are derived from the same tissue, which may be, in some embodiments, placenta. In other embodiments, the tissue is adipose, or is bone marrow. In still other embodiments, the tissue is another source of ASC.

Alternatively or in addition, the ASC secrete one or more neurotrophic and/or neuroprotective factors. In certain embodiments, the described first ASC population and second ASC populations both secrete the same therapeutic moiety, which is, in some embodiments, a secreted protein. In certain embodiments, the therapeutic moiety is BDNF (brain derived neurotrophic factor; Uniprot Accession No. P23560).

In yet other embodiments, the therapeutic moiety is BDNF. In certain embodiments, the levels of BDNF secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of BDNF in CM from ASC can be measured by methods known in the art. In certain embodiments, CM is produced by culturing 2×10⁵ ASC per well in 6-well plates, in 2 ml DMEM +10% FBS medium per well. After 1 day, the medium is aspirated, and the cells are washed in PBS and cultured for 72 hours in DMEM without serum supplemented with 1 mM (millimolar) dbcAMP, 20 ng/ml (nanograms per milliliter) bFGF, 50 mcg/ml (microgram/milliliter) heparin, and N-2 animal-free cell culture supplement (ThermoFisher Scientific, Cat. #1752048 to 1× concentration (as provided in International Appl. Publ. No. WO 2018/198012, to Niva Shraga-Heled and Rachel Ofir), which is incorporated herein by reference; “induced protocol”).

Alternatively or in addition, the first and second ASC populations both secrete between 300-600 pg. (=150-300 pg./ml) of BDNF per 2×10⁵ cells seeded, using the aforementioned induced protocol. In other embodiments, the 2 ASC populations secrete at least 50, at least 60, at least 70, at least 80, at least 100, at least 120, at least 150, at least 200, between 100-300, between 120-300, or between 150-250 pg./ml of BDNF per 2×10⁵ cells seeded, using the induced protocol. Those skilled in the art will appreciate that, since the induced protocol is performed in 2 ml medium, the number of picograms per 10⁶ cells seeded is twice the number of pg./ml, per 10⁶ cells seeded. Thus, the value of 150-300 pg./ml per 10⁶ cells seeded translates to 300-600 pg./ml per 10⁶ cells. The other aforementioned values are equivalent to at least 100, at least 120, at least 140, at least 160, at least 200, at least 240, at least 300, at least 400, between 200-600, between 240-600, or between 300-500 pg. of BDNF per 2×10⁵ cells seeded.

In yet other embodiments, the therapeutic moiety is HGF (hepatocyte growth factor; Uniprot Accession No. P14210). In certain embodiments, the levels of HGF secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of HGF can be measured by methods known in the art, e.g. the induced protocol.

Alternatively or in addition, the first and second ASC populations both secrete between 80,000-160,000 pg. (=40,000-80,000 pg./ml, as provided in WO 2018/198012, which is incorporated herein by reference) of HGF per 2×10⁵ cells seeded, using the induced protocol. In yet other embodiments, both populations secrete at least 60,000, at least 80,000, at least 100,000, at least 120,000, between 60,000-160,000, between 60,000-140,000, between 80,000-140,000, between 100,000-160,000, between 100,000-140,000, between 110,000-140,000, between 120,000-150,000, or between 120,000-140,000 pg. of HGF per 2×10⁵ cells seeded, using the induced protocol.

In still other embodiments, the first and second ASC populations both secrete between 100-160 pg. (=50-80 pg./ml, as exemplified herein) of HGF per 10⁶ cells seeded, using the aforementioned standard ELISA protocol. In yet other embodiments, both populations secrete at least 50, at least 60, at least 80, at least 100, between 50-400, between 50-300, between 50-200, between 50-160, between 60-400, between 60-300, between 60-200, between 60-160, between 80-400, between 80-300, between 80-200, between 80-160, between 100-400, between 100-300, or between 100-200 pg. of HGF per 10⁶ cells seeded, using the standard ELISA protocol.

In other embodiments, the therapeutic moiety is GDNF (glial cell line derived neurotrophic factor; Uniprot Accession No. P39905). In certain embodiments, the levels of GDNF secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of GDNF can be measured by methods known in the art, e.g. the described induced protocol.

Alternatively or in addition, the first and second ASC populations both secrete between 140-300 pg. (=70-150 pg./ml; as exemplified in WO 2018/198012, which is incorporated herein by reference) of GDNF per 2×10⁵ cells seeded, using the induced protocol. In other embodiments, the 2 populations both secrete at least 100, at least 120, at least 140, at least 160, at least 180, at least 200, between 140-300, between 160-300, between 180-3000, between 140-280, between 160-280, between 180-280, between 140-260, between 160-260, or between 180-260 pg. of GDNF per 2×10⁵ cells seeded, using the induced protocol.

In yet other embodiments, the therapeutic moiety is IGFBP-1 (Insulin-like growth factor-binding protein 1; Uniprot Accession No. P08833). In certain embodiments, the levels of IGFBP-1 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of IGFBP-1 can be measured by methods known in the art, e.g. the described standard ELISA protocol.

Alternatively or in addition, the first and second ASC populations both secrete between 220-500 pg. (=110-250 pg./ml, as exemplified herein) of IGFBP-1 per 10⁶ cells seeded, using the standard protocol. In other embodiments, the 2 populations secrete at least 160, at least 200, at least 240, at least 300, between 160-600, between 160-500, between 200-600, between 200-500, between 240-600, between 240-500, between 300-600, between 300-500, between 300-450, between 350-500, between 350-400, or between 300-400 pg. of IGFBP-1 per 10⁶ cells seeded.

In yet other embodiments, the therapeutic moiety is IGFBP-3. In certain embodiments, the levels of IGFBP-3 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of IGFBP-3 can be measured by methods known in the art, e.g. the described standard ELISA protocol.

Alternatively or in addition, the first and second ASC populations both secrete between 2,000-14,000 pg. (=1000-7000 pg./ml; as exemplified herein) of IGFBP-3 per 10⁶ cells seeded, using the standard protocol. In other embodiments, the 2 populations secrete at least 1000, at least 1600, at least 2000, at least 3000, between 1000-12,000, between 100-10,000, between 1000-9000, between 2000-12,000, between 2000-10,000, between 2000-9000, between 3000-12,000, between 3000-10,000, or between 3000-9000 pg. of IGFBP-3 per 10⁶ cells seeded.

In other embodiments, the therapeutic moiety is selected from bFGF (basic fibroblast growth factor; Unitprot Accession no. P09038), NGF (nerve growth factor; Accession No. P01138), VEGF, LIF (Leukemia inhibitory factor; Accession No. P15018), MIF, MCP-1, PDGF (a non-limiting example of which is PDGF-AA), Angiogenin, IGFBP-3, and G-CSF. In yet other embodiments, the factor is selected from M-CSF, SDF-1, IFN-g, MMP-1, BMP-4 (Bone morphogenetic protein 4; Accession No. P12644), HB-EGF (Proheparin-binding EGF-like growth factor; Accession No. Q99075), GM-CSF, and ENA78. Other moieties are described in WO2018/198012, which is incorporated herein by reference. In more specific embodiments, the amounts of the therapeutic moiety secreted by the 2 populations are within 2-fold of one another. In other embodiments, the amounts of the moiety are within 1.5 fold, 3 fold, or 5 fold of one another.

Also provided herein are ASCs for use in a method for treating a neurodegenerative disorder, said method comprising the steps of (a) administering a first pharmaceutical composition, comprising a first ASC population; and subsequently (b) administering a second pharmaceutical composition comprising a second ASC population, wherein the second donor differs from the first donor in at least one allele group of HLA-A or HLA-B; and wherein the administrations are separated in time from each other by at least 7 days. Also provided is use of the described first and second pharmaceutical compositions in the manufacture of a medicament for treating a neurodegenerative disorder. In other embodiments, there is provided an article of manufacture, comprising a packaging material and the described first and second pharmaceutical compositions identified for treating a neurodegenerative disorder, the pharmaceutical compositions being contained within the packaging material. In some embodiments, the indication is specified in a leaflet that is included within the article of manufacture.

In some embodiments, there is provided a method of treating an inflammatory disorder, comprising: a) the step of administering to a subject a first pharmaceutical composition, comprising a first ASC population; and b) the step of administering to the subject, at least 7 days after step a), a second pharmaceutical composition comprising a second ASC population, wherein the second donor differs from the first donor in at least one allele group of HLA-A or HLA-B, thereby treating an inflammatory disorder.

In certain embodiments, the inflammatory disorder is selected from systemic lupus erythematosus (SLE), rheumatoid arthritis, systemic sclerosis, Sjorgen's syndrome, multiple sclerosis (MS), Myasthenia Gravis (MG), Guillain-Barre Syndrome, Hashimoto's Thyroiditis (HT), Graves's Disease, Insulin dependent Diabetes Melitus (IDDM), and Inflammatory Bowel Disease. Alternatively or in addition, the ASC secrete immunoregulatory and/or anti-inflammatory factor(s), which may be, in some embodiments, any factor mentioned herein. Other relevant embodiments are described in WO/2007/108003, which is incorporated herein by reference.

In certain embodiments, the described first ASC population and second ASC population are derived from the same tissue, which may be, in some embodiments, placenta. In other embodiments, the tissue is adipose, or is bone marrow. In still other embodiments, the tissue is another source of ASC.

Alternatively or in addition, the first ASC population and second ASC population both exhibit immunosuppressive capability. In more specific embodiments, the ex-vivo immunosuppressive activities of the 2 populations in an MLR assay are within 2-fold of one another. In other embodiments, the activities of the 2 populations are within 1.5 fold, 3 fold, or 5 fold of one another. Those skilled in the art will appreciate that immunosuppressive capability can be assayed ex vivo by mixed lymphocyte reaction (MLR). For example, human irradiated cord blood (iCB) cells are incubated with allogeneic human peripheral blood-derived monocytes (PBMC), in the presence or absence of a cell population to be tested. PBMC cell replication, which correlates with the intensity of the immune response, can be measured by ³H-thymidine uptake. Reduction of the PBMC cell replication when co-incubated with test cells indicates an immunosuppressive capability.

Methods of determining the immunosuppressive capability of a cell population are known to those skilled in the art, and exemplary methods are described in Example 3 of PCT Publication No. WO 2009/144720, which is incorporated herein by reference. For example, a mixed lymphocyte reaction (MLR) may be performed. In an exemplary, non-limiting MLR assay, irradiated cord blood (iCB) cells, for example human cells or cells from another species, are incubated with peripheral blood-derived monocytes (PBMC; for example human PBMC or PBMC from another species), in the presence or absence of a cell population to be tested. PBMC cell replication, which correlates with the intensity of the immune response, can be measured by a variety of methods known in the art, for example by ³H-thymidine uptake. Reduction of the PBMC cell replication when co-incubated with test cells indicates an immunosuppressive capability. Alternatively or in addition, secretion of pro-inflammatory and anti-inflammatory cytokines by blood cell populations (such as monocytes or PBMC) can be measured when stimulated (for example by incubation with non-matched cells, or with a non-specific stimulant such as PHA), in the presence or absence of the ASC. In certain embodiments, for example in the case of human ASC, as provided in WO 2009/144720, which is incorporated herein by reference, when 200,000 PBMC are co-incubated for 48 hours with 4,000 allogeneic ASC, followed by a 5-hour stimulation with 1.5 mcg/ml of LPS, the amount of IL-10 secretion by the PBMC is at least 120%, at least 130%, at least 150%, at least 170%, at least 200%, or at least 300% of the amount observed with LPS stimulation in the absence of ASC. In certain embodiments, the inhibition of T cell proliferation, expressed as the percent decrease in T cell proliferation relative to a control lacking ASC, varies less than 20% (or in other embodiments, less than 10%). By way of example, 2 populations that inhibit T cell proliferation by 30% and 48% vary between them in 48−30=18%.

In other embodiments, the first ASC population and second ASC population both increase secretion of IL-10 (Uniprot Accession No. P22301) by allogeneic monocytes over basal secretion, when the ASC are cocultured with the monocytes. Those skilled in the art will appreciate in light of the present disclosure that IL-10 secretion can be assayed by seeding 3000 ASC/well in X-VIVO™ 15 medium +10% FBS in 48-well plates and, 1 day later, co-incubating the ASC with 2×10⁴ U937 cells and incubating for 17 hours. PHA is then added, cells are incubated for another 5 hours, and IL-10 in the supernatant is measured by ELISA. In certain embodiments, the amount of IL-10 secretion by the monocytes is at least 120%, at least 130%, at least 150%, at least 170%, at least 200%, or at least 300% of the amount observed with LPS stimulation in the absence of ASC. Alternatively or in addition, the IL-10-stimulating activities of the 2 populations, expressed as a fold secretion relative to monocytes without ASC, are within 2-fold of one another. For example, if population A and population B elicit IL-10 secretion that is 1.8 and 3.6 of monocytes without ASC, then the IL-10-stimulating activities of the 2 populations are said to differ by 2-fold, and thus be within 2-fold of one another.

In other embodiments, the described first and second ASC populations both secrete the same therapeutic moiety, which is, in some embodiments, an immunoregulatory factor(s). In some embodiments, the therapeutic moiety is Leukemia Inhibitory Factor (LIF). In certain embodiments, the levels of LIF secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of LIF can be measured by methods known in the art, e.g. the described induced protocol.

Alternatively or in addition, the first and second ASC populations both secrete between 200-500 pg. (=100-250 pg./ml; as exemplified in WO 2018/198012, which is incorporated herein by reference) of LIF per 2×10⁵ cells seeded, using the induced protocol. In other embodiments, the 2 populations both secrete at least 100, at least 120, at least 140, at least 160, at least 180, at least 200, between 100-400, between 120-400, between 140-400, between 160-400, between 180-400, between 200-400, between 220-400, between 200-360, or between 220-360 pg. of LIF per 2×10⁵ cells seeded, using the induced protocol.

In still other embodiments, the therapeutic moiety is GROa (Growth-regulated alpha protein). In certain embodiments, the levels of GROa secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of GROa can be measured by methods known in the art, e.g. the described standard ELISA protocol.

Alternatively or in addition, the first and second ASC populations both secrete between 40-200 pg. (=100 pg./ml, as exemplified herein) of GROa per 10⁶ cells seeded, using the standard protocol. In other embodiments, the 2 ASC populations both secrete at least 60, at least 80, at least 100, at least 120, between 80-160, between 100-160, between 120-160, between 80-140, between 100-140, or between 120-140 pg. of GROa per 10⁶ cells seeded, using the standard protocol.

In still other embodiments, the therapeutic moiety is IL-8. In certain embodiments, the levels of IL-8 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of IL-8 can be measured by methods known in the art, e.g. the described standard ELISA protocol.

Alternatively or in addition, the first and second ASC populations both secrete between 100-700 pg. (=50-350 pg./ml, as exemplified herein) of IL-8 per 10⁶ cells seeded, using the standard protocol. In still other embodiments, the amount is at least 60, at least 80, at least 100, at least 120, between 80-1000, between 100-1000, between 100-800, between 80-800, or between 80-700 pg. of IL-8 per 10⁶ cells seeded.

In still other embodiments, the therapeutic moiety is SDF-1/CXCL12 (Uniprot Accession No. P48061; SDF-1 alpha, assayed herein, is a cleavage product of SDF-1). In certain embodiments, the levels of SDF-1 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of SDF-1 can be measured by methods known in the art, e.g. the described standard ELISA protocol.

Alternatively or in addition, the first and second ASC populations both secrete between 150-500 pg. (=125-250 pg./ml, as exemplified herein) of SDF-1 per 10⁶ cells seeded, using the standard protocol. In still other embodiments, the amount is at least 100, at least 150, at least 200, at least 240, at least 300, or within one of the ranges 100-1000, 100-800, 100-600, 200-1000, 200-800, 200-600, 200-500, or 300-500 pg. of SDF-1 per 10⁶ cells seeded.

In still other embodiments, the therapeutic moiety is Osteoprotegerin (Uniprot Accession No. O00300). In certain embodiments, the levels of Osteoprotegerin secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of Osteoprotegerin can be measured by methods known in the art, e.g. the described standard ELISA protocol.

Alternatively or in addition, the first and second ASC populations both secrete between 200-1400 (which equals 100-700 pg./ml; as exemplified herein) of Osteoprotegerin per 10⁶ cells seeded, using the standard protocol. In still other embodiments, the amount is at least 100, at least 150, at least 200, at least 240, at least 300, between 100-1000, between 200-2000, between 200-1600, between 200-1400, between 100-2000, between 100-1600, between 100-1400, between 150-2000, between 150-1600, or between 150-1400 pg. of Osteoprotegerin per 10⁶ cells seeded.

In still other embodiments, the therapeutic moiety is MIF (Macrophage migration inhibitory factor). In certain embodiments, the levels of MIF secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of MIF can be measured by methods known in the art, e.g. the described standard ELISA protocol.

Alternatively or in addition, the first and second ASC populations both secrete between 2000-8000 pg. (=1000-4000 pg./ml, as exemplified herein) of MIF per 10⁶ cells seeded, using the standard protocol. In still other embodiments, the amount is at least 1000, at least 1500, at least 2000, at least 2400, at least 3000; or within one of the ranges 1000-10,000, 1000-8000, 2000-16,000, 2000-14,000, 2000-12,000, or 2000-10,000 pg. of MIF per 10⁶ cells seeded.

In yet other embodiments, the therapeutic moiety is M-CSF (Accession No. P09603). In certain embodiments, the levels of M-CSF secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of M-CSF can be measured by methods known in the art, e.g. the described standard ELISA protocol.

Alternatively or in addition, the first and second ASC populations both secrete (as exemplified herein) between 300-800 pg. of M-CSF per 0.5×10⁶ cells seeded, using the standard protocol. In still other embodiments, the amount is at least 200, at least 300, at least 400, between 400-800, between 400-700, between 500-800, or between 500-700 pg. of M-CSF per 0.5×10⁶ cells seeded.

In other embodiments, the therapeutic moiety is selected from MCP-1 (CCL2), GDF-15, IL-6, IL-8, ENA78/CXCL5, eotaxin/CCL11, MCP-3 (CCL7), GM-CSF, HGF, G-CSF, IL-10, CCL5 (RANTES; Uniprot Acc. No. P13501), sICAM-1 (Acc. No. Q99930), Osteopontin, TGF-β1, IL-11, IDO (Indoleamine 2,3-dioxygenase 1; No. P14902) and PD-L1 (CD274; No. Q9NZQ7). In still other embodiments, the therapeutic moiety is a hormone, a non-limiting example of which is PGE2 (ChEMBL identifier CHEMBL548). In more specific embodiments, the amounts of the therapeutic moiety secreted by the 2 populations are within 2-fold of one another. In other embodiments, the amounts of the moiety are within 1.5 fold, 3 fold, or 5 fold of one another.

In still other embodiments, the therapeutic moiety is a pro-inflammatory factor. In certain embodiments, the moiety is secreted Beta2-M (Uniprot Accession No. P61769). In certain embodiments, the levels of Beta2-M secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of Beta2-Microglobulin can be measured by methods known in the art, e.g. the described standard ELISA protocol.

Alternatively or in addition, the first and second ASC populations both secrete between 60,000-300,000 pg. (=30,000-150,000 pg./ml, as exemplified herein) of Beta2-M per 10⁶ cells seeded, using the standard protocol. In still other embodiments, the amount is at least 50,000, at least 60,000, at least 80,000, at least 100,000; or within one of the ranges 60,000-400,000, 60,000-360,000, 60,000-240,000, 60,000-200,000, 60,000-160,000, 80,000-400,000, 80,000-360,000, 80,000-300,000, 80,000-240,000, 80,000-200,000, 80,000-160,000, 100,000-400,000, 100,000-360,000, 100,000-300,000, 100,000-240,000, 100,000-200,000, 100,000-160,000, or 110,000-150,000 pg./ml of Beta2-M per 10⁶ cells seeded.

In other embodiments, the moiety is selected from CXCL9 (Uniprot Accession No. Q07325), IL-31 (Accession No. Q6EBC2), CXCL11 (Accession No. 014625), IFN-g, and FLT-3L. In more specific embodiments, the amounts of the therapeutic moiety secreted by the 2 populations are within 2-fold of one another. In other embodiments, the amounts of the moiety are within 1.5 fold, 3 fold, or 5 fold of one another.

Also provided herein are ASCs for use in a method for treating an inflammatory disorder, said method comprising the steps of (a) administering a first pharmaceutical composition, comprising allogeneic ASC from a first donor (a first ASC population); and subsequently (b) administering a second pharmaceutical composition comprising allogeneic ASC from a second donor (a second ASC population), wherein the second donor differs from the first donor in at least one allele group of HLA-A or HLA-B; wherein the administrations are separated in time from each other by at least 7 days. Also provided is use of the described 1^{st} and 2^{nd} pharmaceutical compositions in the manufacture of a medicament for treating an inflammatory disorder. In other embodiments, there is provided an article of manufacture, comprising a packaging material and the described 1^{st} and 2^{nd} pharmaceutical compositions identified for treating an inflammatory disorder, the compositions being contained within the packaging material. In some embodiments, the indication is specified in a leaflet that is included within the article of manufacture.

In some embodiments, there is provided a method of treating a hematopoietic disorder, comprising: a) the step of administering to a subject a first pharmaceutical composition, comprising a first ASC population; and b) the step of administering to the subject, at least 7 days after step a), a second pharmaceutical composition comprising a second ASC population, wherein the second donor differs from the first donor in at least one allele group of HLA-A or HLA-B, thereby treating a hematopoietic disorder. In some embodiments, the hematopoietic disorder is selected from the disorders described in PCT Publication No. WO/2016/151476 to Zami Aberman, which is incorporated herein by reference. In other embodiments, the hematopoietic disorder is hematopoietic failure following exposure to radiation to chemotherapy, as described in PCT Publication No. WO2012/127320 to Raphael Gorodetsky and Zami Aberman.

In certain embodiments, the described first ASC population and second ASC population are derived from the same tissue, which may be, in some embodiments, placenta. In other embodiments, the tissue is adipose, or is bone marrow. In still other embodiments, the tissue is another source of ASC.

Alternatively or in addition, the ASC secrete a factor(s) that supports hematopoietic stem cell (HSC) engraftment, which is, in some embodiments, FGF-7 (Fibroblast growth factor 7; Uniprot Accession No. P21781). In certain embodiments, the levels of FGF-7 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of FGF-7 can be measured by methods known in the art, e.g. the described standard ELISA protocol.

Alternatively or in addition, the first and second ASC populations both secrete (as exemplified herein) between 300-900 pg. of FGF-7 per 0.5×10⁶ cells seeded, using the standard protocol. In still other embodiments, the amount is at least 150, at least 200, at least 300, at least 350, between 200-900, between 300-800, between 300-500, between 400-600, between 500-900, or between 500-800 pg. of FGF-7 per 0.5×10⁶ cells seeded.

In other embodiments, the factor is selected from G-CSF, GROa, IL-6, IL-8, MCP-1, ENA78, GM-CSF, Fractalkine (Uniprot Accession No. P78423), MCP-3, and LIF. In certain embodiments, the levels of the factor secreted by the 2 populations are within 2-fold of one another. In other embodiments, the secretion levels of the 2 populations are within 1.5 fold, 3 fold, or 5 fold of one another. Those skilled in the art will appreciate, in light of this disclosure, that secretion levels of these factors can be measured by methods known in the art, the standard ELISA protocol.

Alternatively or in addition, the first and second ASC populations both secrete between 2000-8000 pg. (=1000-4000 pg./ml, as exemplified herein) of MIF per 10⁶ cells seeded, using the standard protocol. In still other embodiments, the amount is at least 1000, at least 1500, at least 2000, at least 2400, at least 3000, or within one of the ranges 1000-10,000, 1000-8000, 2000-16,000, 2000-14,000, 2000-12,000, or 2000-10,000 pg. of MIF per 10⁶ cells seeded.

Alternatively or in addition, both populations of ASC induce secretion of KC ((keratinocyte chemoattractant/CXCL1; Uniprot No. P09341), IL-6, and GM-CSF in the serum and/or BM of irradiated subjects. The described ASC, in particular fetally-derived ASC, induce secretion of KC, IL-6, and GM-CSF in the serum and BM, and increase levels of RBC, WBC, and platelets, when administered to subjects with hematological deficiencies, as provided in PCT Publication No. WO/2016/151476, which is incorporated herein by reference in its entirety. In further embodiments, both ASC populations induce an amount of a cytokine selected from KC, IL-6, and GM-CSF within 2-fold of one another, when 2×10⁶ ASC are administered intramuscularly on days 1 and 5 following irradiation with an LD_{70/30} dose. In other embodiments, the levels of the cytokine induced of the 2 populations are within 1.5 fold, 3 fold, or 5 fold of one another.

Also provided herein are ASCs for use in a method for treating a hematopoietic disorder, said method comprising the steps of (a) administering a first pharmaceutical composition, comprising a first ASC population; and subsequently (b) administering a second pharmaceutical composition comprising a second ASC population, wherein the second donor differs from the first donor in at least one allele group of HLA-A or HLA-B; wherein the administrations are separated in time from each other by at least 7 days. Also provided is use of the described first and second pharmaceutical compositions in the manufacture of a medicament for treating a hematopoietic disorder. In other embodiments, there is provided an article of manufacture, comprising a packaging material and the described first and second pharmaceutical compositions identified for treating a hematopoietic disorder, the pharmaceutical compositions being contained within the packaging material. In some embodiments, the indication is specified in a leaflet that is included within the article of manufacture.

In some embodiments, there is provided a method of treating a neoplastic disorder, comprising: a) the step of administering to a subject a first pharmaceutical composition, comprising a first ASC population; and b) the step of administering to the subject, at least 7 days after step a), a second pharmaceutical composition comprising a second ASC population, wherein the second donor differs from the first donor in at least 1 allele group of HLA-A or HLA-B, thereby treating a neoplastic disorder. In some embodiments, the neoplastic disorder is selected from the tumors and neoplasms described in WO 2017/141181, which is incorporated herein by reference

In certain embodiments, the described first ASC population and second ASC population are derived from the same tissue, which may be, in some embodiments, placenta. In other embodiments, the tissue is adipose, or is bone marrow. In still other embodiments, the tissue is another source of ASC.

Alternatively or in addition, the ASC secrete a factor(s) with anti-tumor ability, which may be, in some embodiments, TRAIL (a.k.a. Tumor necrosis factor ligand superfamily member 10 or Apo-2L; Uniprot Accession no. P50591). In other embodiments, the therapeutic factor is selected from CXCL9, IL-10, IL-31, IFN-g, CXCL11, Adiponectin (Accession No. Q15848), Angiopoietin-1, and ADAM10-processed FasL form. In still other embodiments, the therapeutic factor is selected from the factors described in WO 2017/141181, which is incorporated herein by reference. In some embodiments, the described first and second ASC populations both secrete the same factor. In more specific embodiments, the amounts of the factor secreted by the 2 populations are within 2-fold of one another. In other embodiments, the amounts of the factor are within 1.5 fold, 3 fold, or 5 fold of one another.

In certain embodiments, the neoplastic disorder is triple-negative breast cancer (TNBC). In certain embodiments, the anti-TNBC moiety is selected from G-CSF, GM-CSF, IL-1RA (Accession No. P18510), Adiponectin, IL-24, Angiopoietin 1, sIL-2R (Miedel M C et al), IFN-g, IL-12p70 (the heterodimer of the IL-12A and IL-12B chains), IL-13, CXCL9/MIG, IL-33, IL-31, CXCL11, IL-15 (Acc. No. P40933), CCL3/MIP-1a (Acc. No. P10147), FLT-3L, MMP-12 (Acc. No. P39900), IL-28A, IL-28B (Acc. Nos. Q8IZJ0 and Q8IZI9, respectively), IFN-beta, ICAM-1, and IL-21. In some embodiments, the described first ASC population and second ASC populations both secrete the same factor. In more specific embodiments, the amounts of the factor secreted by the 2 populations are within 2-fold of one another. In other embodiments, the amounts of the factor are within 1.5 fold, 3 fold, or 5 fold of one another.

Also provided herein are ASCs for use in a method for treating a neoplastic disorder, said method comprising the steps of (a) administering a first pharmaceutical composition, comprising a first ASC population; and subsequently (b) administering a second pharmaceutical composition comprising a second ASC population, wherein the 2^{nd} donor differs from the 1^{st} donor in at least 1 allele group of HLA-A or HLA-B; wherein the administrations are separated in time from each other by at least 7 days. Also provided is use of the described 1^{st} and 2^{nd} pharmaceutical compositions in the manufacture of a medicament for treating a neoplastic disorder. In other embodiments, there is provided an article of manufacture, comprising a packaging material and the described first and second pharmaceutical compositions identified for treating a neoplastic disorder, the compositions being contained within the packaging material. In some embodiments, the indication is specified in a leaflet that is included within the article of manufacture.

In some embodiments, there is provided a method of treating a muscle injury, comprising: a) the step of administering to a subject a first pharmaceutical composition, comprising a first ASC population; and b) the step of administering to the subject, at least 7 days after step a), a second pharmaceutical composition comprising a second ASC population, wherein the ^{2nd} donor differs from the 1^{st} donor in at least 1 allele group of HLA-A or HLA-B, thereby treating a muscle injury. In certain embodiments, the muscle injury is a skeletal muscle injury. In more specific embodiments, the muscle injury is a post-surgical trauma. In still other embodiments, the muscle injury is any muscle injury mentioned in WO/2011/147967, which is incorporated herein by reference.

Alternatively or in addition, both ASC populations secrete an anti-fibrotic factor(s), which may be, in some embodiments, any factor mentioned herein. In other embodiments, both populations secrete factor(s) that facilitate recovery from pro-fibrotic disorders, a non-limiting example of which is pulmonary fibrosis. In certain embodiments, both populations secrete Serpin E1 (Plasminogen activator inhibitor 1; Uniprot Accession No. P05121) and uPAR (Urokinase plasminogen activator surface receptor; Accession No. Q03405), which were found to be secreted by maternal and fetal cells. In other embodiments, the described first and second ASC populations both secrete the same factor. In more specific embodiments, the amounts of the factor secreted by the 2 populations are within 2-fold of one another, using the standard protocol. In other embodiments, the amounts of the factor are within 1.5 fold, 3 fold, or 5 fold of one another.

In still other embodiments, both ASC populations secrete 2 or more, in other embodiments 3 or more, in other embodiments 4 or more, in other embodiments 5 or more, in other embodiments 6 or more, in other embodiments 7 or more, in other embodiments 8 or more, in other embodiments 9 or more, or in other embodiments 10 or more of Decorin (Uniprot Accession No. P07585), Follistatin (Acc. No. P19883), IGFBP-3 (Acc. No. P17936), IGFBP-6 (Acc. No. P24592), FLRG (Follistatin-related protein 3/FSTL3; Acc. No. 095633), Osteopontin (Acc. No. P10451), Galectin-1 (Acc. No. P09382), MCP-1, HGF, Angiopoietin 1, MMP-1 (Interstitial collagenase; Acc. No. P03956), MMP-2 (72 kDa type IV collagenase; Acc. No. P08253), MMP-10 (Stromelysin-2; Acc. No. P09238), VEGF, and TGFβ. In other embodiments, the described first ASC population and second ASC populations both secrete the same factor. In more specific embodiments, the amounts of the factor secreted by the 2 populations are within 2-fold of one another, using the standard protocol. In other embodiments, the amounts of the factor are within 1.5 fold, 3 fold, or 5 fold of one another.

In yet other embodiments, both ASC populations secrete 2 or more, in other embodiments 3 or more, in other embodiments 4 or more, in other embodiments 5 or more of TIMP1, TIMP2, MMP-1, MMP-2, and MMP-10. In other embodiments, the ASC secrete TIMP1, TIMP2, MMP-1, MMP-2, and MMP-10, which were found to be secreted by maternal cells. In still other embodiments, the ASC secrete TIMP1, TIMP2, MMP-1, and MMP-10, which were found to be secreted by fetal cells. In more specific embodiments, the amounts of the factor secreted by the 2 populations are within 2-fold of one another, using the standard protocol. In other embodiments, the amounts of the factor are within 1.5 fold, 3 fold, or 5 fold of one another

Also provided herein are ASCs for use in a method for treating a muscle injury, said method comprising the steps of (a) administering a first pharmaceutical composition, comprising allogeneic ASC from a first donor (first ASC population); and subsequently (b) administering a second pharmaceutical composition comprising allogeneic ASC from a second donor (second ASC population), wherein the second donor differs from the first donor in at least one allele group of HLA-A or HLA-B; wherein the administrations are separated in time from each other by at least 7 days. Also provided is use of the described first and second pharmaceutical compositions in the manufacture of a medicament for treating a muscle injury. In other embodiments, there is provided an article of manufacture, comprising a packaging material and the described first and second pharmaceutical compositions identified for treating a muscle injury, the pharmaceutical compositions being contained within the packaging material. In some embodiments, the indication is specified in a leaflet that is included within the article of manufacture. In certain embodiments, the described first ASC population and second ASC population are derived from the same tissue, which may be, in some embodiments, placenta. In other embodiments, the tissue is adipose, or is bone marrow. In still other embodiments, the tissue is another source of ASC.

Alternatively or in addition, the first and second ASC populations secrete one or more factors that promote muscle regeneration. In certain embodiments, the factor is Decorin, In certain embodiments, the levels of Decorin secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of Decorin can be measured by methods known in the art, e.g. the described standard ELISA protocol.

Alternatively or in addition, the first and second ASC populations both secrete between 4600-8000 pg. (=2300-4000 pg./ml (as exemplified herein) of Decorin per 10⁶ cells seeded, using the standard protocol. In still other embodiments, the 2 populations secrete at least 2000, at least 3000, at least 4000, at least 5000, at least 5600, between 100-10,000, between 1200-10,000, between 1600-10,000, between 2000-10,000, between 3000-10,000, between 4000-10,000, between 5000-10,000, between 4000-8000, between 5000-8000, or between 5600-7000 pg. of Decorin per 10⁶ cells seeded, using the standard protocol.

In other embodiments, the factor is Osteopontin. In certain embodiments, the levels of Osteopontin secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of Osteopontin can be measured by methods known in the art, e.g. the described standard ELISA protocol.

Alternatively or in addition, the first and second ASC populations both secrete between 6000-16,000 pg. (=3000-8000 pg./ml, as exemplified herein) of Osteopontin per 10⁶ cells seeded, using the standard protocol. In still other embodiments, the 2 populations secrete at least 2000, at least 3000, at least 4000, at least 5000, at least 6000, between 2000-40,000, between 2000-30,000, between 2000-24,000, between 4000-40,000, between 4000-30,000, between 4000-24,000, between 6000-40,000, between 6000-30,000, between 6000-24,000, between 8000-40,000, between 8000-30,000, or between 8000-24,000 pg. of Osteopontin per 10⁶ cells seeded

In yet other embodiments, the factor is Angiopoietin-1. In certain embodiments, the levels of Angiopoietin-1 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of Angiopoietin-1 can be measured by methods known in the art, e.g. the described standard ELISA protocol.

Alternatively or in addition, the first and second ASC populations both secrete between 200-3000 pg. (=100-1500 pg./ml, as exemplified herein) of Angiopoietin-1 per 10⁶ cells seeded, using the standard protocol. In still other embodiments, the 2 populations secrete at least 100, at least 120, at least 160, at least 200, at least 300, between 200-4000, between 300-4000, between 300-3000, between 300-2400, between 360-4000, between 360-3000, or between 360-2400 pg. of Angiopoietin-1 per 10⁶ cells seeded, using the standard protocol.

In yet other embodiments, the factor is FLRG/FSTL3 (“FLRG”). In certain embodiments, the levels of FLRG secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of FLRG can be measured by methods known in the art, e.g. the described standard ELISA protocol.

Alternatively or in addition, the first and second ASC populations both secrete between 3000-30,000 pg. (=1500-15,000 pg./ml, as exemplified herein) of FLRG per 10⁶ cells seeded, using the standard protocol. In still other embodiments, the 2 populations secrete at least 1000, at least 1200, at least 1600, at least 2000, at least 3000, at least 4000, at least 5000; or within one of the ranges 2000-40,000, 2000-30,000, 3000-40,000, 3000-24,000, 3000-20,000, 3000-18000, 4000-40,000, 4000-30,000, 5000-40,000, or 5000-30,000 pg. of FLRG per 10⁶ cells seeded, using the standard protocol.

In yet other embodiments, the factor is Galectin-1. In certain embodiments, the levels of Galectin-1 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of Galectin-1 can be measured by methods known in the art, e.g. the described standard ELISA protocol.

Alternatively or in addition, the first and second ASC populations both secrete between 6000-30,000 pg. (=3000-15,000 pg./ml, as exemplified herein) of Galectin-1 per 10⁶ cells seeded, using the standard protocol. In still other embodiments, the 2 populations secrete at least 1000, at least 1500, at least 2000, at least 3000, at least 4000, at least 5000, at least 6000, or within one of the ranges 6000-40,000, 6000-36,000, 6000-20,000, 2000-40,000, 2000-30,000, 2000-24,000, 2000-20,000, 3000-40,000, 3000-30,000, 3000-24,000, 3000-20,000, 4000-40,000, 4000-30,000, 4000-24,000, or 4000-20,000 pg. of Galectin-1 per 10⁶ cells seeded, using the standard protocol.

In still other embodiments, the factor secreted by the first and second ASC populations is selected from Follistatin, IGFBP-3, IGFBP-6, MCP-1, HGF, MMP-1, MMP-2, MMP-10, VEGF, and TGF-β. In certain embodiments, the levels of the factor secreted by the 2 populations are within 2-fold of one another, using the standard protocol. In other embodiments, the secretion levels of the 2 populations are within 1.5 fold, 3 fold, or 5 fold of one another.

In yet other embodiments, the factor secreted by the 1^{st} and 2^{nd} ASC populations promotes extracellular matrix remodeling. In certain embodiments, the factor is a TIMP (Metalloproteinase inhibitor), which is, in some embodiments, TIMP1; or in other embodiments TIMP2; or in other embodiments TIMP3 (Uniprot Accession Nos. P01033, P16035, and P35625, respectively).

In certain embodiments, the levels of TIMP1 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of TIMP1 can be measured by methods known in the art, e.g. the described standard ELISA protocol.

Alternatively or in addition, the first and second ASC populations both secrete between 60,000-200,000 pg. (=30,000-100,000 pg./ml, as exemplified herein) of TIMP1 per 10⁶ cells seeded, using the standard protocol. In still other embodiments, the 2 populations secrete at least 30,000, at least 40,000, at least 60,000, at least 70,000; or within one of the ranges 40,000-200,000, 40,000-160,000, 40,000-150,000, 50,000-200,000, 50,000-160,000, 50,000-150,000, 60,000-400,000, 60,000-300,000, 60,000-160,000, or 60,000-150,000 pg. of TIMP1 per 10⁶ cells seeded, using the standard protocol.

In yet other embodiments, the factor is MMP-2. In certain embodiments, the levels of MMP-2 secreted by the 2 populations are within 2-fold of one another. In other embodiments, the secretion levels of the 2 populations are within 1.5 fold, 3 fold, or 5 fold of one another. Those skilled in the art will appreciate, in light of the present disclosure, that secretion levels of MMP-2 can be measured by methods known in the art, e.g. the described standard ELISA protocol.

Alternatively or in addition, the first and second ASC populations both secrete between 200,000-600,000 pg. (=100,000-300,000 pg./ml, as exemplified herein) of MMP-2 per 10⁶ cells seeded, using the standard protocol. In still other embodiments, the 2 populations secrete at least 200,000, at least 240,000, at least 300,000, at least 360,000, at least 400,000; or within one of the ranges 200,000-800,000, 300,000-600,000, 300,000-560,000, 360,000-600,000, 360,000-560,000, 400,000-600,000, or 400,000-560,000 pg. MMP-2/10⁶ cells seeded, using the standard protocol.

In other embodiments, the factor that promotes extracellular matrix remodeling is selected from TIMP2, MMP-1, MMP-2, and MMP-10. In certain embodiments, the levels of the factor secreted by the 2 populations are within 2 fold of one another, using the standard protocol. In other embodiments, the secretion levels are within 1.5 fold, 3 fold, or 5 fold of one another.

In some embodiments, there is provided a method of treating an orthopedic condition, comprising the steps of: a) administering to a subject a first pharmaceutical composition, comprising a first ASC population; and b) administering to the subject, at least 7 days after step a), a second pharmaceutical composition comprising a second ASC population, wherein the 2nd donor differs from the 1st donor in at least 1 allele group of HLA-A or HLA-B, thereby treating an orthopedic condition. In certain embodiments, the condition is an injured bone, tendon or ligament. In other embodiments, the condition is an inflamed bone, tendon or ligament. In still other embodiments, the condition is selected from Achilles tendinosis, adhesive capsulitis, ankle syndesmosis, inflamed carpal tunnel sheath (flexor retinaculum), and rotator cuff tendinitis. Each condition represents a separate embodiment. Also provided herein are ASCs for use in a method for treating an orthopedic condition. In certain embodiments, both ASC populations secrete an anti-fibrotic factor(s), which may be, in some embodiments, any factor mentioned herein. In other embodiments, the described first and second ASC populations both secrete the same factor. In more specific embodiments, the amounts of the factor secreted by the 2 populations are within 2-fold of one another, using the standard protocol. In other embodiments, the amounts of the factor are within 1.5 fold, 3 fold, or 5 fold of one another. In yet other embodiments, the factor secreted by the 1^{st} and 2^{nd} ASC populations promotes extracellular matrix remodeling.

In some embodiments, there is provided a method of treating preeclampsia, comprising: a) the step of administering to a subject a first pharmaceutical composition, comprising a first ASC population; and b) the step of administering to the subject, at least 7 days after step a), a second pharmaceutical composition comprising a second ASC population, wherein the second donor differs from the first donor in at least one allele group of HLA-A or HLA-B, thereby treating preeclampsia. In certain embodiments, the preeclampsia is selected from early-onset preeclampsia, severe preeclampsia, and late-onset preeclampsia. Alternatively or in addition, the ASC secrete a factor(s) that stimulates angiogenesis, which may be, in some embodiments, selected from VEGF, Angiogenin, PDGF, and IL-8. Other relevant embodiments are described in WO 2014/037863, which is incorporated herein by reference. In other embodiments, the described first and second ASC populations both secrete the same factor. In more specific embodiments, the amounts of the factor secreted by the 2 populations are within 2-fold of one another, using the standard protocol. In other embodiments, the amounts of the factor are within 1.5 fold, 3 fold, or 5 fold of one another. Alternatively or in addition, the described first AS and second ASC population are derived from the same tissue, which may be, in some embodiments, placenta. In other embodiments, the tissue is adipose, or is bone marrow. In still other embodiments, the tissue is another source of ASC.

Also provided herein are ASCs for use in a method for treating preeclampsia, said method comprising the steps of: (a) administering a first pharmaceutical composition, comprising a first ASC population; and subsequently (b) administering a second pharmaceutical composition comprising a second ASC population, wherein the second donor differs from the first donor in at least one allele group of HLA-A or HLA-B; wherein the administrations are separated in time from each other by at least 7 days. Also provided is use of the described 1^{st} and 2^{nd} compositions in the manufacture of a medicament for treating preeclampsia. In other embodiments, there is provided an article of manufacture, comprising a packaging material and the described first and second pharmaceutical compositions identified for treating preeclampsia, the pharmaceutical compositions being contained within the packaging material. In some embodiments, the indication is specified in a leaflet that is included within the article of manufacture.

In some embodiments, there is provided a method of treating respiratory distress syndrome, or in other embodiments acute lung injury (ALI), comprising the steps of: a) administering to a subject a first pharmaceutical composition, comprising allogeneic ASC from a first donor; and b) administering to the subject, at least 7 days after step a), a second pharmaceutical composition comprising allogeneic ASC from a second donor, wherein the second donor differs from the first donor in at least one allele group of HLA-A or HLA-B, thereby treating respiratory distress syndrome or ALI. In certain embodiments, the respiratory distress syndrome is acute respiratory distress syndrome (ARDS), which may be, in more specific embodiments, pulmonary ARDS, or extrapulmonary ARDS. In still further embodiments, the ARDS comprises pulmonary edema, which may be, in more specific embodiments, noncardiogenic pulmonary edema; or in other embodiments arterial hypoxemia; or in other embodiments, a combination thereof. In other embodiments, the respiratory distress syndrome is infant respiratory distress syndrome (IRDS). Alternatively or in addition, the ASC secrete immunoregulatory factor(s), anti-fibrotic factor(s), or factor(s) that promotes extracellular matrix formation, which may be, in some embodiments, any factor mentioned herein. Other relevant embodiments are described in WO 2018/185584, which is incorporated herein by reference. In other embodiments, the described first and second ASC populations both secrete the same factor, using the standard protocol. In other embodiments, the amounts of the factor secreted by the 2 populations are within 2-fold of one another. In other embodiments, the amounts of the factor are within 1.5 fold, 3 fold, or 5 fold of one another.

In some embodiments, there is provided a method of reducing morbidity, or in other embodiments mortality in a subject exposed to a vesicant, or in other embodiments in a subject exposed to an organophosphate agent, comprising: a) the step of administering to a subject a first pharmaceutical composition, comprising a first ASC population; and b) the step of administering to the subject, at least 7 days after step a), a second pharmaceutical composition comprising a second ASC population, wherein the second donor differs from the first donor in at least one allele group of HLA-A or HLA-B, thereby treating exposure to a vesicant or an organophosphate agent. In certain embodiments, the organophosphate agent is selected from butyrylcholinesterase inhibitors and acetylcholinesterase inhibitors, more specifically organophosphorus agents and carbamates. Alternatively or in addition, the ASC secrete a factor(s) selected from G-CSF (Granulocyte colony-stimulating factor; Uniprot Accession No. P09919); GM-CSF (Granulocyte-macrophage colony-stimulating factor; Acc. No. P04141); GROa/CXCL1; IL-6; IL-8; MCP-1, MCP-3 (Monocyte chemoattractant proteins 1 and 3/Acc. Nos. P13500 and P80098, respectively), ENA78 (CXCLS; Acc. No. P42830); LIF (Leukemia inhibitory factor); EPO (Erythropoietin; Acc. No. P01588), IL-3 (interleukin-3; Acc. No. P08700), and SCF. Other relevant embodiments are described in U.S. Provisional Patent Ser. No. 62/723,026, which is incorporated herein by reference. In other embodiments, the described 1^{st} and 2^{nd} ASC populations both secrete the same factor. In more specific embodiments, the amounts of the factor secreted by the 2 populations are within 2-fold of one another, using the standard protocol. In other embodiments, the amounts of the factor are within 1.5 fold, 3 fold, or 5 fold of one another. Alternatively or in addition, the 1^{st} and 2^{nd} ASC population are derived from the same tissue, which may be, in some embodiments, placenta. In other embodiments, the tissue is adipose, or is bone marrow. In still other embodiments, the tissue is another source of ASC.

Also provided herein are ASCs for use in a method for treating exposure to a vesicant or an organophosphate agent, said method comprising the steps of (a) administering a first pharmaceutical composition, comprising a first ASC population; and subsequently (b) administering a second pharmaceutical composition comprising a second ASC population, wherein the 2^{nd} donor differs from the 1^{st} donor in at least one allele group of HLA-A or HLA-B; wherein the administrations are separated in time from each other by at least 7 days. Also provided is use of the described first and second pharmaceutical compositions in the manufacture of a medicament for treating exposure to a vesicant or an organophosphate agent. In other embodiments, there is provided an article of manufacture, comprising a packaging material and the described first and second pharmaceutical compositions identified for treating exposure to a vesicant or an organophosphate agent, the pharmaceutical compositions being contained within the packaging material. In some embodiments, the indication is specified in a leaflet that is included within the article of manufacture.

In still other embodiments, there is a provided a method of treating a chronic disorder that requires multiple administrations of ASC (e.g. 2, 3, 4, 5, or at least 2, 3, 4, or 5), comprising administration of multiple ASC populations, e.g. as described herein. In certain embodiments, the chronic disorder is selected from a chronic ischemic disorder, a chronic hematopoietic disorder, a chronic neurodegenerative disorder, a chronic inflammatory disorder, or a chronic orthopedic condition, each of which represents a separate embodiment. Also provided herein are ASCs for use in a method for treating a chronic disorder.

Reference to ASC “from” or “derived from” a donor is intended to encompass cells removed from or otherwise obtained from the donor, followed by optional steps of ex-vivo cell culture, expansion, and/or other treatments to improve the therapeutic efficacy of the cells; and/or combination with pharmaceutical excipients. Those skilled in the art will appreciate that the aforementioned optional steps will not alter the HLA genotype of the ASC, absent intentional modification of the HLA genotype (e.g. using CRISPR-mediating editing or the like). Cell populations with an intentionally modified HLA genotype are not intended to be encompassed. ASC populations that contain a mixture cells from more than one donor are also not intended to be encompassed.

As will be appreciated by those skilled in the art, the HLA system or complex is a gene complex encoding the major histocompatibility complex (MHC) proteins in humans These cell-surface proteins are involved in regulation of the immune system in humans. The HLA gene complex resides on a 3-Mbp stretch within chromosome 6p21. HLA genes are highly polymorphic. HLAs encoding MHC class I proteins (“class I HLA's”) present peptides from inside the cell, while class II HLA's present external peptides.

There are 3 major MHC class I genes, HLA-A, HLA-B, and HLA-C; and 3 minor class I genes, HLA-E, HLA-F and HLA-G. β2-microglobulin binds with major and minor gene subunits to produce a heterodimer.

There are 3 major (DP, DQ and DR) and 2 minor (DM and DO) MHC class II proteins encoded by the HLA. The class II MHC proteins combine to form heterodimeric (αβ) protein receptors that are typically expressed on the surface of antigen-presenting cells.

HLA alleles are often named according to a multi-partite system, where the letter prefix (e.g. “HLA-A”) denotes the locus, followed by an asterisk; followed by the “allele group” number; followed by the specific HLA protein number; followed by a number used to denote silent DNA mutations in a coding region; followed by, lastly, a number used to denote DNA mutations in a non-coding region (Robinson J et al). For example, in the hypothetical allele “HLA-A*02:07:01:03”, the allele group number is 02; 07 is the specific HLA protein number; 01 describes a pattern of silent DNA mutations in the coding regions; and 03 describes a pattern of DNA mutations in non-coding regions. “Mutations” in this regard refers to variations relative to the founder (initially identified) allele in the allele group. Typically, an allele group corresponds to a particular encoded serological antigen, while specific HLA proteins within an allele group exhibit relatively minor differences. In certain embodiments, an “antigenic” difference refers to a different allele group, while an “allelic” difference refers to a different HLA protein within the same allele group.

HLA typing at each locus, may be, in some embodiments, low resolution, or “first-level field” typing, by reference to the two digits preceding the first separator, or antigen level typing, e.g. A*02 in the above example. In various other embodiments, the typing is at “intermediate-level” resolution, i.e. second-level field, e.g. HLA-A*02:07, or in other embodiments, third-level field, e.g. HLA-A*02:07:01. In other embodiments, the typing is “allele level typing”, using all digits in the first, second, third and fourth fields, e.g. HLA-A*02:07:01:03.

Allele groups are clustered into “supertypes” which have similar peptide binding repertoires. Examples of HLA-A supertypes are 1, 2, 3, and 24, and examples of HLA-B supertypes are 7, 27, 44, 58, and 62. Typically, an allele supertype corresponds to a particular encoded serological antigen.

As provided herein (Example 5), subjects were treated for intermittent claudication by 2 administrations of placental ASC, which were from 1 of 3 populations (the P041011, P090112, or P270114 population; also referred to as “04”, “09”, or “27”, respectively). Some subjects received the same population twice, while others received 2 different populations in the 2 administrations. Subjects treated with a dose of 300×10⁶ cells per timepoint exhibited a superior therapeutic effect when given placental ASC from 2 different donors. In other words, serial administration of ASC from different donors is shown herein to be more efficacious than repeat administration of ASC from the same donor.

As indicated below in Table 1, 04, 09, and 27 each differ from the other batches by at least 1 of 2 alleles for HLA-A. For each combination, there is at least 1 difference in the HLA-A superfamilies, and 0 or 1 differences in the HLA-B superfamilies. 04 and 27, for example, do not share any HLA-A or HLA-B allele groups. The HLA-A alleles of 04 and 27 have no common supertypes, while their HLA-B alleles have one common supertype.

TABLE 1
HLA-A, HLA-B, HLA-C, HLA-DR, and HLA-DQ profiles
of P041011, P090112, and P270114. The supertypes
of all the HLA-A and HLA-B alleles listed have
been experimentally established (Sidney J et al).

| | P04 allele/ | P09 allele/ | P27 allele/ |
|---|---|---|---|
| Population | supertype | supertype | supertype |
| HLA-A #1 | A11:01/A03 | A11:01/A03 | A01:01/A01 |
| HLA-A#2 | A68:02/A02 | A24:02/A24 | A23:01/A24 |
| HLA-B#1 | B14:02/B27 | B44:02/B44 | B44:03/B44 |
| HLA-B#2 | B52:01/B62 | B52:01/B62 | B15:01/B62 |
| HLA-C#1 | C*08:02 | C*05:01 | C*04:01 |
| HLA-C#2 | C*12:02 | C*12:02 | C*12:03 |
| HLA-DRB1#1 | DRB1-01:02 | DRB1-12:01 | DRB1-07:01 |
| HLA-DRB1#2 | DRB1-13:02 | DRB1-15:02 | DRB1-14:01 |
| HLA-DQB1#1 | DQB1*05:01 | DQB1*03:01 | DQB1*02:02 |
| HLA-DQB1#2 | DQB1*06:09 | DQB1*06:01 | DQB1*05:03 |

Number of shared superfamilies

| Combination | HLA-A alleles | HLA-B alleles |
|---|---|---|
| 04 vs. 09 | 1/2 | 1/2 |
| 04. vs. 27 | 0/2 | 1/2 |
| 09 vs. 27 | 1/2 | 2/2 |

Without wishing to be bound by theory, given the importance of HLA-A, HLA-B, and HLA-DR in transplant compatibility, and the lack of significant surface expression of HLA-DR in placental ASC, the present inventors propose that the additional efficacy conferred by serial treatment with P041011 and P090112 may be connected to difference(s) in their HLA-A and HLA-B alleles.

Those skilled in the art will appreciate that the protein sequences of HLA-A*11:01, HLA-A*01:01, HLA-A*68:02, HLA-A*24:02, and HLA-A*23:01 (SEQ ID Nos. 1-5, respectively) are set forth in GenBank Nucleotide Accession Nos. AY786587, EU445470, U03861, M64740, and M64742.1. The protein sequences of HLA-B*14:02, HLA-B*44:02, HLA-B*15:01, HLA-B*52:01, and HLA-B*44:03 (SEQ ID Nos. 6-10) are set forth in Accession Nos. M24032, M24038, U03859, AH002881.2, and LN877362.2.

As set forth in Sidney J et al, and without wishing to be bound by theory, the main binding energy for HLA class I peptides is typically provided by the interaction of the position 2 and the C-terminal (anchor) residues of the peptide, with the B and F binding pockets of the MHC molecule, respectively. Residues 7, 9, 24, 34, 45, 63, 66, 67, 70, and 99 are considered, in some embodiments, as delineating the B pocket (which engages position 2), with residues 9, 45, 63, 66, 67, 70, and 99 considered, in other embodiments, as key residues. The residues considered, in some embodiments, as delineating the F pocket (which engages the C-terminal residue) are 74, 77, 80, 81, 84, 95, 97, 114, 116, 123, 133, 143, 146, and 147, with residues 77, 80, 81 and 116 considered, in other embodiments, as key residues.

It will also be appreciated that alleles of a given supertype will have similar binding preferences at position 2 and the anchor residues of the bound peptide. By way of example, HLA-A*24:02, and HLA-A*23:01 both have residues S, M, E, K, V, H, and F at key B-pocket positions 9, 45, 63, 66, 67, 70, and 99, respectively. These 2 alleles further have Y, A, and V at other B-pocket positions 7, 24, and 34, respectively. As a result, they favor aromatic and aliphatic residues (F, W, Y, L, I, V, M, or Q) at position 2. Additionally, these 2 alleles both have residues N, I, A, and Y at key F-pocket positions 77, 80, 81 and 116, and they both have residues D, Y, L, M, H, Y, W, T, K, and W at other F-pocket positions 74, 84, 95, 97, 114, 123, 133, 143, 146, and 147, respectively. As a result, they favor large hydrophobic residues (F, L, I, M) at the C-terminal anchor position. Since HLA-A*24:02, and HLA-A*23:01 have the same binding specificities, they belong to the same superfamily, A24. (It should be noted that some alleles within a superfamily may exhibit slight variations. For example, B*27:03 favors hydrophobic and basic residues at the C-terminus, while B*27:09 favors large hydrophobic C-terminal residues; both alleles belong to B27). The binding preferences of the alleles of 04, 09, and 27 are set forth in Table 2.

TABLE 2
Binding preferences for the 04, 09,
and 27 HLA-A and HLA-B alleles.

| Allele(s) | Superfam. | Position 2 preference | C-terminal preference |
|---|---|---|---|
| A*11:01 | A03 | Small & aliphatic | Basic (R, H, or K) |
| | | (A, T, S, V, L, I, | |
| | | M, or Q) | |
| A*01:01 | A01 | Small & aliphatic | Aromatic (F, W, or Y) |
| A*68:02 | A02 | Small & aliphatic | Aliphatic (L, I, V, |
| | | | M, or Q) |
| A*24:02 | A24 | Aromatic & aliphatic | Large hydrophobic |
| A*23:01 | | (F, W, Y, L, I, V, | (F, L, I, or M) |
| | | M, or Q) | |
| B*14:02 | B27 | Basic | Hydrophobic |
| | | | (L, I, V, M, F, W, |
| | | | Y, or A) |
| B*44:02 | B44 | Acidic (D or E) | Hydrophobic |
| B*44:03 | | | |
| B*15:01 | B62 | Aliphatic | Large hydrophobic |
| B*52:01 | | | |

Reference to a second donor “differ/differs/differing” from a first donor in at least one allele group of HLA-A or HLA-B denotes that the DNA of the second donor comprises at least one HLA-A or HLA-B allele belonging to an allele group not represented in the alleles of the first donor. (Typically [except in the case of a homozygous first donor], the DNA of the first donor will also comprise at least one HLA-A or HLA-B allele belonging to an allele group not represented in the alleles of the second donor). Similarly, a second donor “differs from” a first donor in at least one allele supertype if the DNA of the second donor comprises at least one HLA-A or HLA-B allele belonging to a supertype not represented in the alleles of the first donor. These terms are intended to be used analogously in various contexts herein, except where indicated otherwise.

In other embodiments, the second donor in the described therapeutic methods and compositions differs from the first donor in at least one allele group of HLA-A. In still other embodiments, the second donor differs from the first donor in at least one allele group of HLA-B.

In yet other embodiments, the second donor differs from the first donor in both HLA-A allele groups of or, in other embodiments, in both HLA-B allele groups; or, in other embodiments, at least one allele group of each of HLA-A and HLA-B.

In other embodiments, the second donor differs from the first donor in at least one HLA-A allele supertype or, in other embodiments, at least one HLA-B allele supertype.

In still other embodiments, the second donor differs from the first donor in at least two allele supertypes of HLA-A or HLA-B, which may be, in more specific embodiments, an HLA-A allele supertype, an HLA-B allele supertype, or a combination thereof.

In certain embodiments, the HLA-A alleles of the first and second donor differ from each other in at least one superfamily; while in other embodiments, they differ from each other in both superfamilies. Alternatively or additionally, the HLA-B alleles of the first and second donor differ from each other in at least one superfamily; while in other embodiments, they differ from each other in both superfamilies. In still other embodiments, the HLA-A alleles of the first and second donor differ from each other in at least one superfamily, while the HLA-B superfamilies do not differ. In yet other embodiments, the HLA-A alleles differ from each other in both superfamilies, while the HLA-B superfamilies do not differ.

Alternatively or in addition, the second donor differs from the first donor in at least one allele group of HLA-DR, or in other embodiments, in 2 HLA-DR allele groups.

Alternatively or in addition, the second donor differs from the first donor in at least one allele group of HLA-C, or in other embodiments, in 2 HLA-C allele groups. In still other embodiments, the second donor exhibits at least an allelic difference from the first donor in at least one allele of HLA-C, or in other embodiments, in both HLA-C alleles.

Alternatively or in addition, the second donor differs from the first donor in at least one allele group of HLA-DQ, or in other embodiments, in 2 HLA-DQ allele groups. In still other embodiments, the second donor exhibits at least an allelic difference from the first donor in at least one allele of HLA-DQ, or in other embodiments, in both HLA-DQ alleles.

Alternatively or in addition, the second donor differs from the first donor in at least one allele group of Histo-blood group ABO system transferase (“ABO”; Uniprot Accession No. P16442), or in other embodiments, in 2 ABO allele groups. In still other embodiments, the second donor exhibits at least an allelic difference from the first donor in at least one allele of ABO, or in other embodiments, in both ABO alleles.

Certain herein-described methods comprise a step of administering a second pharmaceutical composition comprising allogeneic ASC from a second donor (“second administration”). This step is, in some embodiments, performed between 2-52 weeks after administration of the first ASC population. In other embodiments, the second administration is performed between 4-24 weeks after the first administration. In still other embodiments, the interval is between 3-52, 4-26, 5-26, 6-20, 6-18, 6-15, 6-10, 3-20, 3-15, 3-10, 4-12, 4-20, 5-18, 6-16, 8-16, 10-16, or 8-12 weeks. In yet other embodiments, the second administration is performed between 53-104 weeks after the first administration.

Alternatively or in addition, the second administration is followed by an additional step of administering to the subject, at least 7 days after the second administration, a third pharmaceutical composition comprising allogeneic ASC derived from a third donor, wherein the third donor differs from both the first donor and the second donor in at least one allele group of HLA-A or HLA-B (i.e. has an allele group not represented in either the first or second donor), which is, in various embodiments, an allele of HLA-A or HLA-B. In other embodiments, the third donor differs from both the first donor and the second donor in at least two allele groups of HLA-A or HLA-B, which are, in various embodiments, an allele of HLA-A, HLA-B, or a combination thereof. Recitation herein of administration of a third pharmaceutical composition does not preclude subsequent additional administration(s) of pharmaceutical composition(s), which, in some embodiments, may comprise allogeneic ASC derived from additional donor(s), besides those already administered to the subject.

In various embodiments, engraftment of the described cells in the host is not required for the cells to exert the described therapeutic effects, each of which is considered a separate embodiment. In other embodiments, engraftment is required for the cells to exert the therapeutic effect(s). For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days.

ASC and Sources Thereof

In certain embodiments, each of the described ASC populations are plastic adherent under standard culture conditions, express the surface molecules CD105, CD73 and CD90, and do not express CD45, CD34, CD14 or CD11b, CD79α, CD19 and HLA-DR.

ASC can be propagated, in some embodiments, by using a combination of 2D and 3D substrates. Conditions for propagating adherent cells on 2D and 3D substrates are further described hereinbelow and in the Examples section which follows.

In other embodiments, each of the described ASC populations are placenta-derived. Except where indicated otherwise herein, the terms “placenta”, “placental tissue”, and the like refer to any portion of the placenta. Placenta-derived adherent cells may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer [e.g., phosphate-buffered saline (PBS) or Hank's buffer]. In certain embodiments, the placental tissue from which cells are harvested includes at least one of the chorionic and decidua regions of the placenta, or, in still other embodiments, both the chorionic and decidua regions of the placenta. More specific embodiments of chorionic regions are chorionic mesenchymal and chorionic trophoblastic tissue. More specific embodiments of decidua are decidua basalis, decidua capsularis, and decidua parietalis.

Single-cell suspensions can be made, in other embodiments, by treating the tissue with a digestive enzyme (see below) or/and physical disruption, a non-limiting example of which is mincing and flushing the tissue parts through a nylon filter or by gentle pipetting (e.g. Falcon, Becton, Dickinson, San Jose, Calif.) with washing medium. In some embodiments, the tissue treatment includes use of a DNAse, a non-limiting example of which is Benzonase from Merck.

Placental cells may be obtained, in various embodiments, from a full-term or pre-term placenta. In some embodiments, residual blood is removed from the placenta before cell harvest. This may be done by a variety of methods known to those skilled in the art, for example by perfusion. The term “perfuse” or “perfusion” as used herein refers to the act of pouring or passaging a fluid over or through an organ or tissue. In certain embodiments, the placental tissue may be from any mammal, while in other embodiments, the placental tissue is human. A convenient source of placental tissue is a post-partum placenta (e.g., less than 10 hours after birth), however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to the surface of an adherent material to thereby isolate adherent cells. In some embodiments, the donor is 35 years old or younger, while in other embodiments, the donor may be any woman of childbearing age.

Reference herein to “growth” of a population of cells is intended to be synonymous with expansion of a cell population. In certain embodiments, ASC (which may be, in certain embodiments, placental ASC), are expanded without substantial differentiation. In various embodiments, the described expansion is on a 2D substrate, on a 3D substrate, or a 2D substrate, followed by a 3D substrate.

In still other embodiments, each of the described ASC populations is a placental cell population that is a mixture of fetal-derived placental ASC (also referred to herein as “fetal ASC” or “fetal cells”) and maternal-derived placental ASC (also referred to herein as “maternal ASC” or “maternal cells”), where a majority of the cells are maternal cells. In more specific embodiments, the mixture contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.95%, at least 99.96%, at least 99.97%, at least 99.98%, or at least 99.99% maternal cells, or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9%, 99-99.9%, 99.2-99.9%, 99.5-99.9%, 99.6-99.9%, 99.7-99.9%, or 99.8-99.9% maternal cells.

Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g. a Y chromosome in the case of a male fetus).

In other embodiments, each ASC population is a placental cell population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, each ASC population is a placental cell population that is a mixture of fetal and maternal cells, where a majority of the cells are fetal. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, 99% or 100% of the cells are fetal. Expression of CD200, as measured by flow cytometry, using an isotype control to define negative expression, can be used as a marker of fetal cells under some conditions. In yet other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal.

In more specific embodiments, the mixture contains 20-80% fetal cells; 30-80% fetal cells; 40-80% fetal cells; 50-80% fetal cells; 60-80% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

In yet other embodiments, each of the described ASC populations is adipose-derived. As used herein, the phrase “adipose tissue” refers to a connective tissue which comprises fat cells (adipocytes). Those skilled in the art will appreciate that adipose tissue-derived ASC may be extracted, in various embodiments, by a variety of methods known to those skilled in the art, for example those described in U.S. Pat. No. 6,153,432, which is incorporated herein by reference. The adipose tissue may be derived, in other embodiments, from omental/visceral, mammary, gonadal, or other adipose tissue sites. In some embodiments, adipose cells can be isolated by liposuction. In other embodiments, ASC may be derived from adipose tissue by treating the tissue with a digestive enzyme (non-limiting examples of which are collagenase, trypsin, dispase, hyaluronidase or DNAse); and ethylenediaminetetra-acetic acid (EDTA). The cells may be, in some embodiments, subjected to physical disruption, for example using a nylon or cheesecloth mesh filter. In other embodiments, the cells are subjected to differential centrifugation directly in media or over a Ficoll or Percoll or other particulate gradient (see U.S. Pat. No. 7,078,230, which is incorporated herein by reference).

In still other embodiments, the ASC are derived from BM. Those skilled in the art will appreciate that BM-ASC can be obtained by Ficoll® extraction to remove red blood cells. During this process, fresh BM is diluted 5:14 in isolation buffer (PBS+2% FBS+2 mM EDTA) and spun down at 300×g for 30 minutes. The interface layer containing the mononuclear cells is then removed and resuspended in 40 ml cold isolation buffer, which is then centrifuged again at 300×g for 10 minutes. The resulting cells are then optionally resuspended in expansion medium and plated on a tissue culture apparatus.

In still other embodiments, each of the described ASC populations includes mesenchymal stromal cells (MSC). These cells are, in some embodiments, isolated from bone marrow. In further embodiments, the cells are human MSC, as defined by The Mesenchymal and Tissue Stem Cell Committee of the International Society for Cellular Therapy (Dominici et al, 2006), based on the following 3 criteria: 1. Plastic-adherence when maintained in standard culture conditions (a minimal essential medium plus 20% fetal bovine serum (FBS)). 2. Expression of the surface molecules CD105, CD73 and CD90, and lack of expression of CD45, CD34, CD14 or CD11b, CD79α or CD19 and HLA-DR. 3. Ability to differentiate into osteoblasts, adipocytes and chondroblasts in vitro.

In still other embodiments, the ASC are derived from peripheral blood; umbilical cord blood; synovial fluid; synovial membranes; spleen; thymus; mucosa (for example nasal mucosa); limbal stroma; ligament (e.g. periodontal ligament); dermis; scalp; hair follicles, testicles; embryonic yolk sac; muscle tissue; or amniotic fluid.

In some embodiments, each of the described ASC populations are allogeneic human ASC.

Additional Markers and Characteristics of ASC

Alternatively or additionally, each of the populations of ASC used in the described methods and compositions (which may be, in various embodiments, 2 populations, 3 populations, or more) expresses a marker or a collection of markers (e.g. surface markers) characteristic of MSC or mesenchymal-like stromal cells. In some embodiments, each ASC population expresses some or all of the following markers: CD105 (UniProtKB Accession No. P17813), CD29 (Acc. No. P05556), CD44 (Acc. No. P16070), CD73 (Acc. No. P21589), and CD90 (Acc. No. P04216). In some embodiments, each population does not express some or all of the following markers: CD3 (Acc. Nos. P09693 [gamma chain], P04234 [delta chain], P07766 [epsilon chain], and P20963 [zeta chain]), CD4 (Acc. No. P01730), CD11b (Acc. No. P11215), CD14 (Acc. No. P08571), CD19 (Acc. No. P15391), and/or CD34 (Acc. No. P28906). In more specific embodiments, each population also lacks expression of CD5 (Acc. No. P06127), CD20 (Acc. No. P11836), CD45 (Acc. No. P08575), CD79-alpha (Acc. No. B5QTD1), CD80 (Acc. No. P33681), and/or HLA-DR (Accession Nos. P04233 [gamma chain], P01903 [alpha chain], and P01911 [beta chain]). The aforementioned, non-limiting marker expression patterns were found in certain maternal placental cell populations that were expanded on 3D substrates. Those skilled in the art will appreciate that the presence of complex antigens such as CD3 and HLA-DR may be detected by antibodies recognizing any of their component parts, such as, but not limited to, those mentioned herein.

“Positive” expression of a marker indicates a value higher than the range of the main peak of a fluorescence-activated cell sorting (FACS) isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

In further embodiments, each population of ASC (e.g. placental ASC) expresses one or more markers that are not expressed (or at least not highly expressed) in BM-MSC. In certain embodiments, the expressed markers are selected from any combination of CD46 (Uniprot Acc. No. P15529), CD59 (No. P15529), CD61 (P05106), CD140b (P09619), CD144 (P33151), and CD150 (Q13291). Alternatively or in addition, the cells do not express one or more markers that are expressed in BM-MSC. In certain embodiments, the non-expressed markers are selected from any combination of CD62P (No. P16109), CD109 (Q6YHK3), CD112 (Q92692), and CD154 (P29965). In yet other embodiments, the cells do not express CD9 (No. P21926) at high levels; and/or do express CD55 (P21926) at high levels. See Winkler T et al. Uniprot entries were accessed on Jun. 10, 2019.

In some embodiments, each population of ASC possesses a marker phenotype that is distinct from bone marrow-mesenchymal stem cells (BM-MSC). In certain embodiments, each ASC population is positive for expression of CD10 (which occurs, in some embodiments, in both maternal and fetal ASC); is positive for expression of CD49d (which occurs, in some embodiments, at least in maternal ASC); is positive for expression of CD54 (which occurs, in some embodiments, in both maternal and fetal ASC); is bimodal, or in other embodiments positive, for expression of CD56 (which occurs, in some embodiments, in maternal ASC); and/or is negative for expression of CD106. Except where indicated otherwise, bimodal refers to a situation where a significant percentage (e.g. at least 20%) of a population of cells express a marker of interest, and a significant percentage do not express the marker.

In certain embodiments, over 90% of the cells in each ASC population are positive for CD29, CD90, and CD54. In other embodiments, over 85% of the described cells are positive for CD29, CD73, CD90, and CD105. In yet other embodiments, less than 3% of the described cells are positive for CD14, CD19, CD31, CD34, CD39, CD45RA (an isotype of CD45), HLA-DR, Glycophorin A, and CD200; less than 6% of the cells are positive for GlyA; and less than 20% of the cells are positive for SSEA4. In more specific embodiments, over 90% of the described cells are positive for CD29, CD90, and CD54; and over 85% of the cells are positive for CD73 and CD105. In still other embodiments, over 90% of the described cells are positive for CD29, CD90, and CD54; over 85% of the cells are positive for CD73 and CD105; less than 6% of the cells are positive for CD14, CD19, CD31, CD34, CD39, CD45RA, HLA-DR, GlyA, CD200, and GlyA; and less than 20% of the cells are positive for SSEA4. The aforementioned, non-limiting marker expression patterns were found in certain maternal placental cell populations that were expanded on 3D substrates.

In other embodiments, each of CD73, CD29, and CD105 is expressed by more than 90% of the cells in each ASC population; and over 90% (or in other embodiments, over 95%, or in other embodiments, over 98%) of the cells in each ASC population do not differentiate into adipocytes, under conditions where MSC would differentiate into adipocytes. In some embodiments, as provided herein, the conditions are incubation of adipogenesis induction medium, for example a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days (“standard adipogenesis induction conditions”). In yet other embodiments, for each ASC population, each of CD34, CD45, CD19, CD14 and HLA-DR is expressed by less than 3% of the cells; and the cells do not differentiate into adipocytes, after incubation under the standard conditions. In other embodiments, each of CD73, CD29, and CD105 is expressed by more than 90% of the cells, each of CD34, CD45, CD19, CD14 and HLA-DR is expressed by less than 3% of the cells; and the cells do not differentiate into adipocytes, after incubation under the standard conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days (“modified adipogenesis induction conditions”). The aforementioned solutions will typically contain cell culture medium such as DMEM+10% serum or the like, as will be appreciated by those skilled in the art. The aforementioned, non-limiting phenotypes and marker expression patterns were found in certain maternal placental cell populations that were expanded on 3D substrates, as described herein. Placental cells expanded as described herein are resistant to adipogenesis, as described in WO 2016/098061, in the name of Esther Lukasiewicz Hagai et al, published on Jun. 23, 2016, which is incorporated herein by reference

In still other embodiments, the majority, in other embodiments over 60%, over 70%, over 80%, or over 90% of the ASC in each of the ASC populations express CD29, CD73, CD90, and CD105. In yet other embodiments, less than 20%, 15%, or 10% of the described cells express CD3, CD4, CD34, CD39, and CD106. In yet other embodiments, less than 20%, 15%, or 10% of the described cells highly express CD56. In various embodiments, each ASC population is less than 50%, less than 40%, less than 30%, less than 20%, or less than 10%, or less than 5% positive for CD200. In other embodiments, each ASC population is more than 50%, more than 60%, more than 70%, more than 80%, more than 90%, more than 95%, more than 97%, more than 98%, more than 99%, or more than 99.5% positive for CD200. In certain embodiments, more than 50% of the cells express, or in other embodiments highly express, CD141 (thrombomodulin; UniProt Accession No. P07204), or in other embodiments SSEA4 (stage-specific embryonic antigen 4, an epitope of ganglioside GL-7 (IV³ NeuAc2→3 Ga1GB4); Kannagi R et al), or in other embodiments both markers. Alternatively or in addition, more than 50% of the cells express HLA-A2 (Accession No. P01892). The aforementioned, non-limiting marker expression patterns were found in certain fetally-derived placental cell populations that were expanded on 3D substrates.

In other embodiments, each of CD29, CD73, CD90, and CD105 is expressed by more than 80% of the ASC in each of the populations; and over 90% (or in other embodiments, over 95%, or over 98%) of the cells in each population do not differentiate into osteocytes, after incubation for 17 days with a solution containing 0.1 mcM dexamethasone, 0.2 mM ascorbic acid, and 10 mM glycerol-2-phosphate, in plates coated with vitronectin and collagen (“standard osteogenesis induction conditions”). In yet other embodiments, each of CD34, CD39, and CD106 is expressed by less than 10% of the cells; less than 20% of the cells highly express CD56; and the cells do not differentiate into osteocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56, and/or the cells do not differentiate into osteocytes, after incubation under the standard conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen (“modified osteogenesis induction conditions”). The aforementioned solutions will typically contain cell culture medium such as DMEM+10% serum or the like, as will be appreciated by those skilled in the art. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetal tissue-derived placental cell populations that were expanded on 3D substrates, as provided herein. Placental cells expanded as described herein are resistant to osteogenesis, as described in WO 2016/098061, which is incorporated herein by reference.

In other embodiments, each of CD29, CD73, CD90, and CD105 is expressed by more than 80% of each of the ASC populations; and over 90% (or in other embodiments, over 95%, or over 98%) of the cells in each population do not differentiate into adipocytes, under standard adipogenesis induction conditions. In yet other embodiments, each of CD34, CD39, and CD106 is expressed by less than 10% of the cells; less than 20% of the cells highly express CD56; and the cells do not differentiate into adipocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56; and the cells do not differentiate into adipocytes, after incubation under the standard conditions. In still other embodiments, the described modified adipogenesis induction conditions are used. In still other embodiments, over 90% of the cells in each population do not differentiate into either adipocytes or osteocytes under the aforementioned standard conditions. In yet other embodiments, over 90% of the cells in each population do not differentiate into either adipocytes or osteocytes under the modified conditions. The aforementioned solutions will typically contain cell culture medium such as DMEM+10% serum or the like, as will be appreciated by those skilled in the art. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetal tissue-derived placental cell populations that were expanded on 3D substrates.

In other embodiments, each of CD73, CD29, and CD105 is expressed by more than 90% of each ASC population; and each ASC population inhibits proliferation of LPS-stimulated T cells. In yet other embodiments, each of CD34, CD19, and CD14 is expressed by less than 3% of the cells; and the cells inhibit T cell proliferation. In other embodiments, each of CD73, CD29, and CD105 is expressed by more than 90% of the cells, each of CD34, CD19, and CD14 is expressed by less than 3% of the cells; and the cells inhibit T cell proliferation. In certain embodiments, the inhibition of T cell proliferation, expressed as the percent decrease in T cell proliferation relative to a control, varies less than 20% (or in other embodiments, less than 10%).

In yet other embodiments, the ASC secrete factors that induce proliferation and/or migration of myoblasts. Appropriate conditions for a myoblast proliferation assay are described in Winkler T et al, and include preparation of CM (e.g. by seeding 1 million ASC in 2 ml DMEM medium; replacing the medium with EBM-2 medium and incubating for an additional 24 hours) and incubating myoblasts (e.g. C2C12 cells) in the CM. Factors that induce myoblast migration include Follistatin, IGFBP-3, Osteopontin, and Galectin-1.

In other embodiments, the cells in each ASC populations exhibit a spindle shape when cultured under 2D conditions.

According to some embodiments, each ASC populations express CD200, while in other embodiments, the populations lack expression of CD200. In still other embodiments, less than 30%, 25%, 20%, 15%, 10%, 8%, 6%, 5%, 4%, 3%, or 2%, 1%, or 0.5% of the adherent cells express CD200. In yet other embodiments, greater than 70%, 75%, 80%, 85%, 90%, 92%, 94%, 95%, 96%, 97%, 98%, 99%, or 99.5% of the adherent cells express CD200.

In still other embodiments, the cells in each ASC population are allogeneic, or in other embodiments, the cells are autologous. In other embodiments, the cells are fresh or, in other embodiments, frozen (for example, cryopreserved).

In various embodiments, any of the embodiments of surface marker expression and other characteristics may be freely combined with the described embodiments of cytokine expression.

Use of Serum-Deficient Medium and Serum-Replacement Medium for Cell Expansion

In other embodiments, the described cell population is produced by expanding a cell population (for example, a population of placental adherent cells) in a medium that contains less than 5% animal serum. In other embodiments, the medium contains less than 4%; less than 3%; less than 2%; less than 1%; less than 0.5%; less than 0.3%; less than 0.2%; or less than 0.1% animal serum. In other embodiments, the medium does not contain animal serum. In other embodiments, the medium is a defined medium to which no serum has been added. Low-serum and serum-free media are collectively referred to as “serum-deficient medium/media”.

Those skilled in the art will appreciate that reference herein to animal serum includes serum from a variety of species, provided that the serum stimulates expansion of the ASC population. In certain embodiments, the serum is mammalian serum, non-limiting examples of which are human serum, bovine serum (e.g. fetal bovine serum and calf bovine serum), equine serum, goat serum, and porcine serum.

In certain embodiments, the serum-deficient medium is supplemented with factors intended to stimulate cell expansion in the absence of serum. Such medium is referred to herein as serum-replacement medium or SRM, and its use, for example in cell culture and expansion, is known in the art, and is described, for example, in Kinzebach et al.

In other embodiments, the serum-deficient medium contains one or more growth factors. In certain embodiments, the growth factors, individually or, in other embodiments collectively, induce cell expansion in culture. In other embodiments, the growth factors, individually or, in other embodiments collectively, induce cell expansion in culture without differentiation.

In more specific embodiments, the factor(s) contained in the serum-deficient medium is selected from a FGF, TGF-beta (Uniprot Accession no. P01137), transferrin (e.g. serotransferrin or lactotransferrin; Accession nos. P02787 and P02788), insulin (Accession no. P01308), EGF (epidermal growth factor; Accession no. P01133), and/or PDGF (platelet-derived growth factor, including any combination of subunits A and B; Accession nos. P04085 and P01127), each of which represents a separate embodiment. A non-limiting example of PDGF is PDGF-BB.

Except where indicated otherwise, reference herein to a protein includes all its isoforms functional fragments thereof, and mimetics thereof. Such reference also includes homologues from a variety of species, provided that the protein acts on the target cells in a similar fashion to the homologue from the same species as the target cells. For example, if human cells are being expanded, reference to bFGF would also include any non-human bFGF that stimulates proliferation of human cells. Those skilled in the art will appreciate that, even in the case of human cells, the aforementioned proteins need not be human proteins, since many non-human (e.g. animal) proteins are active on human cells. Similarly, the use of modified proteins that have similar activity to the native forms falls within the scope of the described methods and compositions.

The FGF (fibroblast growth factor) family includes a number of proteins that are described in Imamura. A non-limiting example is bFGF.

In other embodiments, the serum-deficient medium comprises an FGF and TGF-beta. In still other embodiments, the medium comprises an FGF, TGF-beta, and PDGF. In more specific embodiments, the medium further comprises transferrin, insulin, or both transferrin and insulin. Alternatively or in addition, the medium further comprises oleic acid.

In still other embodiments, the serum-deficient medium comprises an FGF and EGF. In still other embodiments, the medium further comprises transferrin, insulin, or both transferrin and insulin.

SRM formulations include MSC Nutristem® XF (Biological Industries); Stempro® SFM and Stempro® SFM-XF (Thermo Fisher Scientific); PPRF-msc6; D-hESF10; TheraPEAK™ MSCGM-CDTM (Lonza, cat. no. 190632); and MesenCult-XF (Stem Cell Technologies, cat. no. 5429). The StemPro® media contain PDGF-BB, bFGF, and TGF-β, and insulin (Chase L G et al).

In certain embodiments, the described SRM comprises bFGF (basic fibroblast growth factor, also referred to as FGF-2), TGF-β (TGF-β, including all isotypes, for example TGFβ1, TGFβ2, and TGFβ3), or a combination thereof. In other embodiments, the SRM comprises bFGF, TGF-β, and PDGF.

Other embodiments of incubation of ASC in serum-deficient medium are described in PCT Appl. No. PCT/IB2019/052569, to Lior Raviv et al, which is incorporated herein by reference.

Incubation with Pro-Inflammatory Cytokines

In certain embodiments, each of the described ASC populations has been incubated with pro-inflammatory cytokines. Reference herein to one or more “pro-inflammatory” cytokines, or “inflammatory cytokines”, which is used interchangeably, implies the presence of at least one cytokine that mediates an inflammatory response in a mammalian host, for example a human host. A non-limiting list of cytokines are Interferon-gamma (IFN-gamma; UniProt Accession No. P01579), IL-22 (No. Q9GZX6), Tumor Necrosis Factor-alpha (TNF-alpha; No. P01375), IFN-alpha, IFN-beta (No. P01574), IL-1alpha (No. P01583), IL-1beta (No. P01584), IL-17 (No. Q5QEX9), IL-23 (No. Q9NPF7), IL-17A (No. Q16552), IL-17F (No. Q96PD4), IL-21 (No. Q9HBE4), IL-13 (No. P35225), IL-5 (No. P05113), IL-4 (No. P05112), IL-33 (No. 095760), IL-1RL1 (No. Q01638), TNF-Beta (No. P01374), IL-11 (No. P20809), IL-9 (No. P15248), IL-2 (No. P60568), Tumor Necrosis Factor-Like Ligand (TL1A; a.k.a. TNF ligand superfamily member 15; No. O95150), IL-12 (Nos. P29459 and P29460 for alpha- and beta subunits), and IL-18 (No. Q14116). Additional cytokines include (but are not limited to) Leukemia inhibitory factor (LIF), oncostatin M (OSM; No. P13725), ciliary neurotrophic factor (CNTF; No. P26441), and IL-8.

Except where indicated otherwise, reference to a cytokine or other protein is intended to include all isoforms of the protein. For example, IFN-alpha includes all the subtypes and isoforms thereof, such as but not limited to IFN-alpha 17, IFN-alpha 4, IFN-alpha 7, IFN-alpha 8, and IFN-alpha 110. Some representative UniProt identifiers for IFN-alpha are P01571, P05014, P01567, P32881, and P01566. Those skilled in the art will appreciate that, even in the case of human cells, the aforementioned cytokines need not be human cytokines, since many non-human (e.g. animal) cytokines are active on human cells. Similarly, the use of modified cytokines that have similar activity to the native forms falls within the scope of the described embodiments.

In certain embodiments, one or more of the cytokines is TNF-alpha. In more specific embodiments, the TNF-alpha may be the only cytokine present, or, in other embodiments, may be present together with 1, 2, 3, 4, 5, 6, 1-2, 1-3, 1-4, 1-5, or 1-6, or more than 6 added inflammatory cytokines.

In some embodiments, TNF-alpha is present together with IFN-gamma These two cytokines may be the only 2 added cytokines, or, in other embodiments, present with additional proinflammatory cytokines.

In certain embodiments, one or more of the cytokines is IFN-gamma. In more specific embodiments, the IFN-gamma may be the only cytokine present, or, in other embodiments, may be present together with 1, 2, 3, 4, 5, 6, 1-2, 1-3, 1-4, 1-5, or 1-6, or more than 6 added cytokines.

In certain embodiments, the target cell concentration is reached by perfusing the cells in cytokine-containing medium. In other embodiments, perfusion is subsequently continued with cytokine-containing medium, but the rate of perfusion is adjusted to maintain homeostasis of one or more other parameters, for example glucose concentration, pH, dissolved oxygen concentration, or the like.

Those skilled in the art will appreciate that animal sera and other sources of growth factors are often included in growth media. In some cases, animal sera may contain inflammatory cytokines, which, in general, will not generally be present in large amounts. Some preparations utilize sera that are treated, for example with charcoal, so as to remove most or all of the cytokines present. In any event, reference herein to “added cytokines”, “medium containing cytokines”, or the like, does not encompass the presence of cytokines present in animal sera that is customarily included in the medium.

It will also be appreciated that in certain embodiments, when the described ASC are intended for administration to a human subject, the cells and the culture medium (e.g., with the above-described medium additives) are substantially xeno-free, i.e., devoid of any animal contaminants For example, the culture medium can be supplemented with a serum-replacement, human serum and/or synthetic or recombinantly produced factors.

Other embodiments of incubation of ASC with pro-inflammatory cytokines are described in PCT Publ. No. WO2017/212309, to Eytan Abraham et al, which is incorporated herein by reference.

Additional Aspects of Methods for Expansion and Preparation of ASC

In certain embodiments, each of the described ASC populations have been subject to a 3D incubation, as described further herein. In more specific embodiments, the ASC have been incubated in a 2D adherent-cell culture apparatus, prior to the step of 3D culturing. In some embodiments, cells (which have been extracted, in some embodiments, from placenta, from adipose tissue, etc.) are then subjected to prior step of incubation in a 2D adherent-cell culture apparatus, followed by the described 3D culturing steps.

The terms “two-dimensional culture” and “2D culture” refer to a culture in which the cells are exposed to conditions that are compatible with cell growth and allow the cells to grow in a monolayer. An apparatus suitable for such are is referred to as a “2D culture apparatus”. Such apparatuses will typically have flat growth surfaces (also referred to as a “two-dimensional substrate(s)” or “2D substrate(s)”), in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (also referred to as a “three-dimensional substrate” or “3D substrate”), in some embodiments comprising an adherent material, which is present in the 3D culture apparatus, e.g. the bioreactor. Certain, non-limiting embodiments of 3D culturing conditions suitable for expansion of ASC are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. Other embodiments include Matrigel™, an extra-cellular matrix component (e.g., Fibronectin, Chondronectin, Laminin), and a collagen. In more particular embodiments, the material may be selected from a polyester and a polypropylene. Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids.

In other embodiments, the length of 3D culturing is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14 doublings, in other embodiments 5-13 doublings, in other embodiments 5-12 doublings, in other embodiments 5-11 doublings, in other embodiments 5-10 doublings, in other embodiments 6-15 cell doublings, in other embodiments 6-14 doublings, in other embodiments 6-13 doublings, or in other embodiments 6-12 doublings, in other embodiments 6-11 doublings, or in other embodiments 6-10 doublings.

In certain embodiments, 3D culturing can be performed in a 3D bioreactor. In some embodiments, the 3D bioreactor comprises a container for holding medium and a 3D attachment substrate disposed therein, and a control apparatus, for controlling pH, temperature, and oxygen levels and optionally other parameters. The terms attachment substrate and growth substrate are interchangeable. In certain embodiments, the attachment substrate is in the form of carriers, which comprise, in more specific embodiments, a surface comprising a synthetic adherent material. Alternatively or in addition, the bioreactor contains ports for the inflow and outflow of fresh medium and gases. Except where indicated otherwise, the term “bioreactor” excludes decellularized organs and tissues derived from a living being.

Examples of bioreactors include, but are not limited to, a continuous stirred tank bioreactor, a CelliGen Plus® bioreactor system (New Brunswick Scientific (NBS) and a BIOFLO 310 bioreactor system (New Brunswick Scientific (NBS).

As provided herein, a 3D bioreactor is capable, in certain embodiments, of 3D expansion of ASC under controlled conditions (e.g. pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, N.J.). Additional bioreactors that may be used, in some embodiments, are stationary-bed bioreactors; and air-lift bioreactors, where air is typically fed into the bottom of a central draught tube flowing up while forming bubbles, and disengaging exhaust gas at the top of the column. Additional possibilities are cell-seeding perfusion bioreactors with polyactive foams [as described in Wendt, D. et al., Biotechnol Bioeng 84: 205-214, (2003)] and radial-flow perfusion bioreactors containing tubular poly-L-lactic acid (PLLA) porous scaffolds [as described in Kitagawa et al., Biotechnology and Bioengineering 93(5): 947-954 (2006). Other bioreactors which can be used are described in U.S. Pat. Nos. 6,277,151; 6,197,575; 6,139,578; 6,132,463; 5,902,741; and 5,629,186, which are incorporated herein by reference. A “stationary-bed bioreactor” refers to a bioreactor in which the cellular growth substrate is not ordinarily lifted from the bottom of the incubation vessel in the presence of growth medium. For example, the substrate may have sufficient density to prevent being lifted and/or it may be packed by mechanical pressure to present it from being lifted. The substrate may be either a single body or multiple bodies. Typically, the substrate remains substantially in place during the standard perfusion rate of the bioreactor. In certain embodiments, the substrate may be lifted at unusually fast perfusion rates, for example greater than 200 rpm.

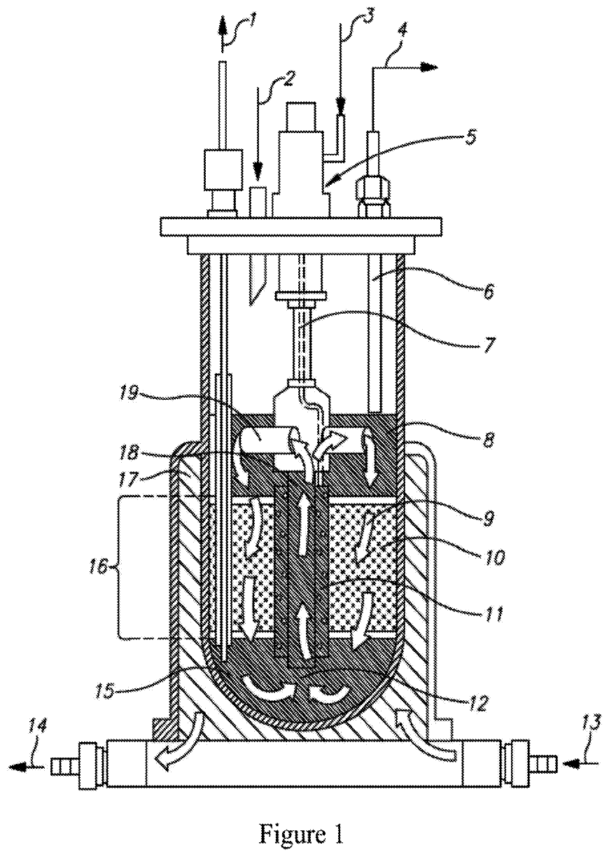

Another exemplary, non-limiting bioreactor, the Celligen 310 Bioreactor, is depicted in FIG. 1. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow initial stiffing rate is used to promote cell attachment, then the stiffing rate is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3) , which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or both together. Non-limiting examples of microcarriers that are available commercially include alginate-based (GEM, Global Cell Solutions), dextran-based (Cytodex®, GE Healthcare), collagen-based (Cultispher®, Percell™), and polystyrene-based (SoloHill® Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen.

In other embodiments, cells are produced using a packed-bed spinner flask. In more specific embodiments, the packed bed may comprise a spinner flask and a magnetic stirrer. The spinner flask may be fitted, in some embodiments, with a packed bed apparatus, which may be, in more specific embodiments, a fibrous matrix; or in more specific embodiments, a non-woven fibrous matrix. In other embodiments, the fibrous matrix comprises polyester, or comprises at least about 50% polyester. In still other embodiments, the non-woven fibrous matrix comprises polyester, or comprises at least about 50% polyester.

In still other embodiments, the matrix is similar to the Celligen™ Plug Flow bioreactor which is, in certain embodiments, packed with Fibra-cel® carriers (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the stirring speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700 mg\liter, between 450-650 mg\liter, between 475-625 mg\liter, between 500-600 mg\liter, or between 525-575 mg\liter. In yet other embodiments, at the end of the culture process, carriers are removed from the packed bed, washed with isotonic buffer, and processed or removed from the carriers by agitation and/or enzymatic digestion.

In certain embodiments, the bioreactor is seeded at a concentration of between 10,000-2,000,000 cells/ml of medium; or, in other embodiments, 20,000-2,000,000, 30,000-1,500,000, 40,000-1,400,000, 50,000-1,300,000, 60,000-1,200,000, or 70,000-1,100,000 cells/ml.

In still other embodiments, between 1-20×10⁶ cells per gram (gr) of carrier (substrate) are seeded; or, in other embodiments, 1.5-20×10⁶, 3-12×10⁶, or 4-7×10⁶, cells/gr carrier.

In certain embodiments, the harvest from the bioreactor is performed when at least about 10%, 12%, 14%, 16%, 18%, 20%, 22%, 24%, or 30% of the cells are in the S and G2/M phases (collectively), as can be assayed by various methods known in the art, for example FACS detection. Typically, in the case of FACS, the percentage of cells in S and G2/M phase is expressed as the percentage of the live cells, after gating for live cells, for example using a forward scatter/side scatter gate.

In other embodiments, incubation of ASC may comprise microcarriers, which may, in certain embodiments, be inside a bioreactor. Microcarriers are known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.), Superbeads (commercially available from Flow Labs, Inc.), and DE-52 and DE-53 (commercially available from Whatman, Inc.). In certain embodiments, the ASC may be incubated in a 2D apparatus, for example tissue culture plates or dishes, prior to incubation in microcarriers. In other embodiments, the ASC are not incubated in a 2D apparatus prior to incubation in microcarriers. In certain embodiments, the microcarriers are packed inside a bioreactor.

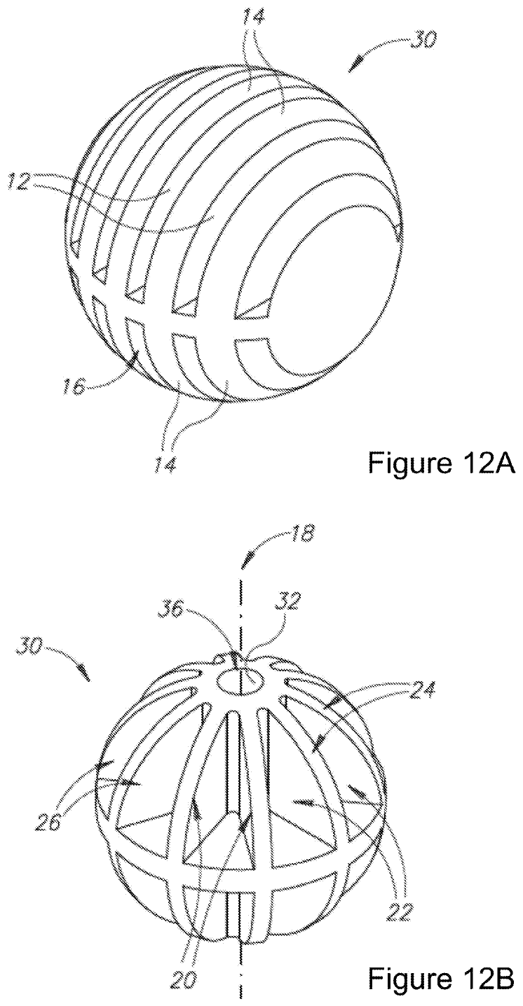

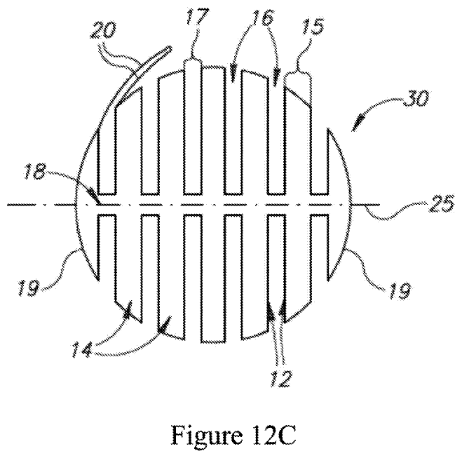

In some embodiments, with reference to FIGS. 12A-B, and as described in WO/2014/037862, published on Mar. 13, 2014, which is incorporated herein by reference, grooved carriers 30 are used for proliferation and/or incubation of each ASC population. In various embodiments, the carriers may be used following a 2D incubation (e.g. on culture plates or dishes), or without a prior 2D incubation. In other embodiments, incubation on the carriers may be followed by incubation on a 3D substrate in a bioreactor, which may be, for example, a packed-bed substrate or microcarriers; or incubation on the carriers may not be followed by incubation on a 3D substrate. Carriers 30 can include multiple two-dimensional (2D) surfaces 12 extending from an exterior of carrier 30 towards an interior of carrier 30. As shown, the surfaces are formed by a group of ribs 14 that are spaced apart to form openings 16, which may be sized to allow flow of cells and culture medium (not shown) during use. With reference to FIG. 12C, carrier 30 can also include multiple 2D surfaces 12 extending from a central carrier axis 18 of carrier 30 and extending generally perpendicular to ribs 14 that are spaced apart to form openings 16, creating multiple 2D surfaces 12. In some embodiments, carriers 30 are “3D bodies” as described in WO/2014/037862; the contents of which relating to 3D bodies are incorporated herein by reference.

In certain embodiments, the described carriers (e.g. grooved carriers) are used in a bioreactor. In some, the carriers are in a packed conformation.

In certain embodiments, further steps of purification or enrichment for ASC may be performed. Such methods include, but are not limited to, cell sorting using markers for ASC and/or, in various embodiments, mesenchymal stromal cells or mesenchymal-like ASC. Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

In more particular embodiments, cells may be removed from a 3D matrix while the matrix remains within the bioreactor. In certain embodiments, at least about 10%, 20%, or 30% of the cells are in the S and G2/M phases (collectively), at the time of harvest from the bioreactor.

In certain embodiments, the harvesting process comprises vibration or agitation, for example as described in PCT International Application Publ. No. WO 2012/140519, which is incorporated herein by reference. In certain embodiments, during harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator.

Those skilled in the art will appreciate that a variety of isotonic buffers may be used for washing cells and similar uses. Hank's Balanced Salt Solution (HBSS; Life Technologies) is only one of many buffers that may be used.

Non-limiting examples of base media useful in 2D and 3D culturing include Minimum Essential Medium Eagle, F10(HAM), F12 (HAM), Dulbecco's Modified Eagle Medium (DMEM), and others described in WO 2018/185584, which is incorporated herein by reference. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

The various media described herein, i.e. the 2D growth medium and the 3D growth medium, may be independently selected from each of the described embodiments relating to medium composition. In various embodiments, any medium suitable for growth of cells in a standard tissue apparatus and/or a bioreactor may be used.

Pharmaceutical Compositions

The described ASC can be administered as a part of a pharmaceutical composition, e.g., that further comprises one or more pharmaceutically acceptable carriers. Hereinafter, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are propylene glycol, saline, emulsions and mixtures of organic solvents with water. In some embodiments, the pharmaceutical carrier is an aqueous solution of saline.

In other embodiments, compositions are provided herein that comprise ASC in combination with an excipient, e.g., a pharmacologically acceptable excipient. In further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. In more specific embodiments, DMSO may be present at a concentration of 2-5%; or, in other embodiments, 5-10%; or, in other embodiments, 2-10%, 3-5%, 4-6%; 5-7%, 6-8%, 7-9%, 8-10%. DMSO, in other embodiments, is present with a carrier protein, a non-limiting example of which is albumin, e.g. human serum albumin The cells may be any embodiment of ASC mentioned herein, each of which is considered a separate embodiment.

Provided in addition are pharmaceutical compositions, comprising placental ASC, in the absence of non-placental cell types. Also provided are pharmaceutical compositions, comprising ASC, in the absence of cell types other than ASC.

Since non-autologous cells may in some cases induce an immune reaction when administered to a subject, several approaches may be utilized according to the methods provided herein to reduce the likelihood of rejection of non-autologous cells. In some embodiments, these approaches include suppressing the recipient immune system. In some embodiments, this may be done regardless of whether the ASC themselves engraft in the host. For example, the majority of the cells may, in various embodiments, not survive after engraftment for more than 3 days, more than 4 days, more than 5 days, more than 6 days, more than 7 days, more than 8 days, more than 9 days, more than 10 days, or more than 14 days.

Examples of immunosuppressive agents that may be used in the methods and compositions provided herein include, but are not limited to, methotrexate, cyclophosphamide, cyclosporine, cyclosporine A, chloroquine, hydroxychloroquine, sulfasalazine (sulphasalazopyrine), gold salts, D-penicillamine, leflunomide, azathioprine, anakinra, infliximab (REMICADE), etanercept, TNF-alpha blockers, biological agents that antagonize one or more inflammatory cytokines, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, and tramadol.

One may, in various embodiments, administer the pharmaceutical composition in a systemic manner (as detailed hereinabove). Alternatively, one may administer the pharmaceutical composition locally, for example, via injection of the pharmaceutical composition directly into an exposed or affected tissue region of a patient. In other embodiments, the cells are administered intravenously (IV), subcutaneously (SC), by the intraosseous route (e.g. by intraosseous infusion), or intraperitoneally (IP), each of which is considered a separate embodiment. In other embodiments, the ASC or composition is administered intramuscularly; while in other embodiments, the ASC or composition is administered systemically. In this regard, “intramuscular” administration refers to administration into the muscle tissue of a subject; “subcutaneous” administration refers to administration just below the skin; “intravenous” administration refers to administration into a vein of a subject; “intraosseous” administration refers to administration directly into bone marrow; and “intraperitoneal” administration refers to administration into the peritoneum of a subject. In still other embodiments, the cells are administered intratracheally, intrathecally, by inhalational, or intranasally. In certain embodiments, lung-targeting routes of administration may utilize cells encapsulated in liposomes or other barriers to reduce entrapment within the lungs.

In other embodiments, for injection, the described cells may be formulated in aqueous solutions, e.g. in physiologically compatible buffers such as Hank's solution, Ringer's solution, or physiological salt buffer, optionally in combination with medium containing cryopreservation agents.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

The data obtained from these in vitro and cell culture assays and animal studies can be used in formulating a range of dosage for use in human. The dosage may vary depending upon the dosage form employed and the route of administration utilized. The exact formulation, route of administration and dosage can be, in some embodiments, chosen by the individual physician in view of the patient's condition.

A typical dosage of the described ASC used alone ranges, in some embodiments, from about 75-500 million per dosing day. In certain embodiments, 100-400 million ASC are administered by intramuscular (IM) injection(s). In other embodiments, 100-300 million, 125-400 million, 150-400 million, 175-400 million, 200-400 million, 250-400 million, 300-400 million, 250-350 million, or 200-400 million ASC are administered by IM injection(s) per dosing day. In more specific embodiments, at least 2 doses are administered. In other embodiments, 2-10, 2-8, 2-5, 2-4, 2-3, or 2 doses are administered. In still other embodiments, at least 2 doses are administered, each dose originating from a different placental donor.

Alternatively or in addition, each dose is administered in a series of 1, 2, 3, 4, 5, 6, 7, 8, 9, 10, 11, 12, 13, 14, 15, 1-10, 1-15, 1-20, 2-10, 2-15, 2-20, 3-20, 4-20, 5-20, 5-25, 5-30, 5-40, or 5-50 injections.

In certain embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

In other embodiments, the described ASC are suitably formulated as a pharmaceutical composition which can be suitably packaged as an article of manufacture. Such an article of manufacture comprises a packaging material which comprises a label describing a use in treating a disease or disorder or therapeutic indication that is mentioned herein. In other embodiments, a pharmaceutical agent is contained within the packaging material, wherein the pharmaceutical agent is effective for the treatment of a disorder or therapeutic indication that is mentioned herein. In some embodiments, the pharmaceutical composition is frozen.

It is clarified that each embodiment of the described ASC may be freely combined with each embodiment relating to a therapeutic method or pharmaceutical composition.

Subjects

In certain embodiments, the subject treated by the described methods and compositions is a human; for example, a human having a peripheral artery disease, e.g. CLI or IC. In more specific embodiments, the subject is contraindicated for surgical revascularization, or in other embodiments, for both surgical and endovascular revascularization. Those skilled in the art will appreciate that contraindications for surgical revascularization include comorbidities, for example, diabetes mellitus (DM), coronary artery disease (CAD), chronic renal failure, which may be, in certain embodiments, end-stage renal disease (ESRD) on hemodialysis. In certain embodiments, the subject has 2 or more, or in other embodiments, 3 or more, of the aforementioned comorbidities. In other embodiments, the contraindications include age >70 years, unavailability of suitable vein grafts, the absence of a landing zone for distal bypass, foot infection in the site of potential anastomosis (e.g. the dorsalis pedis or the peronea), previous failed bypass. It will also be appreciated that impaired renal function is a contraindication for angioplasty (endovascular revascularization).

In certain embodiments, the human subject treated by the described methods and compositions is suffering from a hematopoietic disorder, a neurodegenerative disorder, an inflammatory disorder, an orthopedic condition, or a neoplasm. In some embodiments, the subject is a pediatric subject, for example a subject up to 1 year in age. In other embodiments, the subject is an elderly subject, for example a subject over 60, over 65, over 70, over 75, over 80, 60-85, 65-85, or 70-85 years in age. In other embodiments, the subject is 1-20 years, 20-40 years, or 40-60 years in age. In other embodiments, the subject may be an animal. In some embodiments, treated animals include domesticated animals and laboratory animals, e.g., non-mammals and mammals, for example non-human primates, rodents, pigs, rabbits, dogs, and cats. In certain embodiments, the subject may be administered with additional therapeutic agents or cells.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including ASC. In another aspect, the kits and articles of manufacture may comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Culturing and Production of Adherent Placental Cells

Placenta-derived cell populations containing over 90% maternally-derived cells were prepared as described in Example 1 of International Patent Application WO 2016/098061, in the name of Esther Lukasiewicz Hagai et al, published on Jun. 23, 2016, which is incorporated herein by reference in its entirety.

To detach the cells from the carriers, carriers were incubated with trypsin solution for 4 minutes, with oscillating mixing at 5 Hz, as described in PCT International Application Publ. No. WO 2012/140519. The medium was drained into a harvest bag, containing FBS (final concentration 10%), and the carriers were washed with isotonic solution, with oscillating mixing at 5-Hz frequency, and the cell suspension was drained into the harvest bag.

Afterwards, cells were suspended and washed in suspension solution (5% w/v human serum albumin [HSA] in isotonic solution), then adjusted to 10-20×10⁶ cells/ml, in isotonic solution with 10% DMSO v/v and 5% HSA w/v. The vials were gradually chilled and stored in a gas-phase liquid nitrogen freezer.

### Example 2: Intermediate Cell Stock Production in Serum-Free Medium

Methods

The procedure included periodic testing of the medium for sterility and contamination.

Step 1-1—Extraction and Plating of Adherent Stromal Cells (ASC's)

Placentas were obtained from donors up to 35 years old, who were pre-screened and determined to be negative for hepatitis B, hepatitis C, HIV-1 and HIV-2, HTLV-1 and HTLV-2, and syphilis. The donor placenta was maintained sterile and cooled.

Within 36 hours of the delivery, the placenta (apart from the amnion and chorion) was placed with the maternal side facing upwards and minced. Pieces were washed with isotonic buffer +gentamicin, then incubated for 1-3 hours with collagenase and DNAse in isotonic buffer. DMEM with 10% filtered FBS, L-Glutamine, and gentamicin was added, and cells were filtered through a sterile stainless-steel sieve and centrifuged. The cells were suspended in culture medium, seeded in flasks, and incubated at 37° C. in a humidified tissue culture incubator with 5% CO₂.

After 2 days, cells were washed with PBS, and CellStart™ cell attachment solution and StemPro® MSC SFM XenoFree medium (serum-free and xeno-free culture medium [SFM-XF]) (ThermoFisher Scientific, catalog no. A10675-01; hereinafter “StemPro® medium”) were added.

### Step 1-2—Initial Culturing

Cells were cultured for 2 additional passages (typically 4-10 population doublings after the first passage) in StemPro® medium+CellStart™. When reaching 60-90% confluence, cells were detached using trypsin, centrifuged, and seeded at 3.16±0.5×10³ cells/cm² in tissue culture flasks.

### Step 1-3—Cell Concentration, Washing, Formulation, Filling and Cryopreservation

The cell suspension from the final passage was centrifuged and suspended in culture medium at 20-40×10⁶ cells/milliliter (mL), then adjusted to 10% DMSO, 40% FBS, and 50% DMEM, the temperature was reduced in a controlled rate freezer, and cells were stored in a liquid nitrogen freezer to produce the ICS.

Results

Cell characteristics of several batches were assessed (Table 3).

TABLE 3
Characteristics of placental cells expanded in SF medium.
PDL refers to population doubling level-in this case,
the number of doublings since passage 1.

| | | | Total growth | cell size | |
|---|---|---|---|---|---|
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 20 | 18.9 | 6.9 |
| PD240214SFM | A | 1 | 7 | 16.2 | NA |
| | | 2 | 14 | 20.8 | 2.7 |
| | | 3 | 20 | 19.4 | 6.4 |
| | B | 1 | 7 | 22 | NA |
| | | 2 | 14 | 18.2 | 2.1 |
| | | 3 | 20 | 19.2 | 6.1 |
| PD230414SFM | NA | 1 | 7 | NA | NA |
| | | 2 | 14 | NA | 2.3 |
| | | 3 | 19 | 16.2 | 5.7 |
| PD040514SFM | NA | 1 | 7 | NA | NA |
| | | 2 | 14 | NA | 2.7 |
| | | 3 | 18 | 15.6 | 6.5 |
| PD260514SFM | NA | 1 | 7 | NA | NA |
| | | 2 | 13 | NA | 2.9 |
| | | 3 | 17 | 15.8 | 6.6 |
| PD180814SFM | NA | 1 | 6 | NA | NA |
| | | 2 | 10 | NA | 2.1 |
| | | 3 | 16 | 16.7 | 5.3 |
| PD220914SFM | unfiltered | 1 | 8 | NA | NA |
| | | 2 | 14 | NA | 2.1 |
| | | 3 | 20 | 17 | 5.6 |
| | filtered | 1 | 8 | NA | NA |
| | | 2 | 14 | NA | 2 |
| | | 3 | 20 | 17.8 | 5.1 |
| PD271014SFM | filtered | 1 | 9 | NA | NA |
| | | 2 | 15 | NA | 2.1 |
| | | 3 | 21 | 17 | 5.1 |

| | | | |
|---|---|---|---|
| Average P 3 | 19.1 | 17.55 | 6.12 |
| %CV P 3 | 8 | 9 | 11 |

### Example 3: Additional Culturing Steps

### Step 2-1: Additional Two-Dimensional (2D) Cell Culturing.

The ICS was thawed, diluted with and cultured in StemPro® medium until 60-90% confluence (typically 4-7 days after seeding), and cultured for 2 additional passages (referred to as passages 3/1 and 3/2 respectively; again passaging when reaching 60-90% confluence), then were harvested for seeding in the bioreactor.

### Step 2-2: Three Dimensional (3D) Cell Growth in Bioreactor/s

Each bioreactor contained Fibra-cel® carriers (New Brunswick Scientific) made of polyester and polypropylene, and StemPro® medium.

The culture medium in the bioreactor/s was kept at the following conditions: temp: 37±1° C., Dissolved Oxygen (DO): 70±20% and pH 7.4±0.4. Filtered gases (Air, CO₂, N₂ and O₂) were supplied as determined by the control system in order to maintain the target DO and pH values.

After seeding, the medium was stirred with stepwise increases in the speed, up to 150-200 RPM by 24 hours. Perfusion was initiated several hours after seeding and was adjusted on a daily basis in order to keep the glucose concentration constant at approximately 550 mg\liter.

Cells were typically harvested after 5-6 days by washing the cells, adding trypsin, and subjecting them to agitation.

### Step 2-3: Downstream Steps: Concentration, Washing, Formulation, and Cryopreservation

Cells were suspended and washed in suspension solution (5% w/v human serum albumin [HSA] in isotonic solution), then adjusted to 10-20×10⁶ cells/ml, in isotonic solution with 10% DMSO v/v and 5% HSA w/v. The vials were gradually chilled and stored in a gas-phase liquid nitrogen freezer.

### Example 4: Production of ASC with Different HLA Types and Similar Therapeutic Characteristics

Methods

### Endothelial Cell Proliferation (ECP)

1 million ASC were seeded in 2 mL DMEM medium, in a 6-well plate in triplicate. After 20 hours, the medium was replaced with EBM-2 medium, and cells were incubated under hypoxic conditions (1% O₂) for an additional 24 hours. Afterwards, the conditioned media (CM) was collected. In parallel, 750 Human Umbilical Vein Endothelial Cells (HUVECs) cells per well of 96-well plate were seeded and incubated for 24 hours in EBM-2, and then incubated with the ASC-CM, for 4 days under normoxic conditions (21% O₂) at 37° C. After removal of the CM, the proliferation of the HUVEC cells was assayed using the alamarBlue® fluorescent assay. Results are presented as percent of ECP, relative to percent proliferation of a reference batch known to elicit robust ECP activity.

### Monocyte IL-10 Secretion Assay

ASC were resuspended in X-VIVO™ 15 medium+10% FBS (Lonza), and 3000 ASC/well were seeded in 150 microliters (mcL) in 48-well plates. After 24 hours, 2×10⁴ U937 cells in 100 mcL were added, and plates were incubated for an additional 17 hours. LPS was added, the cells were incubated for 5 more hours, and IL-10 in the supernatant was measured by ELISA and compared to a reference batch known to elicit robust IL-10 production.

### Luminex® Assays, RayBiotech Cytokine Array, and ELISAs

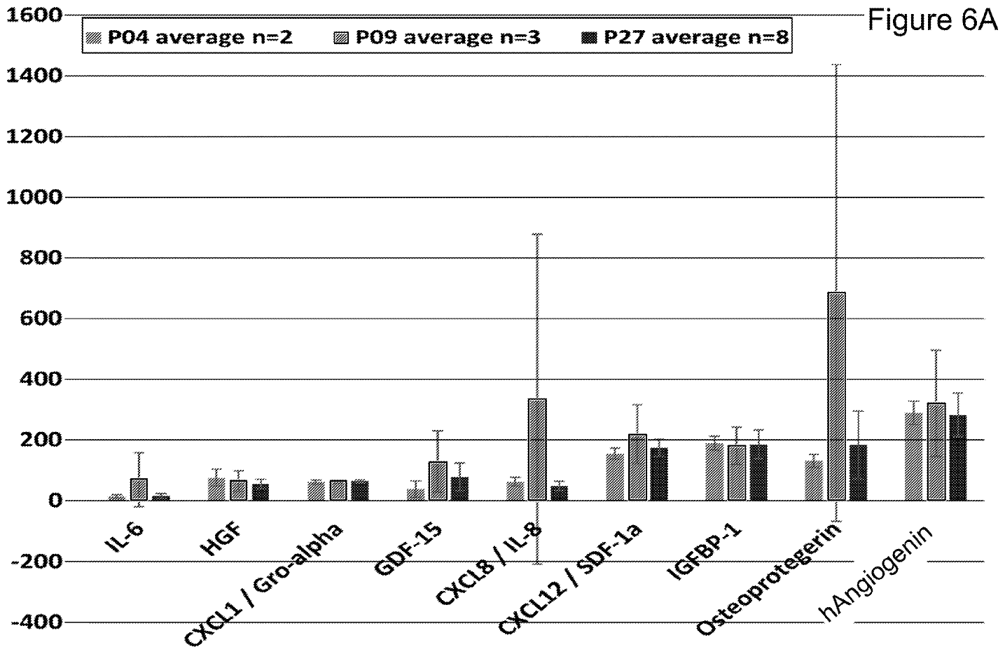

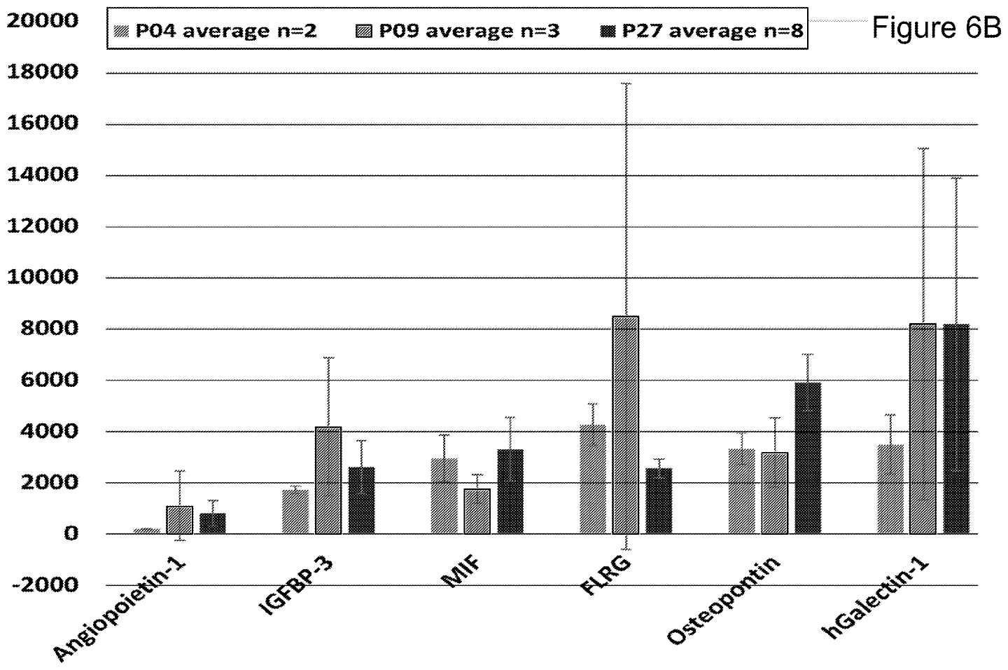

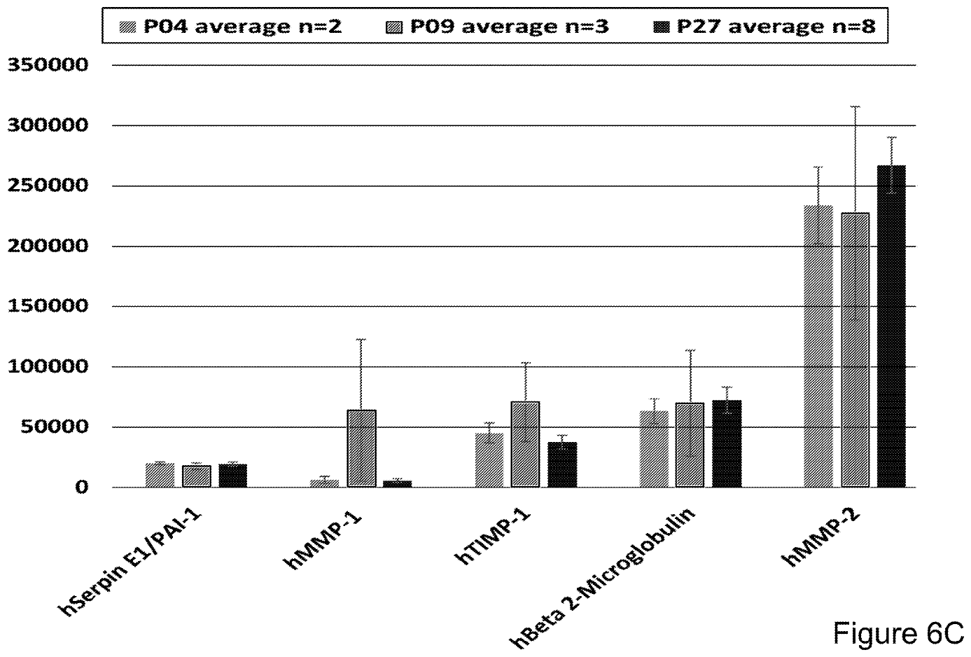

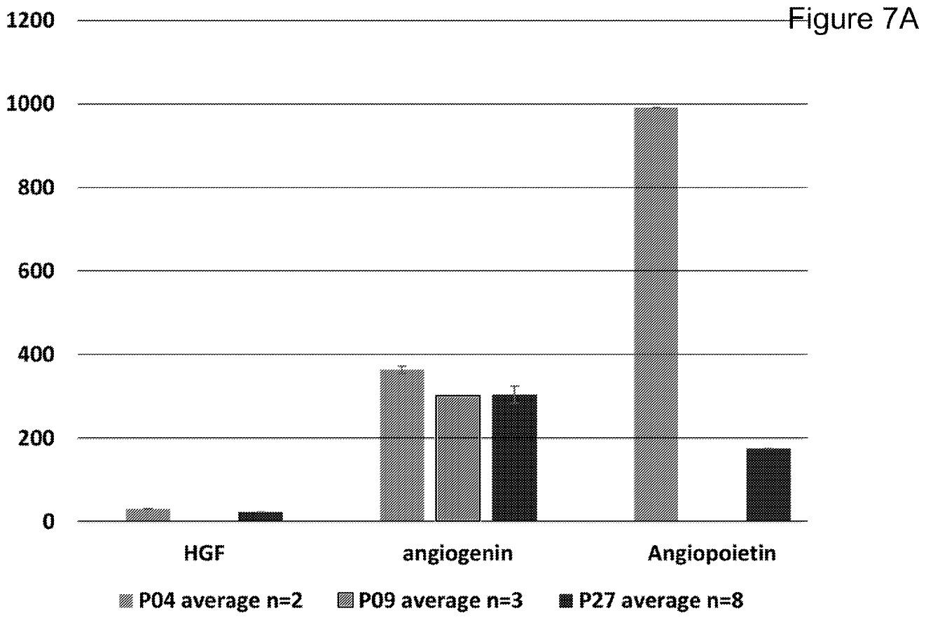

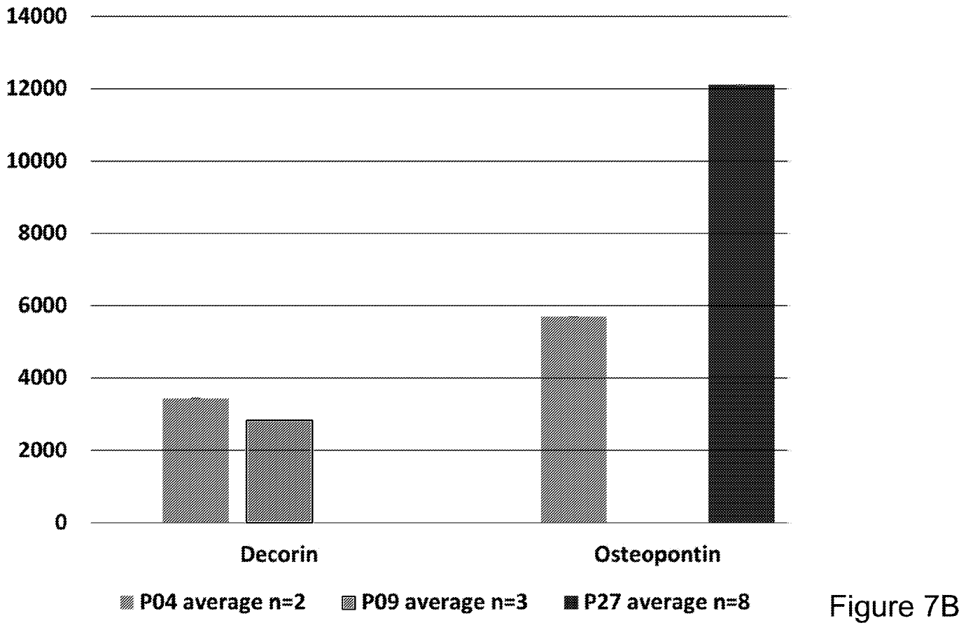

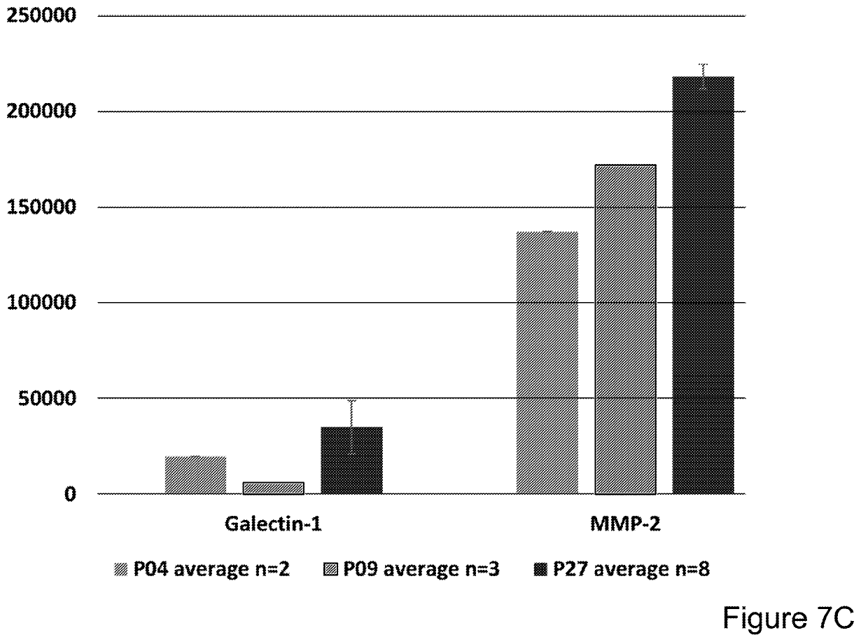

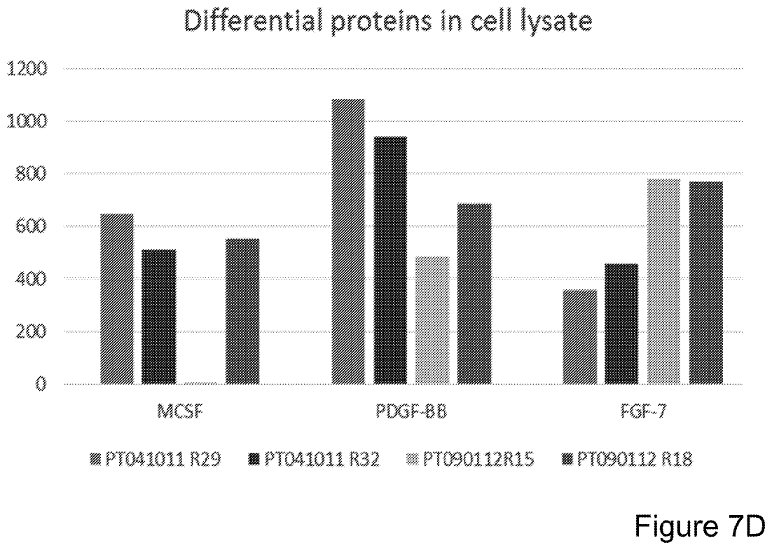

CM was prepared from ASC as described for the ECP assay. For each ICS/source placenta (04, 09, and 27, as described below), several bioreactor runs were obtained, tested in parallel, and used to generate a mean and standard error. Luminex® assays (FIG. 6) were performed as per the extracellular assay protocol provided by the manufacturer. Assay standards enabled conversion of raw data to cytokine concentrations in pg./ml. ELISA (FIGS. 7A-C) were also performed on the CM, using kits from R&D Systems (Minneapolis, Minn.), except for HGF and Decorin, which were from RayBiotech (Norcross, Ga.) and Abcam (Cambridge, Mass.), respectively. Protein levels in the CM are expressed as pg./ml. RayBiotech cytokine arrays (FIG. 7D) were used, as per the manufacturer's instructions, but using 0.5×10⁶ cells in 1 milliliter.

### PBMC Proliferation Inhibition Assay

ASC cells were seeded at 0.1×10⁶ cells/well) in 24 well plates, in 1 ml of RPMI 1640 medium, supplemented with 10% FBS, 2 mM L-Glutamine and 50 mcg/ml Gentamycin. After 24 h incubation in 37° C., the medium was removed from the wells, and CFSE ((5(6)-Carboxyfluorescein N-hydroxysuccinimidyl ester)-labelled PBMCs (0.5×10⁶ cells/well) from two different donors were seeded in the wells (one plate for each donor), at a 1:5 ratio of ASC: PBMCs. PBMCs were also seeded without ASC and served as a control. The cells in all wells were stimulated with 15 mg/ml PHA and incubated for 5 days in 37° C. Proliferation was analyzed by flow cytometry on gated cells, defined as lymphocytes according to their size and granularity. For each ICS, 4 bioreactor runs were tested in parallel and tested with both PBMC donors.

Results

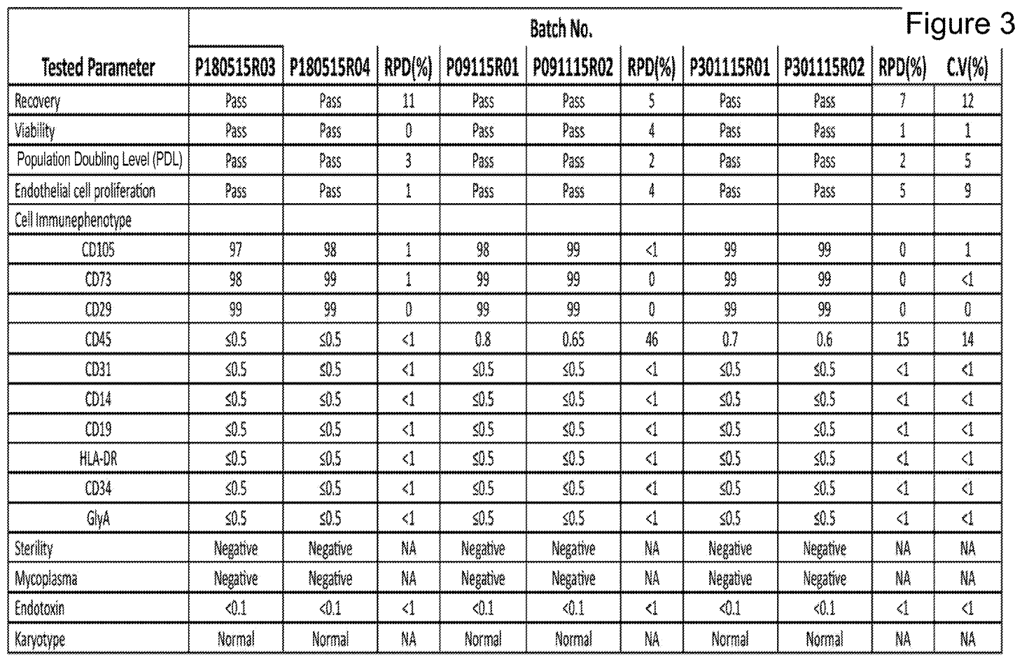

ASC were prepared from various donor placentas and subjected to 2D, followed by 3D culture, as described in Example 1. The cells removed from the 3D carriers exhibited a high degree of consistency in various characteristics, including immuno-phenotype, karyotype, population doubling level (PDL) and ECP activity (FIG. 3), and similar GCR (Table 4), while having different HLA types.

TABLE 4
GCR of different placental ASC batches in mg/day.

| Batch | Parameter | Day 3 | Day 4 | Day 5 | Day 6 |
|---|---|---|---|---|---|
| 04 | Average | 2149 | 4444 | 7624 | 11593 |
| | SE | 83 | 211 | 702 | 318 |
| 09 | Average | 3024 | 5888 | 9927 | 13953 |
| | SE | 57 | 145 | 244 | 377 |
| 27 | Average | 2058 | 4146 | 7043 | 10464 |
| | SE | 52 | 94 | 176 | 182 |

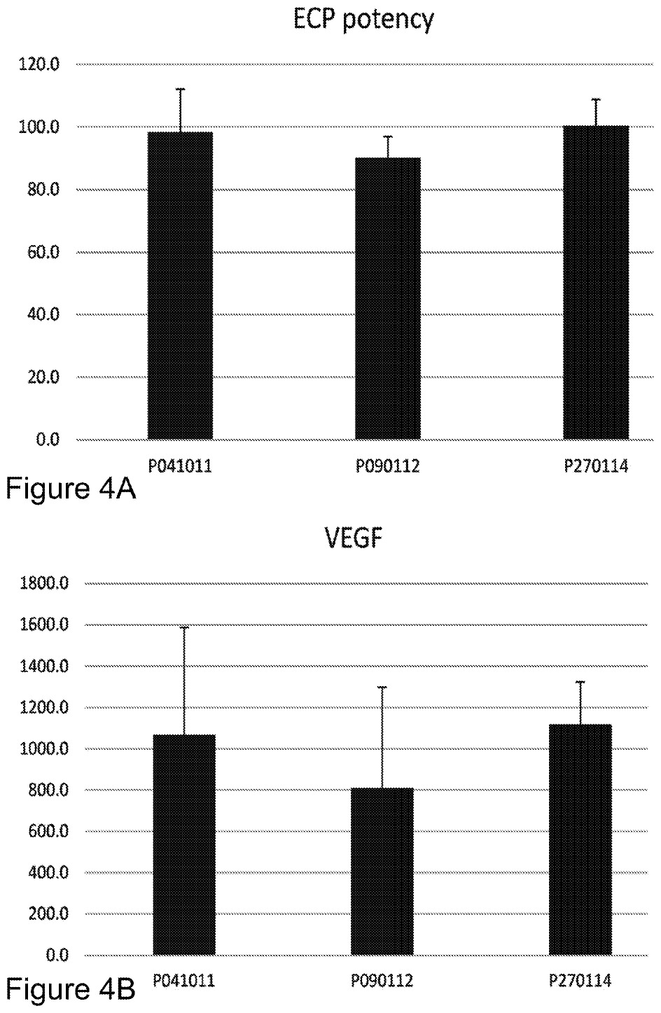

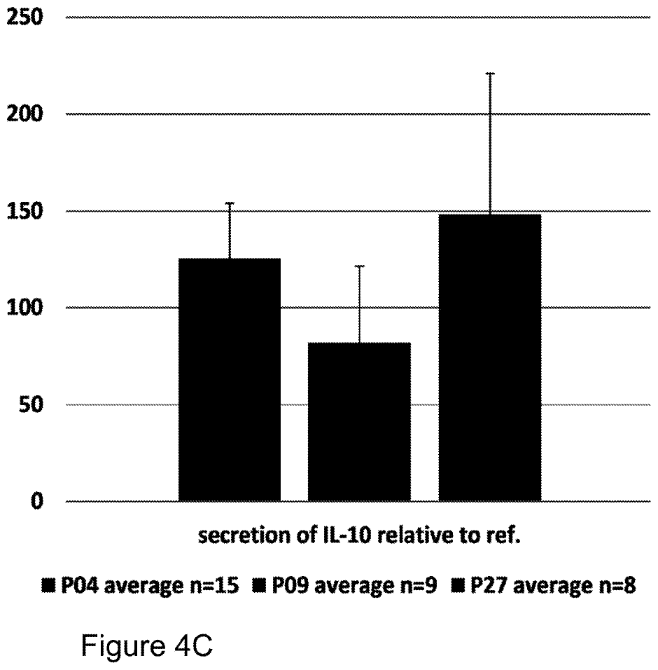

ASC from various placentas (each stored as an ICS with a unique identifier) also exhibited similar activity after 2D+3D in the ECP assay and in their VEGF secretion, as shown for 3 representative batches, P041011 (“04”), P090112 (“09”), and P270114 (“27”) (FIGS. 4A-B). In other experiments, ASC from the different placentas were incubated with U937 cells (monocytes), and IL-10 secretion from the monocytes was measured by ELISA. The different ASC populations elicited similar amounts of IL-10 secretion (FIG. 4C).

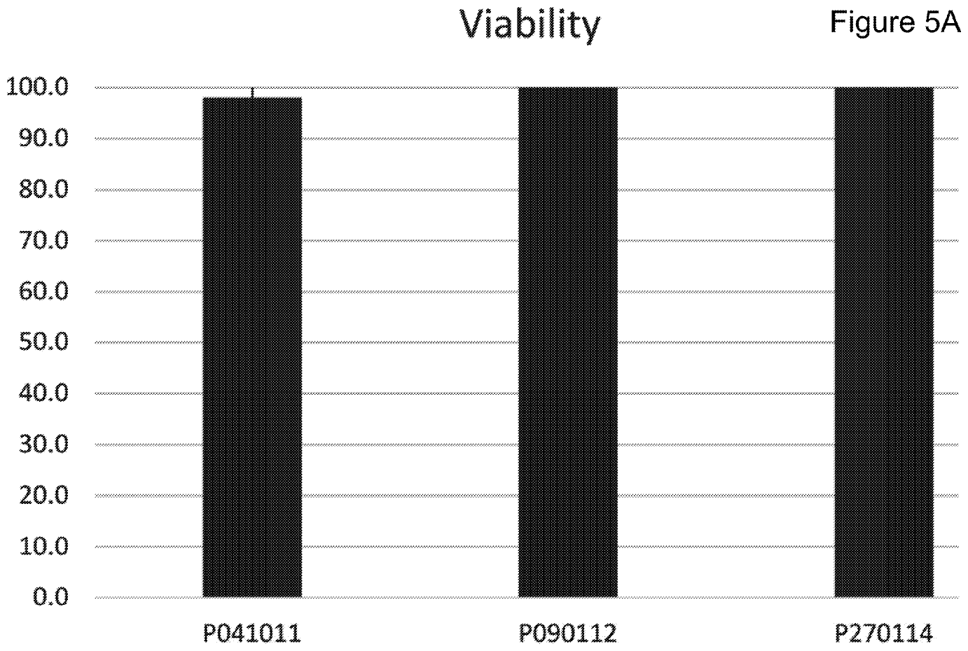

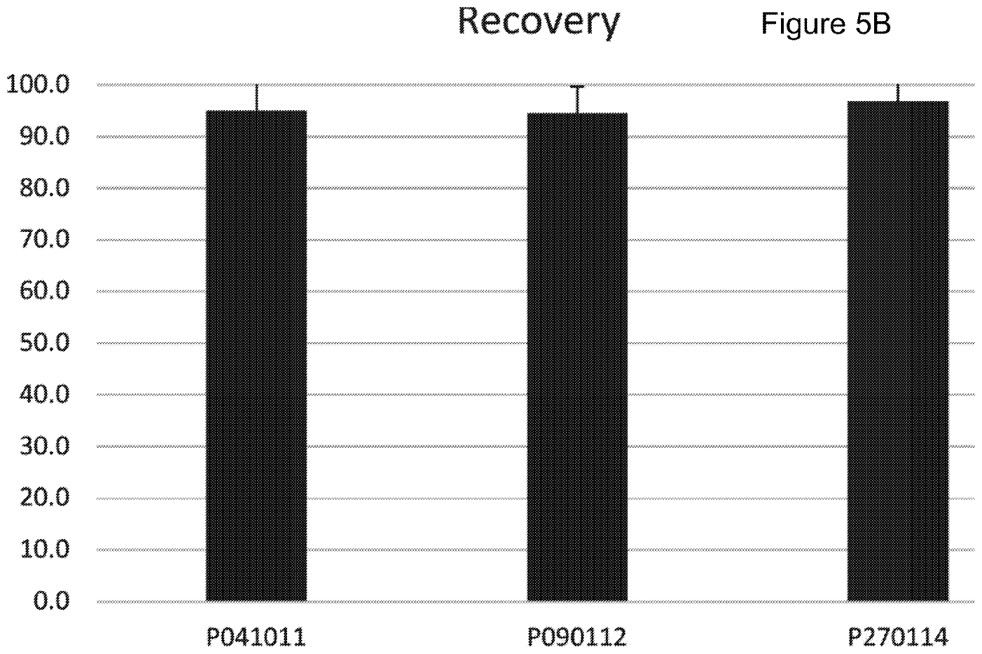

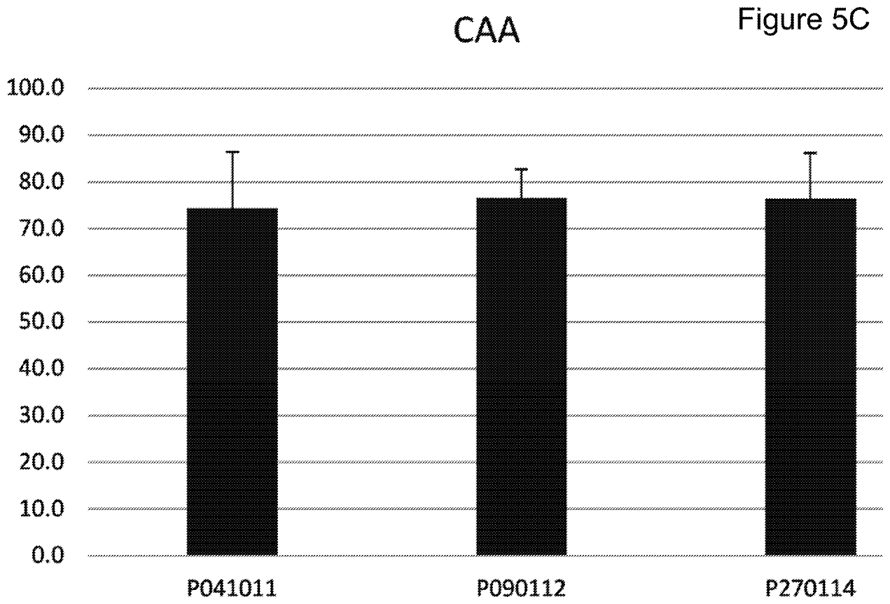

The 04, 09, and 27 batches exhibited a high degree of consistency in parameters in their percent viability, percent recovery and cell adhesion assay (FIGS. 5A-C). Levels of secreted VEGF were 800-1100 pg./ml×2 ml=1600-2200 pg. total, which can also be expressed as 1600-2200 pg. per million cells seeded.

Secretion of various cytokines by ASC from the aforementioned placentas was measured by Luminex® assays (FIG. 6) and ELISA (FIGS. 7A-C). Specifically, Luminex® was used to measure levels of IL-6, HGF, Gro-alpha (GROa), IL-8, SDF-1 alpha, IGFBP-1, Osteoprotegerin, and Angiogenin (6A); Angiopoietin-1, IGFBP-3, MIF, FLRG, Osteopontin, and Galectin-1 (6B); and Serpin E1, MMP-1, TIMP1, Beta2 microglobulin, and MMP-2 (6C). ELISA was used to measure HGF, Angiogenin, and Angiopoietin-1 (7A); Decorin and Osteopontin, and (7B); and Galectin-1 and MMP-2 (7C). The different ASC populations secreted similar amounts of the tested cytokines; the highest numbers were often not more than 2-fold the lowest numbers.

Additionally, 2 batches each from the 04 and 09 placentas were tested for secretion of M-CSF, PDGF-BB, and FGF-7 (FIG. 7D). For each cytokine, the highest numbers were typically not more than 2-fold the lowest numbers.

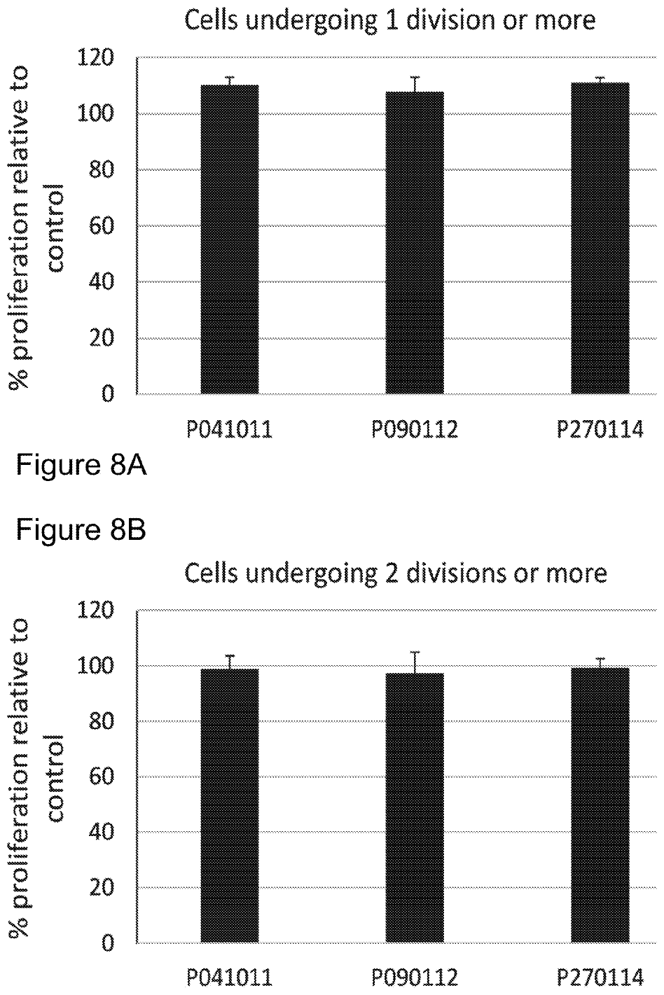

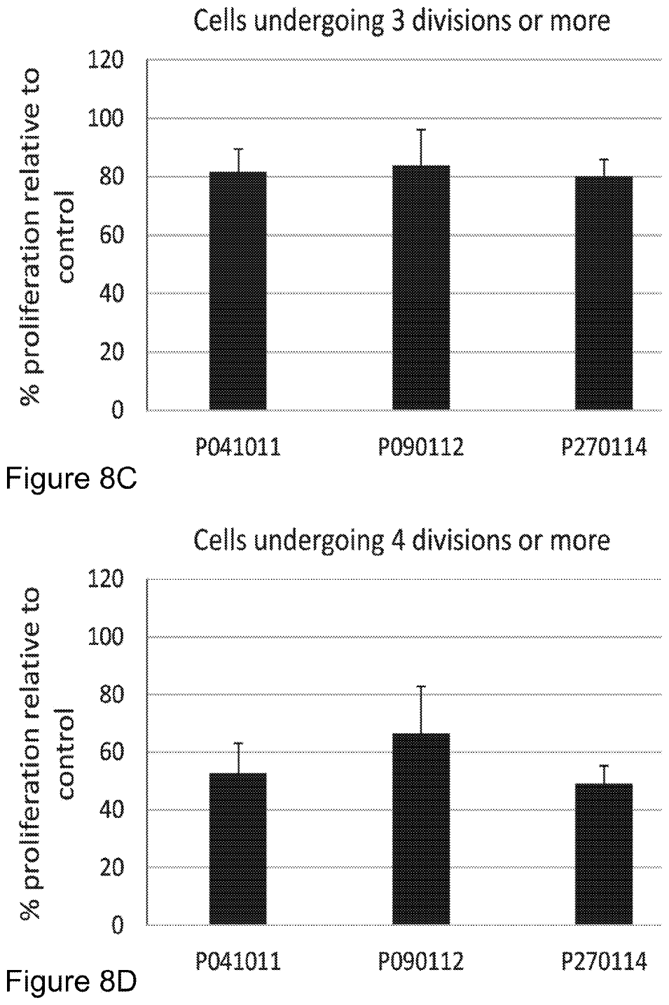

ASC from different placentas also exhibited similar activity in PMBC proliferation assays, each inhibiting PMBC proliferation to a comparable extent (FIGS. 8A-D).

In conclusion, the described methods enable production of cell populations that have different HLA types, while exhibiting a high degree of consistency in various indicators of quality and therapeutic efficacy. Similar results were obtained when ASC were prepared from different donor placentas and cultured as described in Examples 2-3.

The 04, 09, and 27 batches were used to generate the clinical data in the next Example.

### Example 5: Serial Administration of ASC with Different HLA Types Confers Superior Therapeutic Efficacy

### Overview:

A Phase II, multicenter, multinational, randomized, double-blind, placebo-controlled, parallel-groups study was performed, to test the safety and efficacy of placental ASC in patients with intermittent claudication (IC) due to peripheral arterial disease (PAD). The study contained 4 treatment groups:

Group #1: (“low dose”): First and second treatment were each 150×10⁶ placental ASC. Group #2: (“high dose”): First and second treatment were each 300×10⁶ placental ASC. Group #3: (“placebo”): First and second treatment were each placebo (15 mL Vehicle). Group #4: (“single treatment high dose+single treatment placebo”): First treatment was 300×10⁶ placental ASC; second treatment was placebo (15 mL Vehicle).

Approximately 170 subjects aged 45 to 85 years and diagnosed with IC due to PAD were enrolled, with 37, 48, 50, and 37 patients treated in Groups 1-4, respectively. 33, 42, 45, and 33 patients, respectively, were included in the mFAS (described below).

Subjects received the indicated treatments in the affected leg, with a 12-week interval between treatments.

The study contained 6 stages:
1. Screening Period of up to 4 weeks.
2. First treatment of placental ASC or placebo at week 0.
3. Short-term follow-up at 24 hours after first treatment, and weeks 1 and 4 after first treatment.
4. Repeat dose of placental ASC or placebo at week 12 after first treatment.
5. Short-term follow-up 24 hours after second treatment.
6. Long-term follow-up at weeks 13, 26, 39 and 52 after first treatment. Study termination at week 65 after first treatment.

### Detailed Methodology:

### Stage I, Screening Period: Week-4-0

The screening period included screening number assignment and diagnosis confirmation.

Inclusion/Exclusion assessment, including two baseline Exercise Treadmill Tests (ETTs) performed with a time interval of 7-10 days, demographic information, and medical history, including an allergy history questionnaire and concomitant medication. A washout period of at least 2 weeks was observed from vasodilators prescribed for IC prior to the first ETT. Vital signs, physical examination, ECG, ABI and/or TBI, and laboratory tests (serum pregnancy test, hematology, blood chemistry, urinalysis and coagulation profile) were collected and analyzed.

After eligibility was confirmed, subjects were randomized to receive 1 of 2 target doses of placental ASC or placebo at least 1 week prior to the planned first treatment.

### Stage II, Visit 1a (First Dose Treatment): Week 0

On the first treatment day and prior to the placental ASC/placebo treatment, the following were performed: Inclusion/Exclusion re-assessment, vital signs, ECG, resting ABI and/or TBI, laboratory tests (urine pregnancy test [for women of child-bearing potential], urinalysis, hematology, blood chemistry, IL-6 testing for a subset of the subjects (immunology profile), HLA-typing, HLA-Abs and tryptase levels were collected. Two health-related Quality of Life Questionnaires (QoL SF-36v2 and Peripheral Arterial Questionnaire (PAQ)) were administered. AEs and concomitant medications were recorded.

Antihistamine pre-treatment was given 1 hour (±15 minutes) prior to the study treatment administration to ensure coverage for 24 hours and as long as necessary post study treatment administration.

Pre-medication with analgesics was administered to the subject at the Investigator's discretion as long, as it did not require cardio-respiratory monitoring of the subject.

Upon satisfactory completion of the pre-treatment steps, placental ASC/placebo was administered after 30 minutes of rest via 30 intramuscular injections delivered to the most affected leg. The most affected leg was defined as the leg with the lowest ABI and/or TBI at screening. However, in cases where the leg with the lowest ABI and/or TBI at screening was not the most symptomatic leg (i.e. the leg that limits the subject's walking), then the investigator injected the most symptomatic leg according to his clinical judgment, as long as it fulfilled the ABI and/or TBI inclusion criteria. After a 1-hour monitoring period, vital signs were measured and AEs were recorded.

If an allergic/hypersensitivity reaction occurred while the subject was still under medical assessments, blood samples for tryptase values were collected immediately. For subjects developing an allergic/hypersensitivity reaction following discharge, additional tryptase blood samples were collected within 4 hours of the first appearance of the allergic/hypersensitivity reaction, or as soon as possible thereafter.

### Stage III, Visits 1b 2 and 3 (Short Term Follow Up): 24 Hours After First Dose Treatment

### Weeks 1 and 4

Visit 1b: 24 hours after initial treatment, AEs, vital signs (including pulse oximetry measurement), and physical examination were performed, and blood samples for tryptase levels were collected.

Visit 2: week 1 after initial treatment, vital signs, ECG, AEs, concomitant medication and laboratory blood tests [(hematology, blood chemistry, IL-6 measurement, and HLA-Abs] were collected and analyzed.

Visit 3: week 4 after initial treatment, vital signs, AEs, concomitant medication and laboratory blood tests (hematology, blood chemistry) were collected and analyzed.

### Stage IV, Visit 4a (Repeat Dose Treatment): Week 12

On the day before the second dosing, vital signs, ECG, resting ABI and/or TBI, health-related QoL Questionnaires, ETT, AEs, concomitant medication, and laboratory tests (urine pregnancy test, urinalysis, hematology, blood chemistry, and IL-6 measurement) and HLA-Abs and tryptase levels were collected and analyzed.

If any subjects developed a severe allergic/hypersensitivity reaction that required hospitalization and/or treatment with intravenous steroids/epinephrine following visit 1a, or for whom, in the opinion of the investigator, the risk of developing such severe allergic/hypersensitivity reactions increased since the screening, they were contraindicated from receiving the second dosing.

The second dosage was given in the same affected leg treated at visit 1a. The dosage and immediate follow-up protocols were essentially identical to the first dosing.

### Stage V, Visit 4b (Short Term Follow Up): 24 Hours After Repeated Dose Treatment

Visit 4b, 24 hours after second treatment: AEs, vital signs (including pulse oximetry measurement), physical examination and blood samples for tryptase levels were collected and analyzed.

### Stage VI (Long Term Follow Up): Weeks 13, 26, 39 and 52, Termination Visit (Week 65) and Unscheduled Visit

During the long term follow up period, subjects visited the Medical Center on weeks 13, 26, 39, 52 and 65 (termination visit) for follow-up by a clinical Investigator.

Visit 5, week 13: One week (±1 day) after the second treatment, vital signs, ECG, AEs, concomitant medication and laboratory tests (hematology, chemistry, IL-6 measurement) and HLA-Abs were collected and analyzed.

Visit 6, week 26: vital signs, resting ABI and/or TBI, 2 health-related QoL Questionnaires, ETT, AEs and concomitant medication were recorded.

Visit 7, week 39: vital signs, resting ABI and/or TBI, 2 health-related QoL Questionnaires, ETT, AEs and concomitant medication and laboratory tests (hematology and chemistry) were collected and analyzed.

Visit 8, week 52: vital signs, resting ABI and/or TBI, 2 health-related QoL Questionnaires, ETT, AEs and concomitant medication and laboratory tests (hematology and chemistry) were collected and analyzed.

Termination Visit, week 65/early discontinuation: vital signs, physical examination, ECG, resting ABI and/or TBI, 2 health-related QoL Questionnaires, ETT, AEs, concomitant medication, and laboratory tests (urine pregnancy test [women of child-bearing potential], urinalysis, hematology, HLA-Abs and blood chemistry) were collected and analyzed.

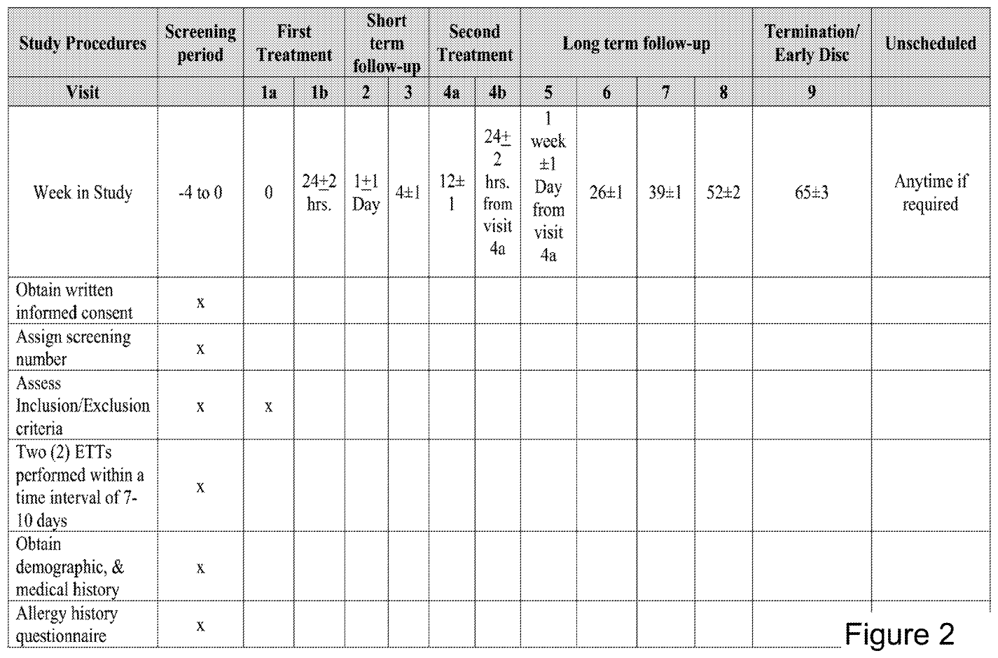

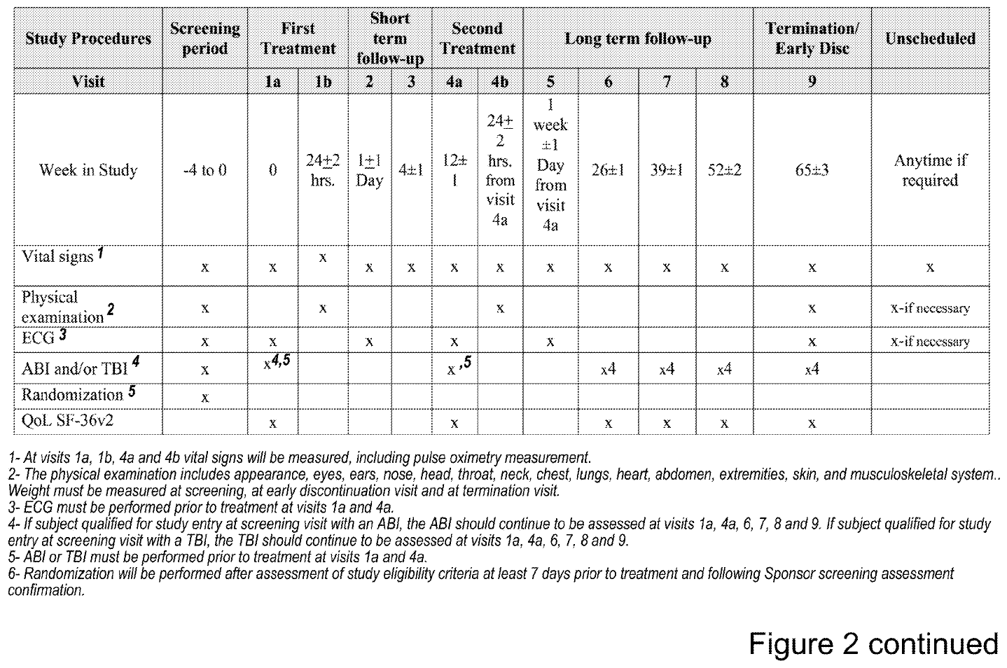

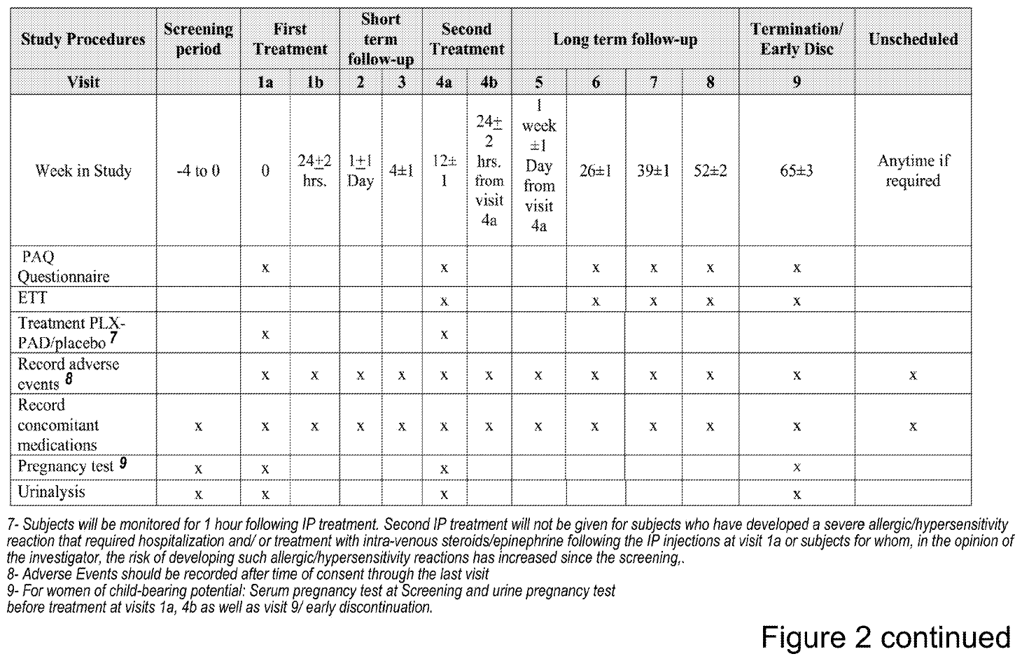

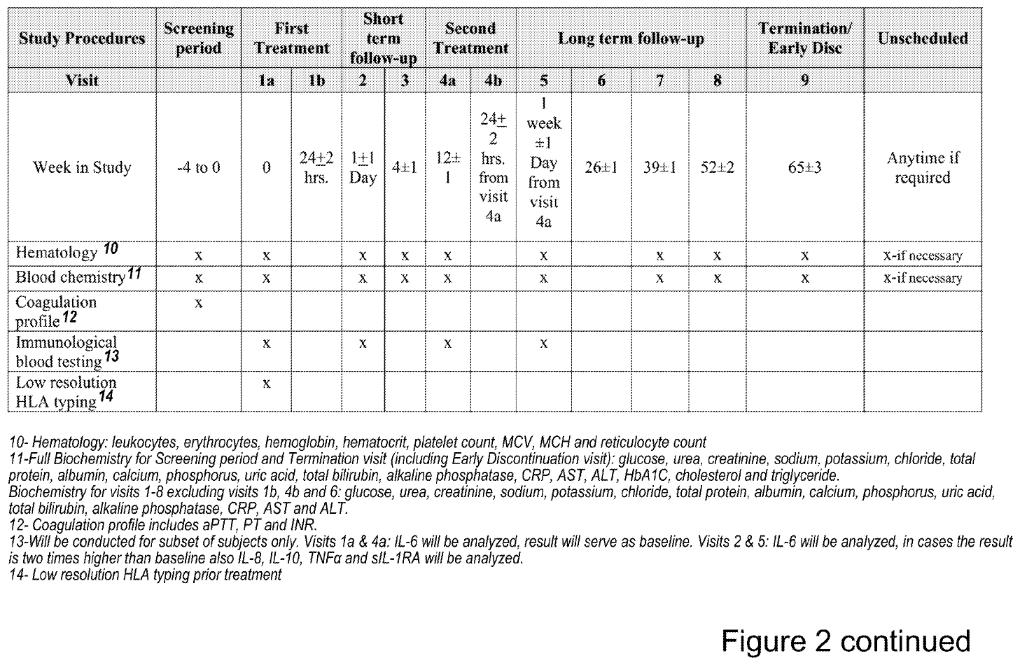

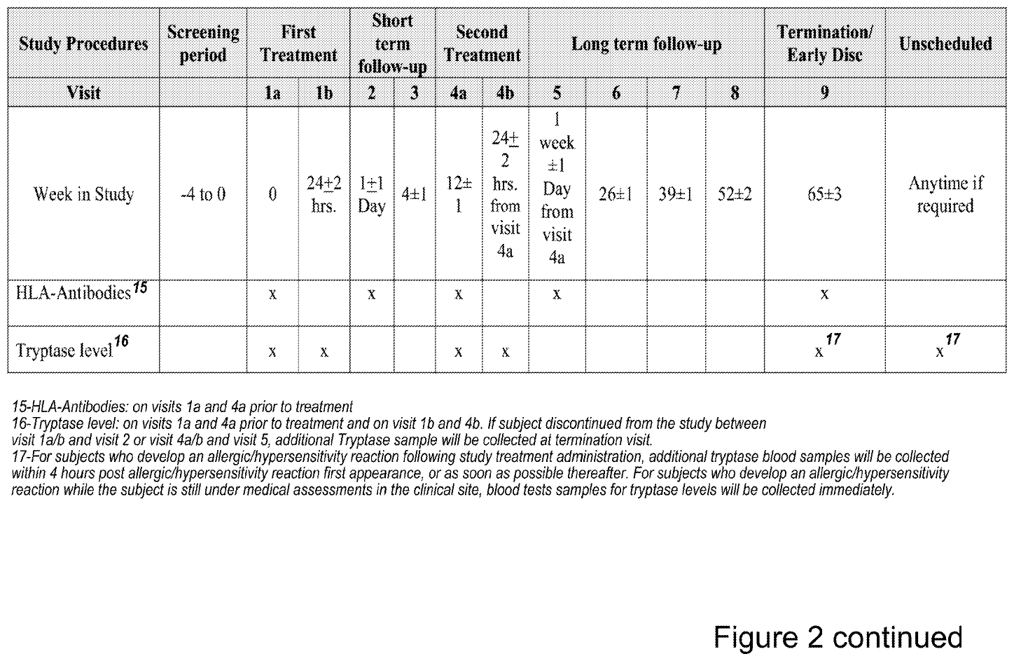

The study visit flow chart is depicted in FIG. 2.

For immunological profile, blood samples were tested for levels of IL-6, IL-8, IL-10, TNF-α, and sIL-1RA where applicable.

### Safety Endpoints:

Treatment emergent adverse events, SAEs, AEs leading to premature study termination.
Safety laboratory values
Immunological reaction
Major Amputation of the Lower Extremity
Death rates

### Efficacy Endpoints:

### Primary Endpoint:

Log ratio of week 52 MWD to baseline MWD

### Secondary Endpoints:

Log ratio of week 52 MWD to baseline MWD patients that received cell from 2 different donors
Log ratio of week 52 ICD to baseline ICD.
Change from baseline to Week 52 in Peripheral Arterial Questionnaire (PAQ)
Change from baseline to Week 52 in Quality of Life (QoL) Questionnaire (SF-36v2)
Change—baseline to Week 52 in hemodynamic measurements (resting ABI and/or TBI)
Revascularization rates at week 52.

### Study Population

This study was conducted in subjects aged 45-85 years and diagnosed with IC due to PAD.

### Inclusion Criteria:

Subjects needed to meet all of the inclusion criteria listed below to be eligible for the study:
1. Age between 45 to 85 years of age (inclusive) at the time of screening visit.
2. Subjects with a diagnosis of peripheral artery disease, secondary to atherosclerosis, confirmed by one of the following criteria assessed at the screening visit:
   Resting ankle-brachial index (ABI) ≤0.80; or
   Resting ABI<0.90 and >20% decrease in ABI from rest to exercise when measured within 1 minute after treadmill exercise; or
   Toe-brachial index (TBI) <0.60 (if ABI>1.3, TBI should be assessed)
3. Lifestyle-limiting, moderate to severe claudication (symptoms present and stable for >6 months and not significantly changed within the past 3 months prior to screening).
4. Evidence of significant (>50%) stenosis infra-inguinal occlusive disease (distal to the common femoral artery) as confirmed by documented results from Duplex, MRA, CTA and/or contrast angiogram completed within 3 months prior to screening.
5. The longest maximal walking distance (MWD) from the Screening Period exercise treadmill tests (ETT), utilizing a modified Gardner Protocol (Table 5), must be between 1 and 10 minutes (inclusive).

TABLE 5
Modified Gardner Protocol.

| | Speed | Elevation | Time |
|---|---|---|---|
| Stage | (mph) | (% grade) | (min) |
| Rest* | 2.0 | 0 | — |
| 1 | 2.0 | 0 | 2 |
| 2 | 2.0 | 2 | 2 |
| 3 | 2.0 | 4 | 2 |
| 4 | 2.0 | 6 | 2 |
| 5 | 2.0 | 8 | 2 |
| 6 | 2.0 | 10 | 2 |
| 7 | 2.0 | 12 | 2 |
| 8 | 2.0 | 14 | 2 |
| 9 | 2.0 | 16 | 2 |
| 10 | 2.0 | 18 | as needed to |
| | | | reach MWD |
| Recovery** | 0.0 | 0 | N/A |

6. Subjects who have persistent claudication symptoms despite having been recommended an exercise program if feasible, and/or despite having been on a stable dose of a vasodilator prescribed for IC (including Cilostazol, Pentoxifylline, Naftidrofuryl, and prostanoids), if indicated. Subjects that were previously receiving a vasodilator prescribed for IC were washed out for at least 2 weeks prior to the first ETT.
7. Subjects should be receiving standard of care drugs for vascular disease, including antiplatelet agent(s) and statin medication, as well as anti-hypertensive medication(s) and oral hypoglycemic agents/insulin, if indicated./insulin, if indicated.
8. Signed written informed consent.

### Exclusion Criteria:

Subjects with any one of the exclusion criteria listed below were not eligible for the study:
1. Ischemic rest pain; ulceration or gangrene (Fontaine class III-IV; Rutherford category 4-6).
2. Failed lower extremity arterial reconstruction (surgical or endovascular) or sympathectomy within the prior one month of screening.
3. Planned revascularization (surgical or endovascular intervention) within 12 mo. after screening.
4. Lower extremity arteries inflow obstruction (defined as a greater than 50% stenosis of aorta, iliac and/or common femoral arteries).
5. History of Buerger's disease.
6. Uncontrolled hypertension (defined as diastolic blood pressure >100 mmHg or systolic blood pressure >180 mmHg during screening).
7. Uncontrolled diabetes (defined as HbA1c >9% at screening).
8. Life-threatening ventricular arrhythmia—except in subjects with an implantable cardiac-defibrillator.
9. Serum Creatinine level >2.5 mg/dl.
10. SGPT (ALT), SGOT (AST) >2.5× upper limit of normal range.
11. Hemoglobin <10 g/dl.
12. Unstable cardiovascular disease defined as myocardial infarction (STEMI or NSTEMI) within 3 months prior to screening, or unstable angina—characterized by increasingly frequent episodes with modest exertion or at rest, worsening severity, and prolonged episodes.
13. Transient Ischemic Attack (TIA)/Stroke within 3 months prior to screening.
14. Subjects with severe congestive heart failure symptoms (i.e. NYHA Stage III to IV).
15. Subjects with implant of mechanical prosthetic heart valve(s).
16. Pulmonary disease requiring supplemental oxygen treatment on a daily basis.
17. Active significant infection including but not limited to osteomyelitis, fasciitis, or severe/purulent cellulitis.
18. History of malignancy within 5 years prior screening, including basal cell carcinoma (BCC) or squamous cell carcinoma (SCC) of the treated leg (a subject with BCC or SCC of the opposite leg can be included).
19. Walking limited by any condition other than PAD, including but not limited to spinal stenosis, congestive heart failure, chronic pulmonary disease, angina pectoris, or degenerative joint disease.
20. Subjects who are on oral anticoagulant therapy. Unless, upon the primary care physician and/or investigator's discretion, the treatment can be safely interrupted/discontinued around each IP injection to reduce risk of hemorrhage.
21. Immunocompromised subjects for any reason at screening.
22. Known allergies to any of the following: dimethyl sulfoxide (DMSO), human serum albumin, bovine serum, or recombinant trypsin.
23. Known sensitivity to Gentamycin.
24. Known sensitivity to antihistamine drugs.
25. History of allergic/hypersensitivity reaction to any substance having required hospitalization and/or treatment with intra-venous steroid/epinephrine, or in the opinion of the investigator the subject is at high risk of developing severe allergic/hypersensitivity reactions.
26. History of acute transfusion reaction or history of autologous/allogeneic bone marrow or solid organ transplantation.
27. History of uncontrolled Asthma (GINA III-IV) or Chronic Urticaria.
28. Medical history of Human Immunodeficiency Virus (HIV) or syphilis positivity at time of screening.
29. Known active Hepatitis B, or Hepatitis C infection at the time of screening.
30. Pregnant or breast-feeding women or women of childbearing age not protected by an effective contraceptive method of birth control (such as double barrier, oral or parenteral hormonal, intrauterine device and spermicide).
31. In the opinion of the Investigator, the subject is unsuitable for participating in the study.
32. Subject is currently enrolled in, or has not yet completed a period of at least 30 days since ending other investigational device or drug trial(s).
33. Subjects that have prior exposure to gene or cell-based therapy.

### Ankle-Brachial Index (ABI)/Toe-Brachial Index (TBI)

The ABI for each leg was defined as the ratio between the higher of the two pedal systolic blood pressure measurements (dorsalis pedis and posterior tibialis) normalized to the higher of the two systolic brachial pressure measurements (right or left). A continuous wave Doppler ultrasonic instrument, with an operating frequency between 5 and 10 megahertz (MHz), was used to measure the systolic pressures in both the dorsalis pedis and posterior tibialis arteries in each leg, as well as the brachial arteries in each arm. The higher of the 2 arm pressures and the higher of the 2 ankle pressures for each leg were used for the calculation. The subject rested supine for at least 10 minutes prior to obtaining pressure measurements used to calculate resting ABI. ABIs were performed by trained study personnel according to the schedule of assessments.

A false-normal or false high ABI due to arterial media calcification is common in diabetics (about 30%). Therefore, inclusion criterion “low ABI” may be misleading or false in a considerable number of potential study participants. The diagnosis of media calcification and therefore non-reliable ABI was made when the ABI was higher than 1.30 for either leg. If the subject had a diagnosis of media calcification with an ABI greater than 1.30 for either leg, a TBI was obtained instead.

The examination of the TBI was similar to the ABI except that it was performed with a photoplethysmograph (PPG) infrared light sensor and a very small blood pressure cuff placed around the toe, and is a calculation based on the systolic blood pressures of the arm and the systolic blood pressures of the toes. If a photoplethysmograph (PPG) was not available, TBI was performed using a continuous wave ultrasonic Doppler with an operating frequency between 5-10 megahertz.

### Exercise Treadmill Tests

The primary endpoint of MWD was assessed using a graded treadmill protocol. This test is reproducible in assessing peak and submaximal exercise performance in patients with PAD and claudication (Hiatt W R, 1988; Gardner A W, 1991). Gardner determined that the graded test was more stable than a constant-load test. Subsequent evaluation of the published literature comparing the graded test with the constant-load test determined that the graded test had the highest reliability when using the ACD as the primary endpoint (Nicolai S P, 2009). Treadmill Familiarization was performed during the Screening visit and as deemed necessary during subsequent visits (e.g., prior to an ETT) to acquaint subjects with the treadmill equipment and requirements of the ETT. Brief periods of walking were conducted at a slow speed of 1.0 mile per hour (mph) followed by increasing increments of 1.5 mph to the speed of 2.0 mph, all at 0% grade. Whenever Treadmill Familiarization was performed during a visit at which an ETT is required, the ETT should be performed a minimum of 30 minutes after Treadmill Familiarization.

Each ETT was conducted according to the modified Gardner protocol at a constant speed of 2.0 mph, with a 2% increase in grade every 2 minutes. The treadmill test began at 2.0 mph, 0% grade, and subsequent increases in grade were made with a programmable treadmill up to a maximum grade of 18%. If the subject could continue walking at this grade without achieving maximal claudication, then the test was continued at 2 mph, 18% grade until the subject achieved MWD. Continuous ECG recording during all ETTs was strongly recommended. Subjects with significant stress-induced ECG abnormalities during the ETT were removed from study participation.

Claudication symptoms were assessed periodically over the course of each ETT, using the Claudication Symptom Rating Scale (1=none; 2=onset; 3=mild; 4=moderate; 5=severe). For each ETT, study staff recorded start time of the test, ICD, MWD and reason for ETT discontinuation. ICD is the onset of claudication of symptoms and was captured in minutes and seconds. MWD occurs when severe claudication symptoms force the cessation of exercise and was captured in minutes and seconds. In case, other signs or symptoms (such as chest pain, dizziness) forced the subject to cease the exercise before the occurrence of leg pain, MWD was still recorded, and the reason for discontinuation was documented.

### Assays for Post-Intervention Cellular Immune Response:

The HLA typing blood test was performed at visit 1a. The HLA antibodies blood test was performed on visits 1a, 2, 4a, 5 and early discontinuation/termination.

HLA-typing and HLA antibodies: Placental ASC lots and subjects were typed by HLA-class I and class II low-resolution typing, using the LABType® SSO (One Lambda, Canoga Park, Calif.). This reverse SSO method is based on a suspension array platform (Luminex®) using microspheres as a solid support to immobilize oligonucleotide probes. The target DNA is amplified by polymerase chain reactions (PCR) and then hybridized with the bead probe array. Alloantibodies in serum were screened at baseline (V1) and after treatment by using a combination of the LABScreen™ for antibody screening and LABScreen Single Antigen test for HLA antibody specification (One Lambda, Canoga Park, Calif.), based on micro beads coated with purified Class I or Class II HLA antigens and pre-optimized reagents for the detection of Class I or Class II HLA antibodies in human sera. LABScreen® products utilize the Lambda Array Beads Multi-Analyte System® (LABMAS), which features the LABScan™ 100 flow analyzer, for data acquisition and analysis. Samples were tested first by using the sensitive LABScreen™ Mixed. This pre-screening test detects the presence or absence of HLA Class I and Class II antibodies. In positive samples, the specificity and percent PRA for HLA Class and Class II was assigned by LABScreen™ PRA Screening test. Specificity was confirmed using the LABScreen™ Single Antigen test. Evaluations were performed by “Fusion software”.

### Drug Randomization

All subjects entered into the study were randomized to receive either placental ASC or placebo, using a blocked randomization procedure. In order to safeguard the double blind nature of the study, the staff members handling the treatment were not allowed to perform the screening and follow-up visits.

### Blinding

Except the unblinded staff members handling the treatment, all investigators and any personnel involved in the subject's assessment, monitoring, analysis, and data management (excluding the designated personnel), were blinded to the subject assignment. In the event of an SAE or pregnancy, when study drug assignment was needed to make treatment decisions for the subject, the investigator was allowed to unblind the subject's drug assignment. In any case, the subject's drug code assignment was not revealed to the sponsor.

The efficacy analysis was performed at 52 weeks, and the primary and secondary efficacy analyses utilized the data collected until week 52.

### Analysis Sets for the First Stage of Efficacy Analysis (52 Weeks)

### Intent-to-Treat Analysis Set (ITT)

The intent-to-treat (ITT) analysis set includes all randomized patients. In this population, treatment was assigned based on the treatment to which patients were randomized, regardless of which treatment they actually received. The ITT analysis set includes efficacy observations that were measured up to week 52. Due to a temporary regulatory concern during the trial, which prevented some patients from receiving the second treatment, this analysis set was used for exploratory purposes only.

### Full Analysis Set (FAS)

The Full Analysis Set (FAS) includes subjects in the ITT analysis set, who received at least one study treatment, and have at least 1 post baseline usable treadmill assessment. The FAS analysis set includes efficacy observations that were measured up to week 65.

Modified Full Analysis Set (mFAS)

The modified full analysis set (mFAS) includes all patients in the ITT analysis set who received at least 1 treatment and had at least 1 post baseline treadmill assessment, excluding those that did not receive the 2nd treatment due to the aforementioned concern. The mFAS analysis set includes efficacy observations that were measured up to week 52.

Full Analysis Set-Subjects that Received 2 Study Treatments (FAS2Rx)

The full analysis set for subjects that received 2 study treatments (FAS2Rx) includes subjects in the FAS analysis set, excluding all subjects that did not receive the 2nd study treatment from any reason (a total of 26 subjects). The FAS2Rx analysis set includes efficacy observations that were measured up to week 65.

### Primary Efficacy End-Point and Analysis

The primary endpoint for this study is log ratio of week 52 MWD to baseline MWD. The principal analysis of the primary endpoint utilizes the Mixed Model for Repeated Measures (MMRM) (SAS® MIXED procedure with REPEATED sub-command) The model includes the following fixed effects: categorical week in trial by treatment interaction, site, and log of baseline MWD measurement. The model uses the unstructured covariance structure and the REML estimation method, and degrees of freedom are adjusted using the Kenward-Roger method. Data from all post-baseline to baseline log ratios visits was used as response in the model, and differences between the treatments groups at week 52 were estimated using contrasts.

Sensitivity Analysis for the Primary Endpoint: The robustness of the principal analysis of the primary endpoint was explored employing the following:

Sensitivity Analysis Using the Last Observed Value (LOV) to baseline log ratio: The LOV-to-baseline log ratio was also calculated and analyzed for the mFAS analysis set. The statistical model was an analysis of covariance (SAS® MIXED procedure), and the model includes treatment group, log MWD baseline value and site effects. The LOV-to-baseline log ratio was used as response variable in the model, and differences between the treatment groups were estimated using contrasts.

Any variable exhibiting a notable imbalance across treatment groups and also known or suspected to be associated to the outcome was assessed for impact on estimated treatment effect using exploratory analyses, where the variable or variables that show an imbalance at baseline and is/are related to outcome was included as covariate(s) in the comparison Week 52/Baseline adjusted MWD models.

### Results

Different doses, schedules, and batches of placental ASC were tested for ability to alleviate intermittent claudication, as below: Group #1: (“low dose”): First and second treatment: 150×10⁶ placental ASC. Group #2: (“high dose”): First and second treatment: 300×10⁶ placental ASC. Group #3: (“placebo”): First and second treatment: Placebo (15mL Vehicle). Group #4: (“single treatment high dose+single treatment placebo”) First treatment: 300×10⁶ placental ASC. Second treatment: Placebo (15mL Vehicle).

Overall, a positive therapeutic effect was observed relative to placebo, particularly with the 300 million dose. Considering the subjects who received the 300 million dose, 2 injections was superior to a single injection (Table 6).

TABLE 6
Log MWD Change - comparison between 1 to 2 injections.

| | Comparison (no. | Ratio | | | Lower 95% | Upper 95% |
|---|---|---|---|---|---|---|
| Week | of injections) | estimate | SE | P-Value | CI Limit | CI Limit |
| 12 | 1-2 | 0.814 | 0.171 | 0.2349 | 0.577 | 1.148 |
| 26 | 1-2 | 0.740 | 0.200 | 0.1399 | 0.495 | 1.107 |
| 39 | 1-2 | 0.740 | 0.202 | 0.1409 | 0.493 | 1.109 |
| 52 | 1-2 | 0.638 | 0.205 | 0.0331 | 0.423 | 0.963 |
| 65 | 1-2 | 0.794 | 0.260 | 0.3793 | 0.472 | 1.337 |

“Ratio estimate” at week 52 is the ratio between the natural Log of MWD at week 52 vs. baseline, which was calculated as follows: the mean change in Log MWD relative to baseline was estimated from the applied statistical model, Mixed Model Repeated Measures (MMRM), for each group as the least squared mean (LSM). The LSM Difference is the difference between the LSM estimates of the indicated groups from the placebo (PBO-PBO) group, and the Ratio Estimate is the exponent of the LSM Difference.

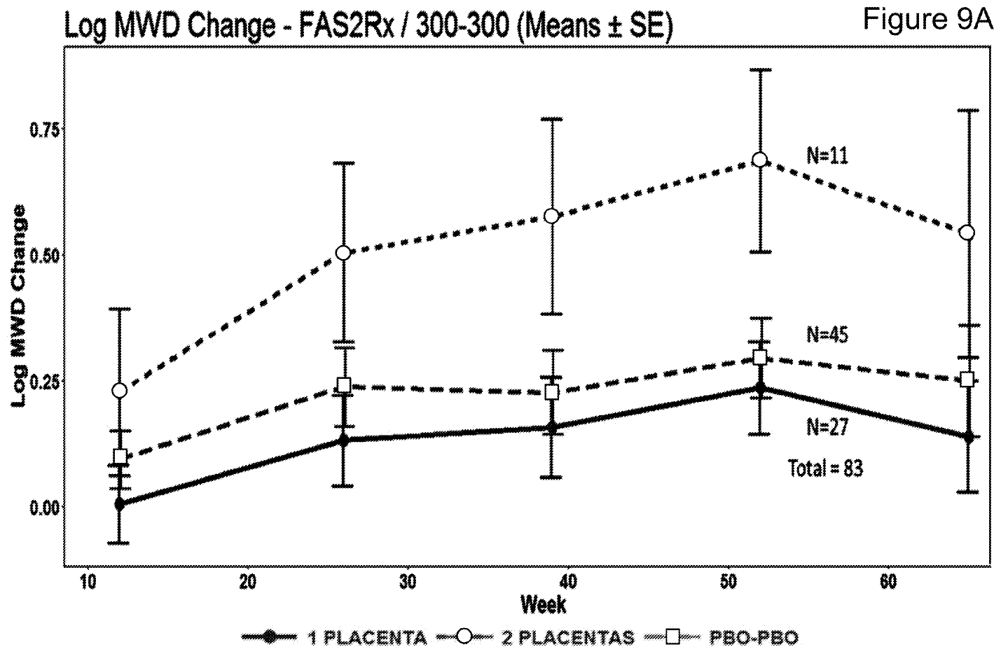

Furthermore, among the subjects receiving 2 injections of 300 million ASC (Group 2), the subjects who received ASC from two different placentas exhibited superior log MWD change than the subjects who received 2 doses of ASC from the same placenta (Table 7 and FIGS. 9A-B).

TABLE 7
Log MWD Change - comparison of subjects treated
with different and same placentas.

| | Treated with | | | | | | |
|---|---|---|---|---|---|---|---|
| | different | | Ratio | | | Lower 95% | Upper 95% |
| Week | placentas | Estimate | estimate | SE | P-value | CI limit | CI limit |
| 12 | No/yes | 0.062 | 0.940 | 0.161 | 0.7035 | 0.679 | 1.302 |
| 26 | No/yes | −0.262 | 0.770 | 0.185 | 0.1651 | 0.529 | 1.119 |
| 39 | No/yes | −0.320 | 0.726 | 0.184 | 0.0894 | 0.500 | 1.053 |
| 52 | No/yes | −0.352 | 0.704 | 0.181 | 0.0590 | 0.488 | 1.014 |
| 65 | No/yes | −0.327 | 0.721 | 0.229 | 0.1601 | 0.454 | 1.144 |

A positive effect relative to placebo was seen when examining both the subjects receiving 2 doses of 300 million ASC from 2 different placentas, and all the subjects that received 2 doses (150 million or 300 million) from 2 different placentas (Table 8).

TABLE 8
Log MWD for subjects with 2 different placenta injections.
Exp(LSM) is the exponent of the LSM (described herein).

| | | | |
|---|---|---|---|
| PBO-PBO | 150M-150M | 300M-300M | ALL ASC |

Baseline

| | | | | |
|---|---|---|---|---|
| n | 44 | 5 | 11 | 16 |
| Mean | 5.65 | 5.80 | 5.34 | 5.48 |
| SD | 0.487 | 0.470 | 0.431 | 0.483 |
| SE | 0.073 | 0.210 | 0.130 | 0.121 |
| Median | 5.73 | 6.04 | 5.37 | 5.40 |
| Min, Max | 4.48, 6.38 | 5.26, 6.31 | 4.47, 6.14 | 4.47, 6.31 |

Week 52 change

| | | | | |
|---|---|---|---|---|
| n | 37 | 4 | 10 | 14 |
| Mean | 0.28 | 0.14 | 0.69 | 0.53 |
| SD | 0.489 | 0.213 | 0.568 | 0.546 |
| SE | 0.080 | 0.106 | 0.179 | 0.146 |
| Median | 0.29 | 0.18 | 0.57 | 0.39 |
| Min, Max | −0.61, 1.63 | −0.11, 0.32 | 0.05, 1.93 | −0.11, 1.93 |
| LS Mean | 0.254 | 0.075 | 0.605 | 0.430 |
| SE | 0.096 | 0.230 | 0.170 | 0.148 |
| Exp(LS Mean) | 1.289 | 1.078 | 1.831 | 1.538 |
| Exp(95% CI) | 1.064, 1.563 | 0.681, 1.705 | 1.303, 2.571 | 1.145, 2.064 |
| p-value(LS Mean) | 0.0103 | 0.7459 | 0.0007 | 0.0048 |
| LS Mean | | −0.179 | 0.350 | 0.175 |
| Difference | | | | |
| 95% CI | | −0.639, 0.281 | 0.012, 0.689 | −0.121, 0.471 |
| Ratio Estimate | | 0.836 | 1.420 | 1.191 |
| (Exp(LSM Dif)) | | | | |
| 95% CI of Ratio | | 0.528, 1.324 | 1.012, 1.992 | 0.886, 1.601 |
| Estimate | | | | |
| p-value | | 0.4379 | 0.0429 | 0.2416 |

Furthermore, reduced revascularization rates up to week 65 were observed in subjects treated with 2 different placentas (Table 9), more so than with the overall population of treated subjects (Table 10).

TABLE 9
Revascularization rates up to week 65 in
subjects receiving 2 different placentas.

| At least 1 | | | |
|---|---|---|---|
| revascularization | | | |
| event (%) | PBO-PBO | 150M-150M | 300M-300M |
| Week 52 - n | 44 | 5 | 11 |
| No | 40 (90.9) | 5 (100) | 11 (100) |
| Yes | 4 (9.1) | 0 | 0 |
| Week 65 - n | 44 | 5 | 11 |
| No | 38 (86.4) | 5 (100) | 11 (100) |
| Yes | 6 (13.6) | 0 | 0 |

TABLE 10
First revascularization up to week 65 in treated subjects overall.

| At least 1 | | | | |
|---|---|---|---|---|
| revascularization | | | | |
| event (%) | PBO-PBO | 300M-PBO | 150M-150M | 300M-300M |
| Week 52 - n | 50 | 37 | 37 | 48 |
| No | 46 (92.0) | 31 (83.8) | 30 (81.1) | 45 (93.8) |
| Yes | 4 (8.0) | 6 (16.2) | 7 (18.9) | 3 (6.3) |
| Week 65 - n | 50 | 37 | 37 | 48 |
| No | 44 (88.0) | 31 (83.8) | 30 (81.1) | 45 (93.8) |
| Yes | 6 (12.0) | 6 (16.2) | 7 (18.9) | 3 (6.3) |

Moreover, a significant reduction in Hemoglobin A1C (HbA1C) was observed in subjects that received either 1 or 2 doses of 300 million ASC. The reduction was even sharper in subjects who received ASC from two different placentas (Table 11). Subjects in the different groups had similar baseline HbA1C values (Table 12).

TABLE 11
Week 65 ANCOVA of Change from Baseline in HbA1C
(mmol/mol). Difference of Adjusted Means.

| | | | | Lower 95% | Upper 95% |
|---|---|---|---|---|---|
| Comparison | Estimate | SE | P-Value | CI Limit | CI Limit |
| 300M-PBO - PBO-PBO | −4.414 | 1.978 | 0.0273 | −8.326 | −0.503 |
| 150M-150M - PBO-PBO | 0.740 | 0.200 | 0.1399 | 0.495 | 1.107 |
| 300M-300M - PBO-PBO | −2.147 | 1.941 | 0.2706 | −5.986 | 1.691 |
| 300M-300M different | −7.770 | 3.087 | 0.0155 | −13.988 | −1.553 |
| placentas - PBO-PBO | | | | | |

TABLE 12
Baseline HbA1C values in the study groups.

| Group | N | Mean | SD |
|---|---|---|---|
| PBO-PBO | 43 | 46.13 | 11.75 |
| 300M-PBO | 30 | 43.93 | 9.90 |
| 150M-150M | 32 | 43.38 | 9.84 |
| 300M-300M | 40 | 44.34 | 8.71 |

300M-300M subgroups (only subjects with HbA1c data at wk 65)

| | | | |
|---|---|---|---|
| 300M-300M different placentas | 11 | 48.1 | 7.7 |
| 300M-300M same placentas | 23 | 43.4 | 11.1 |

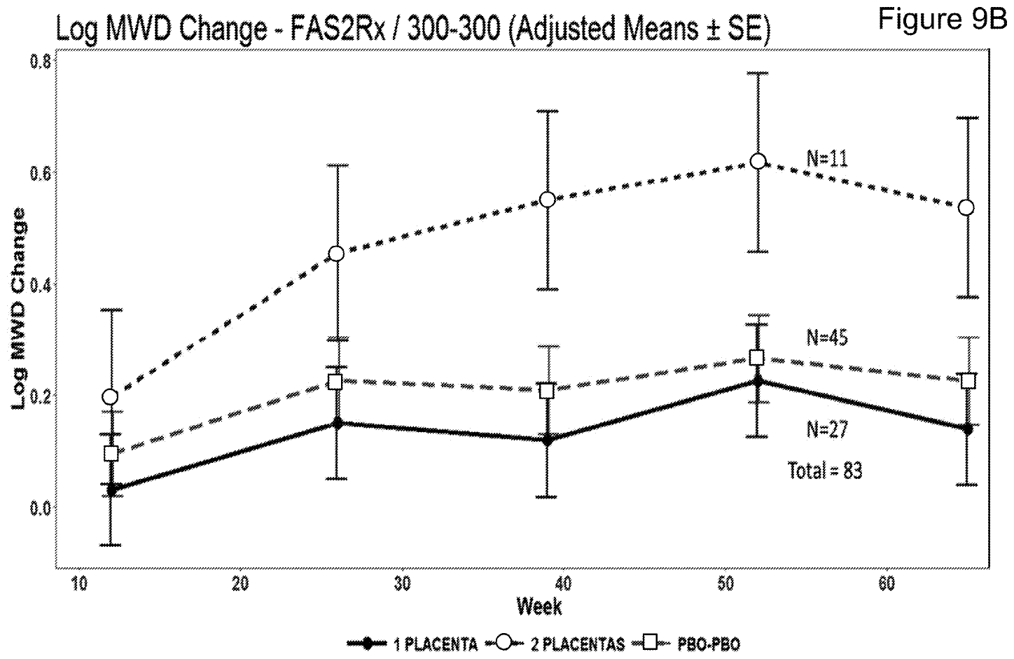

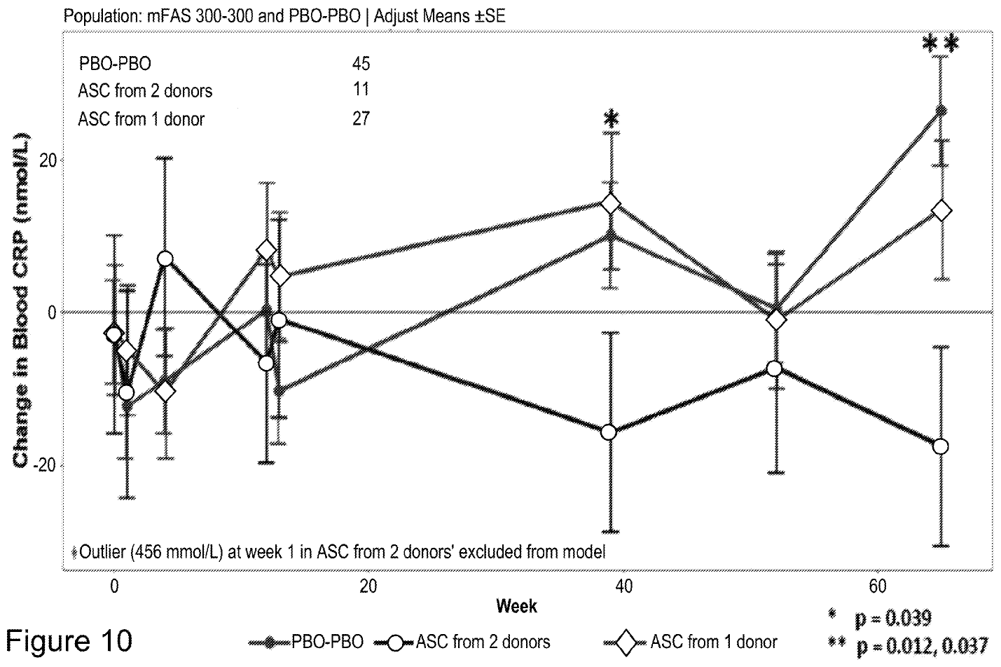

Additionally, subjects who received ASC from two different placentas exhibited a reduction from baseline CRP levels, which was not seen in the PBO group (FIG. 10 and Table 13).

TABLE 13
Week 65 Descriptive Statistics of Change
from Baseline in Blood CRP (nmol/L).

| | | Treatment | | |
|---|---|---|---|---|
| | | 300M-300M from | 300M-300M from | |
| | | same donor | different donors | PBO-PBO |
| Baseline | N | 24 | 11 | 40 |
| | Mean | 28.4 | 45.5 | 32.9 |
| | SD | 27.5 | 47.4 | 38.8 |
| | Min | 0.1 | 0.1 | 0.1 |
| | Median | 17.7 | 28.6 | 22.4 |
| | Max | 111.4 | 151 | 189.3 |
| Change from | N | 24 | 11 | 40 |
| Baseline | Mean | 13.8 | −7.5 | 29 |
| | SD | 67.9 | 56.8 | 95 |
| | Min | −41.9 | −125.9 | −58.1 |
| | Median | −0.2 | −1.4 | 1.4 |
| | Max | 297.6 | 83 | 521.1 |

Additionally, the safety profile of the placental ASC was excellent. Most categories of adverse events were either unaffected or reduced (Table 14).

TABLE 14
Adverse events in the study groups.

| | PBO-PBO | 300-PBO | 150-150 | 300-300 |
|---|---|---|---|---|
| | (n = 51) | (n = 36) | (n = 37) | (n = 48) |
| Death | 0% | 0% | 0% | 2.1% |
| Major amputations | 3.9% | 0% | 0% | 0% |
| Malignancies | 7.8% | 5.6% | 10.8% | 2.1% |
| Infections | 33.3% | 22.2% | 32.4% | 33.3% |
| Injection site pain | 39.2% | 30.6% | 40.5% | 47.9% |
| hematoma | 9.8% | 2.8% | 5.4% | 6.3% |
| → leading to discontinuation | 0% | 2.8% | 2.7% | 4.2% |
| Peripheral vascular disorders | 29.4% | 27.8% | 27.0% | 22.9% |
| Cardiac disorders | 9.8% | 11.1% | 8.1% | 6.3% |
| Neurologic disorders | 27.5% | 13.9% | 18.9% | 20.8% |
| Blood and lymphatic disorders | 9.8% | 8.3% | 2.7% | 2.1% |
| Renal disorders | 9.8% | 8.3% | 5.4% | 6.3% |
| Ophthalmologic disorders | 11.8% | 5.6% | 5.4% | 4.2% |
| Respiratory tract disorders | 17.6% | 2.8% | 10.8% | 8.3% |
| Abnormal lab findings | 13.7% | 8.3% | 8.1% | 6.3% |
| Gastrointerstinal disorders | 23.5% | 25.0% | 18.9% | 20.8% |
| Musculosceletal disorders | 39.2% | 41.7% | 35.1% | 31.1% |
| Psychiatric disorders | 7.8% | 5.6% | 0% | 4.2% |

### Example 6: Reduced Anti-HLA Antibodies in Patients Receiving ASC From 2 Different Placentas

Methods

Where sufficient blood samples were available, subjects were analyzed for anti-HLA antibodies. The presence and specificity of HLA antibodies have been determined by LABScreen® Mixed (One Lambda, Canoga Park, Calif., USA) and LABScreen® Single Antigen (One Lambda), respectively. The percentage PRA score represents the proportion of the population to which the subject reacts via pre-existing antibodies against human HLA class I.

Results

7% of the subject from the study described in the previous Example were pre-sensitized to HLA. The remaining patients that received 2 doses of ASC were examined for the presence of anti-HLA antibodies at visit 5, one week after the second treatment. The patients receiving different ASC has a lower incidence of both anti-HLA antibodies and a lower average panel PRA score (Table 15). Further, all 4 of patients in the mismatch group who developed anti-HLA antibodies had received ASC from different placentas that share 2 common alleles, one in A and one in B. Patients that received ASC from different placentas that did not share common HLA-A and HLA-B alleles, for example 04 and 27, did not develop anti-HLA antibodies.

TABLE 15
Percentage of subjects with anti-HLA antibodies and
average PRA score at visit 5 in patients receiving
2 doses of ASC from the same or different placentas.

| | Match | Mismatch |
|---|---|---|
| Anti-HLA antibodies | 63% (10/16) | 50% (4/8) |
| Average PRA score | 42.75% | 30.75% |

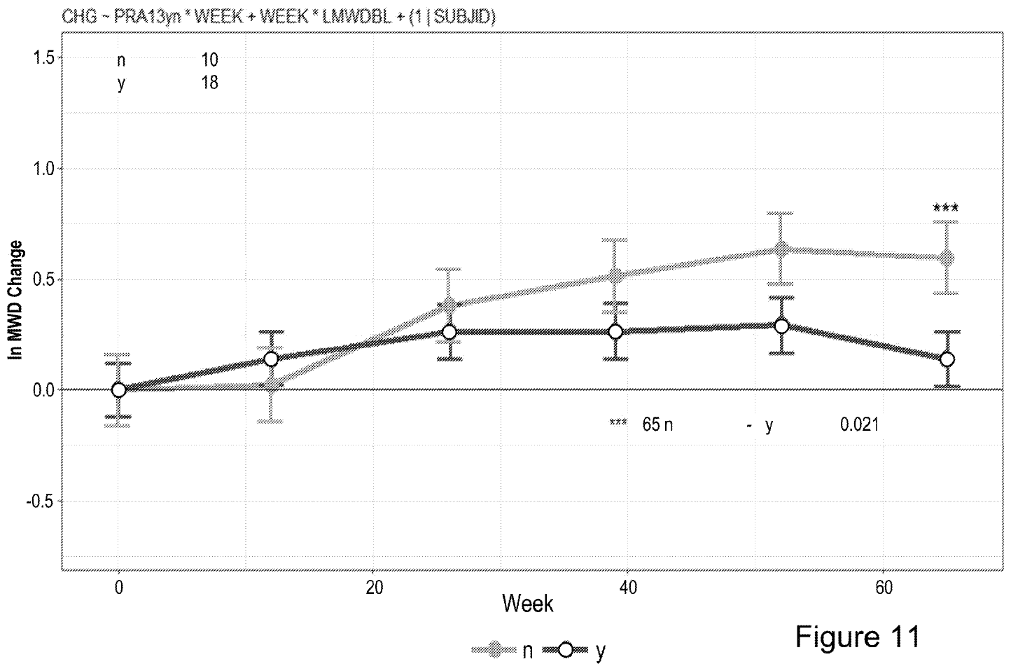

Lack of antibodies at visit 5 also correlated with improved MWD response (FIG. 11), further corroborating the advantages of administration of non-matched ASC.

### Example 7: Osteocyte and Adipocyte Differentiation Assays

ASC were prepared as described in Example 1. BM adherent cells were obtained as described in WO 2016/098061 to Esther Lukasiewicz Hagai and Rachel Ofir, which is incorporated herein by reference in its entirety. Osteogenesis and adipogenesis assays were performed as described in WO 2016/098061, which is incorporated herein by reference.

Osteocyte induction. Incubation of BM-derived adherent cells in osteogenic induction medium resulted in differentiation of over 50% of the BM cells, as demonstrated by positive alizarin red staining while none of the placental ASC exhibited signs of osteogenic differentiation. Next, a modified osteogenic medium comprising Vitamin D and higher concentrations of dexamethasone was used. Over 50% of the BM cells underwent differentiation into osteocytes, while none of the placental ASC exhibited signs of osteogenic differentiation.

Adipocyte induction. Adipocyte differentiation of placenta ASC or BM adherent cells in adipocyte induction medium resulted in differentiation of over 50% of the BM-derived cells, as demonstrated by positive oil red staining and by typical morphological changes (accumulation of oil droplets in the cytoplasm). In contrast, none of the placental ASC differentiated into adipocytes. Next, a modified medium containing a higher indomethacin concentration was used. Over 50% of the BM-derived cells underwent differentiation into adipocytes. In contrast, none of the placental ASC exhibited morphological changes typical of adipocytes.

### Example 8: Further Osteocyte and Adipocyte Differentiation Assays

ASC were prepared as described in Examples 2-3. Adipogenesis and osteogenesis were assessed using the STEMPRO® Adipogenesis Differentiation Kit (GIBCO, Cat#A1007001) and the STEMPRO® Osteogenesis Differentiation Kit (GIBCO, Cat#A1007201), respectively.

Results

Adipogenesis and osteogenesis of placental ASC grown in SRM or in full DMEM, and BM-MSC, were tested. BM-MSC treated with adipogenesis differentiation medium stained positively with Oil Red O. By contrast, ⅔ of the ASC/SRM batches exhibited negligible staining, and the other ASC/SRM batch, as well as the full DMEM-grown cells, exhibit no staining at all, showing lack of significant adipogenic potential. In osteogenesis assays, BM-MSCs treated with differentiation medium stained positively with Alizarin Red S, while, none of the placental batches grown in SRM or full DMEM exhibited staining, showing lack of significant osteogenic potential.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### Additional References may be Cited in Text

Alelign T et al, Kidney Transplantation: The Challenge of Human Leukocyte Antigen and Its Therapeutic Strategies. J Immunol Res. 2018 Mar. 5; 2018:5986740. doi: 10.1155/2018/5986740. eCollection 2018.
Chase L G et al, Development and Characterization of a Clinically Compliant Xeno-Free Culture Medium in Good Manufacturing Practice for Human Multipotent Mesenchymal Stem Cells. Stem Cells Transl Med. 2012 October; 1(10): 750-758.
Chin et al, “Clinical usefulness of a novel C1q assay to detect immunoglobulin G antibodies capable of fixing complement in sensitized pediatric heart transplant patients,” The Journal of Heart and Lung Transplantation, vol. 30, no. 2, pp. 158-163, 2011.
Dominici et al. Minimal criteria for defining multipotent mesenchymal stromal cells. The International Society for Cellular Therapy position statement. Cytotherapy. 2006; 8(4):315-7.
Gardner A W, Skinner J S, Cantwell B W, Smith L K. Progressive vs single-stage treadmill tests for evaluation of claudication. Med Sci Sports Exerc. 1991; 23:402-8.
Hiatt W R, Nawaz D, Regensteiner J G, Hossack K F. The evaluation of exercise performance in patients with peripheral vascular disease. J Cardiopulmonary Rehabil. 1988; 12:525-32
Imamura. Physiological Functions and Underlying Mechanisms of Fibroblast Growth Factor (FGF) Family Members: Recent Findings and Implications for Their Pharmacological Application. Biological and Pharmaceutical Bulletin, Vol. 37(7):1081-1089 (2014).
Kannagi R et al, Stage-specific embryonic antigens (SSEA-3 and -4) are epitopes of a unique globo-series ganglioside isolated from human teratocarcinoma cells. EMBO J. 1983; 2(12):2355-61.
Kinzebach S and Bieback K. Expansion of Mesenchymal Stem/Stromal cells under xenogenic-free culture conditions. Adv Biochem Eng Biotechnol. 2013; 129:33-57.
Miedel M C et al, Structural analysis of recombinant soluble human interleukin-2 receptor. Primary structure, assignment of disulfide bonds and core IL-2 binding structure. Biochem. Biophys. Res. Commun. 154 (1), 372-379 (1988)
Nicolai S P et al. Reliability of treadmill testing in peripheral arterial disease: a meta-regression analysis. J Vasc Surg. 2009; 50:322-9.
Nomura T et al, Clin Cancer Res. 2006; 12(24):7294-305. Beta2-microglobulin promotes the growth of human renal cell carcinoma through the activation of the protein kinase A, cyclic AMP-responsive element-binding protein, and vascular endothelial growth factor axis.
Robinson J, Halliwell J A, Hayhurst J H, Flicek P, Parham P, Marsh S G E: The IPD and IMGT/HLA database: allele variant databases. Nucleic Acids Research. 2015 43:D423-431.
Sidney J, Peters B, Frahm N, Brander C, Sette A. HLA class I supertypes: a revised and updated classification. BMC Immunol. 2008 Jan. 22; 9:1. doi: 10.1186/1471-2172-9-1.
Silva et al., “Evaluation of HLA matchmaker compatibility as predictor of graft survival and presence of anti-HLA antibodies,” Transplantation Proceedings, vol. 42, no. 1, pp. 266-269, 2010.
Valenzuela and Reed, “Antibodies in transplantation: the effects of HLA and non-HLA antibody binding and mechanisms of injury,” in Transplantation Immunology. Methods in Molecular Biology (Methods and Protocols), vol. 1034, pp. 41-70, Humana Press, Totowa, N.J., USA, 2013.
Winkler T et al , Immunomodulatory placental-expanded, mesenchymal stromal cells improve muscle function following hip arthroplasty. J Cachexia Sarcopenia Muscle. 2018 October; 9(5):880-897. doi: 10.1002/jcsm.12316.

What is claimed is:

1. A therapeutic method, comprising:
a. administering to a subject a first pharmaceutical composition, comprising allogeneic adherent stromal cells (ASC) from a first donor; and
b. administering to said subject, at least 7 days after step a), a second pharmaceutical composition comprising allogeneic ASC from a second donor, wherein said second donor differs from said first donor in at least one allele group of human leukocyte antigen (HLA)-A or human leukocyte antigen (HLA)-B.

2. The therapeutic method of claim 1, wherein said second donor differs from said first donor in at least one allele group of HLA-A.

3. The therapeutic method of claim 1, wherein said second donor differs from said first donor in at least one allele group of HLA-B.

4. The therapeutic method of claim 1, wherein said second donor differs from said first donor in both allele groups of HLA-A or HLA-B.

5. The therapeutic method of claim 1, wherein said second donor differs from said first donor in at least one allele supertype of HLA-A or HLA-B.

6. The therapeutic method of claim 1, wherein said second donor differs from said first donor in both allele supertypes of HLA-A.

7. The therapeutic method of claim 1, wherein step b) is performed between 2-52 weeks after step a).

8. (canceled)

9. The therapeutic method of claim 1, wherein step b) is performed between 6-20 weeks after step a).

10. The therapeutic method of claim 1, further comprising administering to said subject, at least 7 days after step b), a third pharmaceutical composition comprising allogeneic ASC of a third donor, wherein said third donor differs from both said first donor and said second donor in at least one allele group of HLA-A or HLA-B.

11-21. (canceled)

22. The method of claim 1, wherein said ASC originate from placenta tissue.

23. The method of claim 22, wherein said ASC express a marker selected from the group consisting of CD73, CD90, CD29 and CD105.

24. The method of claim 22, wherein said ASC do not express a marker selected from the group consisting of CD3, CD4, CD11b, CD14, CD19, and CD34.

25. The method of claim 22, wherein said ASC do not express a marker selected from the group consisting of CD3, CD4, CD34, CD39, and CD106.

26. The method of claim 25, wherein less than 50% of said ASC express CD200.

27. The method of claim 25, wherein more than 50% of said ASC express CD200.

28. The method of claim 25, wherein more than 50% of said ASC express CD141 or SSEA4.

29. The method of claim 25, wherein more than 50% of said ASC express HLA-A2.

30. The method of claim 1, wherein said ASC originate from adipose tissue or bone marrow.

31. The method of claim 1, wherein the cells are administered intramuscularly.

32. The method of claim 1, wherein the cells are administered intravenously, subcutaneously, or intraperitoneally.


SEQUENCE LISTING (10 sequences)

SEQ ID NO: 1 - protein, 371 aa, Homo sapiens [sequence omitted]

SEQ ID NO: 2 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 3 - protein, 365 aa, Homo sapiens [sequence omitted]

SEQ ID NO: 4 - protein, 365 aa, Homo sapiens [sequence omitted]

SEQ ID NO: 5 - protein, 365 aa, Homo sapiens [sequence omitted]

SEQ ID NO: 6 - protein, 362 aa, Homo sapiens
MLVMAPRTVLLLLSAALALTETWAGSHSMRYFYTAVSRPGRGEPRFISVGYVDDTQFVRFDSDAASPREEPRAPWIEQEGPEYWDRNTQICKTNTQTDRESLRNLRGYYNQSEAGSHTLQWMYGCDVGPDGRLLRGYNQFAYDGKDYIALNEDLSSWTAADTAAQITQRKWEAAREAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAASSDSAQGSDVSLTA

SEQ ID NO: 7 - protein, 362 aa, Homo sapiens
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 8 - protein, 362 aa, Homo sapiens
MRVTAPRTVLLLLSGALALTETWAGSHSMRYFYTAMSRPGRGEPRFIAVGYVDDTQFVRFDSDAASPRMAPRAPWIEQEGPEYWDRETQISKTNTQTYRESLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTIPIVGIVAGLAVLAVVVIGAVVATVMCRRKSSGGKGGSYSQAASSDSAQGSDVSLTA

SEQ ID NO: 9 - protein, 365 aa, Homo sapiens
MRVTAPRTVLLLLWGAVALTETWAGSHSMRYFYTAMSRPGRGEPRFIAVGYVDDTQFVRFDSDAASPRTEPRAPWIEQEGPEYWDRETQISKTNTQTYRENLRIALRYYNQSEAGSHTWQTMYGCDVGPDGRLLRGHNQYAYDGKDYIALNEDLSSWTAADTAAQITQRKWEAAREAEQLRAYLEGLCVEWLRRHLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSSQSTIPIVGIVAGLAVLAVVVIGAVVATVMCRRKSSGGKGGSYSQAACGKPATVPRALMCLSQL

SEQ ID NO: 10 - protein, 362 aa, Homo sapiens
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQLRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA